(19) United States
(12) Patent Application Publication
(10) Pub. No.: US 2022/0354857 A1
(43) Pub. Date: Nov. 10, 2022

(54) ALDEHYDE CONJUGATES AND USES THEREOF

(71) Applicant: Aldeyra Therapeutics, Inc., Lexington, MA (US)

(72) Inventors: Todd Brady, Carlisle, MA (US); Scott Young, E. Falmouth, MA (US); William A. Kinney, Newtown, PA (US)

(21) Appl. No.: 17/229,797

(22) Filed: Apr. 13, 2021

Related U.S. Application Data
(63) Continuation of application No. 16/592,572, filed on Oct. 3, 2019, now abandoned.
Application No. 16/592,572 is a continuation of application No. 15/754,163, filed on Feb. 21, 2018, now abandoned.
(60) Provisional application No. 62/208,278, filed on Aug. 21, 2015.
(60) Provisional application No. 62/315,455, filed on Mar. 30, 2016.
(60) Provisional application No. 62/347,464, filed on Jun. 8, 2016.

(51) Int. Cl. A61K 31/5365; A61Q 19/08; A61K 8/49; A61K 31/4365; A61K 8/69; A61K 31/47; C07D 498/04; C07D 498/10; A61P 27/02; A61P 17/00

(52) U.S. Cl. CPC A61K 31/5365; A61Q 19/08; A61K 8/4926; A61K 31/4365; A61K 8/69; A61K 8/49; A61K 31/42; A61K 31/47; C07D 498/04; C07D 498/10; A61P 27/02; A61P 17/00; A61K 8/494

(57) ABSTRACT

The present invention provides compounds and methods of use thereof for the treatment, prevention, and/or reduction of a risk of a disease, disorder, or condition in which aldehyde toxicity is implicated in the pathogenesis, including ocular disorders, skin disorders, conditions associated with injurious effects from blister agents, and autoimmune, inflammatory, neurological and cardiovascular diseases by the use of a primary amine to scavenge toxic aldehydes, such as MDA and HNE.

### BACKGROUND OF THE INVENTION

Metabolic and inflammatory processes in cells generate toxic aldehydes, such as malondialdehyde (MDA) and 4-hydroxyl-2-nonenal (HNE or 4HNE). These aldehydes are highly reactive to proteins, carbohydrates, lipids and DNA, leading to chemically modified biological molecules, activation of inflammatory mediators such as NF-κB, and damage in diverse organs. For example, retinaldehyde can react with phosphatidylethanolamine (PE) to form a highly toxic compound called A2E, which is a component of lipofuscin believed to be involved in the development and progression of Age Related Macular Degeneration (AMD). Many bodily defense mechanisms function to remove or lower the levels of toxic aldehydes. Novel small molecule therapeutics can be used to scavenge “escaped” retinaldehyde in the retina, thus reducing A2E formation and lessening the risk of AMD (see, WO 2006/12794).

Aldehydes are implicated in diverse pathological conditions such as dry eye, cataracts, keratoconus, Fuch's endothelial dystrophy in the cornea, uveitis, allergic conjunctivitis, ocular cicatricial pemphigoid, conditions associated with photorefractive keratectomy (PRK) healing or other corneal healing, conditions associated with tear lipid degradation or lacrimal gland dysfunction, inflammatory ocular conditions such as ocular rosacea (with or without meibomian gland dysfunction), and non-ocular disorders or conditions such as skin cancer, psoriasis, contact dermatitis, atopic dermatitis, acne vulgaris, Sjogren-Larsson Syndrome, ischemic-reperfusion injury, inflammation, diabetes, neurodegeneration (e.g., Parkinson's disease), scleroderma, amyotrophic lateral sclerosis, autoimmune disorders (e.g., lupus), cardiovascular disorders (e.g., atherosclerosis), and conditions associated with the injurious effects of blister agents (Negre-Salvagre et al., 2008, Br J Pharmacol. 153(1):6-20; Nakamura et al., 2007, Invest Ophthalmol Vis Sci 48: 1552; Batista et al., 2012, Molecular Vision 18:194; Kenney et al., 2003; Baz et al., 2004, Int J Dermatol 43:494; Augustin et al., 1994, Graefe's Clin Exp Ophthalmol. 233:694). Reducing or eliminating aldehydes should thus ameliorate the symptoms and slow the progression of these pathological conditions.

MDA, HNE and other toxic aldehydes are generated by a myriad of metabolic mechanisms involving: fatty alcohols, sphingolipids, glycolipids, phytol, fatty acids, arachidonic acid metabolism (Rizzo et al., 2007, Mol Genet Metab. 90(1):1-9), polyamine metabolism (Wood et al. (2006)), lipid peroxidation, oxidative metabolism (Buddi et al., 2002, J Histochem Cytochem. 50(3):341-51; Zhou et al., 2005, Exp Eye Res. 80(4):567-80; Zhou et al., 2005, J Biol Chem. 280(27):25377-82), and glucose metabolism (Pozzi et al., 2009, J Am Soc Nephrol. 20(10):2119-25). Aldehydes can crosslink with primary amino groups and other chemical moieties on proteins, phospholipids, carbohydrates, and DNA, leading in many cases to toxic consequences, such as mutagenesis and carcinogenesis (Marnett, 2002, Toxicology. 181-182:219-22.). MDA is associated with diseased corneas, keratoconus, bullous and other keratopathy, and Fuch's endothelial dystrophy corneas (Buddi et al., supra). Also, skin disorders, e.g., Sjogren-Larsson Syndrome, are likely connected with the accumulation of fatty aldehydes such as octadecanal and hexadecanal (Rizzo et al., 2010, Arch Dermatol Res. 302(6):443-51). Further, increased lipid peroxidation and resultant aldehyde generation are associated with the toxic effects of blister agents (Sciuto et al., 2004, Inhal Toxicol. 16(8):565-80; and Pal et al., 2009, Free Radic Biol Med. 47(11):1640-51).

There has been no suggestion in the art for treating the various conditions associated with toxic aldehydes by the administration of small molecule therapeutics acting as a scavenger for aldehydes, such as MDA and/or HNE. Thus, there is a need for treating, preventing, and/or reducing a risk of a disease or disorder in which aldehyde toxicity is implicated in the pathogenesis. The present invention addresses such a need.

Accordingly, there remains a need for treating, preventing, and/or reducing a risk of a disease or disorder in which aldehyde toxicity is implicated in the pathogenesis.

### SUMMARY OF THE INVENTION

It has now been found that compounds of the present invention, and compositions thereof, are useful for treating, preventing, and/or reducing a risk of a disease, disorder, or condition in which aldehyde toxicity is implicated in the pathogenesis. Such compounds have general formula I and are generated through the reaction of an amino-carbinol with a biologically relevant aldehyde:

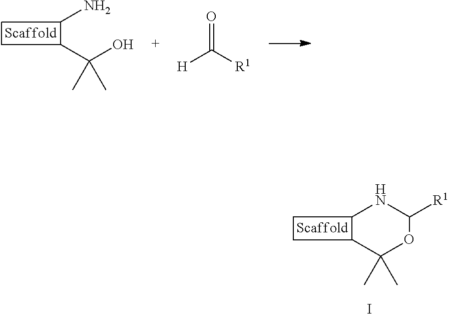

or a pharmaceutically acceptable salt thereof, wherein each of R¹ and Scaffold is as defined herein and described in embodiments.

### BRIEF DESCRIPTION OF THE FIGURES

FIG. 1 shows profiles of NS2 levels and time courses of NS2-SSA adduct formation in serum, brain and liver of wild type mice after administration of a single dose of NS2.

FIG. 2 shows levels of NS2-SSA adducts in tissues from wild type mice and SSADH-deficient mice.

FIG. 3 shows brain, liver, and kidney levels of NS2-SSA adduct after NS2 administration as a single dose to SSADH knock-out mice.

FIG. 4 shows levels of GHB, SSA and D-2-HG in tissues from wild type and SSADH null mice treated with vehicle or NS2.

FIG. 5 shows the GHB/SSA and D-2-HG/SSA levels of SSADH null mice (22-23 days old) who received one dose of 10 mg/kg NS2 or vehicle (IP) compared with those of wild type mice. Brain, liver and kidney were harvested 8 hours following treatment (statistical analysis: student's t test (**p<0.01)).

FIG. 6 shows levels of NS2-SSA adduct in tissues from wild type and SSADH null mice treated with vehicle or NS2.

FIG. 7 shows photomicrographs of cardiac fibroblasts stained for vimentin (red) and a-SMA (green) with DAPI (blue) to mark the nuclei: (A) Cells at initial plating showing small rounded cells with no α-SMA; (B) Unstimulated cells showing a marked change in morphology and an increase in a-SMA; and (C) H₂O₂ stimulated cells showing strong upregulation of α-SMA and dramatic changes in cell shape.

FIG. 8 shows photomicrophaphs of unstimulated cardiac fibroblasts stained for α-SMA (green), vimentin (red) and DAPI (blue) with the following treatments: (A) and (E) no NS2; (B) and (F) 10 μM NS2; (C) and (G) 100 μM NS2; (D) and (H) 1 mM NS2. Panels E-H are higher magnification of a subset of cells to show the change in morphology with NS2 treatment.

FIG. 9 shows photomicrographs of H₂O₂ stimulated cardiac fibroblasts stained for α-SMA (green), vimentin (red) and DAPI (blue) with the following treatments: (A) and (E) no NS2; (B) and (F) 10 μM NS2; (C) and (G) 100 μM NS2; (D) and (H) 1 mM NS2. Panels E-H are higher magnification of a subset of cells to show the change in morphology with NS2 treatment.

FIG. 10 shows: (A) Western Blots of α-SMA levels in cardiac fibroblasts, and (B) Effect of NS2 treatment on α-SMA in unstimulated and H₂O₂ stimulated cells, with NS2 treatment showing significant decrease in α-SMA levels at all doses in unstimulated cells and at the higher doses in H₂O₂ stimulated cells.

FIG. 11 shows photomicrographs of cells stained to DAP (blue) and NFκB (red) and show NFκB translocation to the nucleus of unstimulated cardiac fibroblasts: (A) Examination of separate channels shows NS2 treatment limits NFκB translocation; and (B) Statistical analysis of % cells with nuclear NFκB. NS2 at 1 mM did not have enough cells for analysis and thus is not presented.

FIG. 12 shows: (A) Western Blot of NFκB in both unstimulated and stimulated cardiac fibroblasts; and (B) Statistical analysis showing that NS2 significantly decreases NFκB levels at all doses in unstimulated cells and at the higher doses in H₂O₂ stimulated cells.

FIG. 13 shows: (A) Western Blot of IL-1β levels in unstimulated and H₂O₂ stimulated cardiac fibroblasts; and (B) Density of IL-1β levels, showing that NS2 significantly decreases IL-1β levels at all doses in both unstimulated and H₂O₂ stimulated fibroblasts.

FIG. 14 shows Western Blot of members of MAPK family of proteins: (A) ERK and phosphor-ERK; (B) JNK and phosphor-JNK; and (C) p38 and phosphor-p38. No clear changes in phosphorylation were seen.

FIG. 15 shows rates of formation of aldehyde adducts over a 23 h time period for NS2 and exemplary compounds of the present invention.

FIG. 16 shows consumption of 4HNE over time (23-hour formation period) for NS2 and exemplary compounds of the present invention.

FIG. 17 shows rates of formation of aldehyde adducts over a 1 week time period for NS2 and exemplary compounds of the present invention to measure whether compounds reached equilibrium. During this time period 3 of the 5 samples reached equilibrium.

FIG. 18 shows consumption of 4HNE over a 1 week time period for NS2 and exemplary compounds of the present invention to measure whether compounds reached equilibrium during this time period. The samples appeared to reach equilibrium, with the ongoing decrease in 4HNE amounts possibly due to another degradative pathway.

### DETAILED DESCRIPTION OF THE INVENTION

### 1. General Description of Certain Aspects of the Invention

As described above, biologically relevant aldehydes are associated with a variety of disorders. In addition, certain compounds, described in detail herein, having an amino carbinol moiety are useful as “aldehyde traps.” Such amino-carbinol containing compounds can react with the aldehyde moiety in vitro or in vivo thereby effectively “trapping” the biologically relevant aldehyde and rendering it unreactive. Thus, in some embodiments, the present invention provides a method comprising the steps of:
(a) providing a compound of formula A:

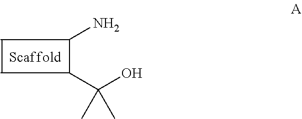

or a pharmaceutically acceptable salt thereof, wherein:
Scaffold is a moiety to which the amino group and the carbinol group are attached such that the resulting amino-carbinol moiety is capable of trapping an aldehyde moiety; and
(b) contacting the compound of formula A with a biologically relevant aldehyde to form a conjugate of formula I:

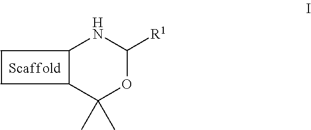

wherein:
R¹ is the side-chain of the biologically relevant aldehyde.

### 2. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.”

The term “aryl” used alone or as part of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen, —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurances of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurances of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{*}₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurances of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, besylate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

### 3. Description of Exemplary Compounds

As described above, biologically relevant aldehydes are associated with a variety of disorders. In addition, certain compounds, such as those of formulae II, III, IV-A, and IV-B, described in detail below, having an amino-carbinol moiety are useful as “aldehyde traps.” Such amino-carbinol containing compounds can react with the aldehyde moiety in vitro or in vivo thereby effectively “trapping” the biologically relevant aldehyde and rendering it unreactive. Thus, in some embodiments, the present invention provides a method comprising the steps of:
(a) providing a compound of formula A:

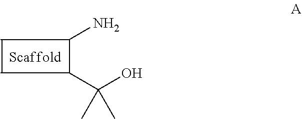

or a pharmaceutically acceptable salt thereof, wherein:
Scaffold is a moiety to which the amino group and the carbinol group are attached such that the resulting amino-carbinol moiety is capable of trapping an aldehyde moiety; and
(b) contacting the compound of formula A with a biologically relevant aldehyde to form a conjugate of formula I:

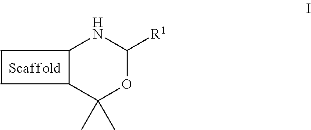

wherein:
R¹ is the side-chain of a biologically relevant aldehyde.

In some embodiments, Scaffold provides a compound of formula A, selected from any of those recited in published international patent application WO 2014/116836 (PCT/US2014/012762), herein referred to as the '836 publication, the entirety of which is incorporated herein by reference.

In some embodiments, Scaffold provides a compound of formula A, selected from any of those recited in U.S. Pat. No. 7,973,025, the entirety of which is incorporated herein by reference.

In some embodiments, Scaffold provides a compound of formula A, selected from those of formula II:

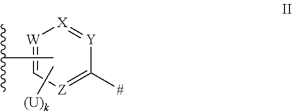

or a pharmaceutically relevant salt, wherein:
[symbol] is the point of attachment to the amine group;
# is the point of attachment to the carbinol group;
each W, X, Y, or Z is independently selected from N, O, S, CU, or CH;
k is 0, 1, 2, 3, or 4;
each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
two occurances of U on adjacent carbon atoms can form an optionally substituted fused ring, selected from a fused phenyl ring; a fused 5-6 membered saturated or partially unsaturated heterocyclic ring containing 1-3 heteroatoms independently selected from nitrogen, oxygen, or sulfur; or a fused 5-6 membered heteroaryl ring containing 1-3 heteroatoms independently selected from nitrogen, oxygen, or sulfur; and
each R is independently selected from hydrogen, deuterium, or an optionally substituted group selected from C₁₋₆ aliphatic; a 3-8 membered saturated or partially unsaturated monocyclic carbocyclic ring; phenyl; an 8-10 membered bicyclic aryl ring; a 3-8 membered saturated or partially unsaturated monocyclic heterocyclic ring having 1-4 heteroatoms independently selected from nitrogen, oxygen, or sulfur; a 5-6 membered monocyclic heteroaryl ring having 1-4 heteroatoms independently selected from nitrogen, oxygen, or sulfur; a 6-10 membered bicyclic saturated or partially unsaturated heterocyclic ring having 1-5 heteroatoms independently selected from nitrogen, oxygen, or sulfur; or a 7-10 membered bicyclic heteroaryl ring having 1-5 heteroatoms independently selected from nitrogen, oxygen, or sulfur.

As defined above and described herein, [symbol] is the point of attachment to the amine group. In some embodiments, [symbol] is the point of attachment to the amine group.

As defined above and described herein, # is the point of attachment to the carbinol group. In some embodiments, # is the point of attachment to the carbinol group.

As defined above and described herein, W is independently selected from N, O, S, CU, or CH. In some embodiments, W is N. In some embodiments, W is O. In some embodiments, W is S. In some embodiments, W is CU. In some embodiments, W is CH.

As defined above and described herein, X is independently selected from N, O, S, CU, or CH. In some embodiments, X is N. In some embodiments, X is O. In some embodiments, X is S. In some embodiments, X is CU. In some embodiments, X is CH.

As defined above and described herein, Y is independently selected from N, O, S, CU, or CH. In some embodiments, Y is N. In some embodiments, Y is O. In some embodiments, Y is S. In some embodiments, Y is CU. In some embodiments, Y is CH.

As defined above and described herein, Z is independently selected from N, O, S, CU, or CH. In some embodiments, Z is N. In some embodiments, Z is O. In some embodiments, Z is S. In some embodiments, Z is CU. In some embodiments, Z is CH.

As defined above and described herein, k is 0, 1, 2, 3, or 4. In some embodiments k is O. In some embodiments, k is 1. In some embodiments, k is 2. In some embodiments, k is 3. In some embodiments, k is 4.

As defined above and described herein, each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, U is halogen. In some embodiments, U is fluorine. In some embodiments, U is chlorine. In some embodiments, U is bromine.

In some embodiments, U is —R. In some embodiments, U is hydrogen. In some embodiments, U is deuterium. In some embodiments, U is optionally substituted C₁₋₆ aliphatic. In some embodiments, U is an optionally substituted 3-8 membered saturated or partially unsaturated monocyclic carbocyclic ring. In some embodiments, U is an optionally substituted 8-10 membered bicyclic aryl ring. In some embodiments, U is an optionally substituted 3-8 membered saturated or partially unsaturated monocyclic heterocyclic ring having 1-4 heteroatoms independently selected from nitrogen, oxygen, or sulfur. In some embodiments, U is an optionally substituted 5-6 membered monocyclic heteroaryl ring having 1-4 heteroatoms independently selected from nitrogen, oxygen, or sulfur. In some embodiments, U is an optionally substituted 6-10 membered bicyclic saturated or partially unsaturated heterocyclic ring having 1-5 heteroatoms independently selected from nitrogen, oxygen, or sulfur. In some embodiments, U is an optionally substituted 7-10 membered bicyclic heteroaryl ring having 1-5 heteroatoms independently selected from nitrogen, oxygen, or sulfur.

In some embodiments, U is —S(O)₂R. In some embodiments, U is —S(O)₂CH₃.

In some embodiments, U is an optionally substituted phenyl ring. In some embodiments, U is a phenyl ring, optionally substituted with halogen. In some embodiments, U is a phenyl ring, optionally substituted with fluorine. In some embodiments, U is a phenyl ring, optionally substituted with chlorine.

As defined above and described herein, two occurances of U on adjacent carbon atoms can form an optionally substituted fused ring, selected from a fused phenyl ring; a fused 5-6 membered saturated or partially unsaturated heterocyclic ring containing 1-3 heteroatoms independently selected from nitrogen, oxygen, or sulfur; or a fused 5-6 membered heteroaryl ring containing 1-3 heteroatoms independently selected from nitrogen, oxygen, or sulfur.

In some embodiments, two occurances of U on adjacent carbon atoms form a fused phenyl ring. In some embodiments, two occurances of U on adjacent carbon atoms form an optionally substituted fused phenyl ring. In some embodiments, two occurances of U on adjacent carbon atoms form a fused phenyl ring, optionally substituted with 1 or more halogen atoms. In some embodiments, two occurances of U on adjacent carbon atoms form a fused phenyl ring, optionally substituted with one halogen atom. In some embodiments, two occurances of U on adjacent carbon atoms form a fused phenyl ring, optionally substituted with fluorine. In some embodiments, two occurances of U on adjacent carbon atoms form a fused phenyl ring, optionally substituted with chlorine. In some embodiments, two occurances of U on adjacent carbon atoms form a fused phenyl ring, optionally substituted with 2 halogen atoms. In some embodiments, two occurances of U on adjacent carbon atoms form a fused phenyl ring, optionally substituted with 2 fluorines. In some embodiments, two occurances of U on adjacent carbon atoms form a fused phenyl ring, optionally substituted with 2 chlorines. In some embodiments, two occurances of U on adjacent carbon atoms form a fused phenyl ring, optionally substituted with fluorine and chlorine.

In some embodiments, two occurances of U on adjacent carbon atoms form a fused 5-6 membered heteroaryl ring containing 1-3 heteroatoms independently selected from nitrogen, oxygen, or sulfur. In some embodiments, two occurances of U on adjacent carbon atoms form an optionally substituted fused 5-6 membered heteroaryl ring containing 1-3 heteroatoms independently selected from nitrogen, oxygen, or sulfur.

In some embodiments, two occurances of U on adjacent carbon atoms form a fused 5 membered heteroaryl ring containing 1-3 heteroatoms independently selected from nitrogen, oxygen, or sulfur. In some embodiments, two occurances of U on adjacent carbon atoms form an optionally substituted fused 5-membered heteroaryl ring containing 1-3 heteroatoms independently selected from nitrogen, oxygen, or sulfur.

In some embodiments, two occurances of U on adjacent carbon atoms form a fused 5 membered heteroaryl ring containing one nitrogen and one oxygen heteroatom. In some embodiments, two occurances of U on adjacent carbon atoms form an optionally substituted fused 5 membered heteroaryl ring containing one nitrogen and one oxygen heteroatom. In some embodiments, two occurances of U on adjacent carbon atoms form a fused 5 membered heteroaryl ring containing one nitrogen and one oxygen heteroatom, optionally substituted with phenyl. In some embodiments, two occurances of U on adjacent carbon atoms form a fused 5 membered heteroaryl ring containing one nitrogen and one oxygen heteroatom, optionally substituted with tosyl. In some embodiments, two occurances of U on adjacent carbon atoms form a fused 5 membered heteroaryl ring containing one nitrogen and one oxygen heteroatom, optionally substituted with cyclopropyl.

In some embodiments, two occurances of U on adjacent carbon atoms form a fused 5 membered heteroaryl ring containing one nitrogen and one sulfur heteroatom. In some embodiments, two occurances of U on adjacent carbon atoms form an optionally substituted fused 5 membered heteroaryl ring containing one nitrogen and one sulfur heteroatom. In some embodiments, two occurances of U on adjacent carbon atoms form a fused 5 membered heteroaryl ring containing one nitrogen and one sulfur heteroatom, optionally substituted with phenyl.

In some embodiments, two occurances of U on adjacent carbon atoms form a fused 5-membered heteroaryl ring containing two nitrogen heteroatoms. In some embodiments, two occurances of U on adjacent carbon atoms form an optionally substituted fused 5-membered heteroaryl ring containing two nitrogen heteroatoms. In some embodiments, two occurances of U on adjacent carbon atoms form a fused 5-membered heteroaryl ring containing two nitrogen heteroatoms, optionally substituted with phenyl.

In some embodiments, two occurances of U on adjacent carbon atoms form a fused 6 membered heteroaryl ring containing 1-3 heteroatoms independently selected from nitrogen, oxygen, or sulfur. In some embodiments, two occurances of U on adjacent carbon atoms form an optionally substituted fused 6-membered heteroaryl ring containing 1-3 heteroatoms independently selected from nitrogen, oxygen, or sulfur.

In some embodiments, two occurances of U on adjacent carbon atoms form a fused 6-membered heteroaryl ring containing one nitrogen heteroatom. In some embodiments, two occurances of U on adjacent carbon atoms form an optionally substituted fused 6 membered heteroaryl ring containing one nitrogen heteroatom. In some embodiments, two occurances of U on adjacent carbon atoms form a fused 6-membered heteroaryl ring containing two nitrogen heteroatoms. In some embodiments, two occurances of U on adjacent carbon atoms form an optionally substituted fused 6-membered heteroaryl ring containing two nitrogen heteroatoms.

In some embodiments, the fused ring system formed by two occurances of U on adjacent carbon atoms is quinazolinyl. In some embodiments, the fused ring system formed by two occurances of U on adjacent carbon atoms is an optionally substituted quinazolinyl.

In some embodiments, the fused ring system formed by two occurances of U on adjacent carbon atoms is quinolinyl. In some embodiments, the fused ring system formed by two occurances of U on adjacent carbon atoms is optionally substituted quinolinyl. In some embodiments, the fused ring system formed by two occurances of U on adjacent carbon atoms is quinolinyl, optionally substituted with 1-2 halogen atoms. In some embodiments, the fused ring system formed by two occurances of U on adjacent carbon atoms is quinolinyl, optionally substituted with 1 halogen atom. In some embodiments, the fused ring system formed by two occurances of U on adjacent carbon atoms is quinolinyl, optionally substituted with fluorine. In some embodiments, the fused ring system formed by two occurances of U on adjacent carbon atoms quinolinyl, optionally substituted with chlorine.

In some embodiments, the fused ring system formed by two occurances of U on adjacent carbon atoms is benzoxazolyl. In some embodiments, the fused ring system formed by two occurances of U on adjacent carbon atoms is optionally substituted benzoxazolyl. In some embodiments, the fused ring system formed by two occurances of U on adjacent carbon atoms is benzoxazolyl, optionally substituted with phenyl. In some embodiments, the fused ring system formed by two occurances of U on adjacent carbon atoms is benzoxazolyl, optionally substituted with phenyl and a halogen atom. In some embodiments, the fused ring system formed by two occurances of U on adjacent carbon atoms is benzoxazolyl, optionally substituted with phenyl and chlorine. In some embodiments, the fused ring system formed by two occurances of U on adjacent carbon atoms is benzoxazolyl, optionally substituted with tosyl and chlorine.

In some embodiments, the fused ring system formed by two occurances of U on adjacent carbon atoms is benzisoxazolyl. In some embodiments, the fused ring system formed by two occurances of U on adjacent carbon atoms is optionally substituted benzisoxazolyl. In some embodiments, the fused ring system formed by two occurances of U on adjacent carbon atoms is benzisoxazolyl, optionally substituted with phenyl. In some embodiments, the fused ring system formed by two occurances of U on adjacent carbon atoms is benzisoxazolyl, optionally substituted with cyclopropyl and a halogen atom. In some embodiments, the fused ring system formed by two occurances of U on adjacent carbon atoms is benzisoxazolyl, optionally substituted with cyclopropyl and chlorine.

In some embodiments, the fused ring system formed by two occurances of U on adjacent carbon atoms is benzothiazolyl. In some embodiments, the fused ring system formed by two occurances of U on adjacent carbon atoms is optionally substituted benzothiazolyl. In some embodiments, the fused ring system formed by two occurances of U on adjacent carbon atoms is benzothiazolyl, optionally substituted with phenyl.

In some embodiments, the fused ring system formed by two occurances of U on adjacent carbon atoms is benzisothiazolyl. In some embodiments, the fused ring system formed by two occurances of U on adjacent carbon atoms is optionally substituted benzisothiazolyl. In some embodiments, the fused ring system formed by two occurances of U on adjacent carbon atoms is benzisothiazolyl, optionally substituted with phenyl.

In some embodiments, the fused ring system formed by two occurances of U on adjacent carbon atoms is benzimidazolyl. In some embodiments, the fused ring system formed by two occurances of U on adjacent carbon atoms is optionally substituted benzimidazolyl. In some embodiments, the fused ring system formed by two occurances of U on adjacent carbon atoms is benzimidazolyl, optionally substituted with phenyl.

In some embodiments, W, X, Y, and Z provide a phenyl ring. In some embodiments, W, X, Y, and Z provide a phenyl ring, substituted with k occurances of U.

In some embodiments, W, X, Y, and Z provide a pyridinyl ring. In some embodiments, W, X, Y, and Z provide a pyridinyl ring, substituted with k occurances of U.

In some embodiments, one or more of W, X, Y, or Z are CH; and k is O. In some embodiments, one or more of W, X, or Y are CH; Z is N; and k is 0.

In some embodiments, one or more of W, X, Y, or Z are CH; k is 1; and U is halogen. In some embodiments, one or more of W, X, Y, and Z are CH; k is 1; and U is fluorine. In some embodiments, one or more of W, X, Y, and Z are CH; k is 1; and U is chlorine. In some embodiments, one or more of W, X, Y, and Z are CH; k is 1; and U is bromine.

In some embodiments, one or more of W, X, and Y are CH; Z is N; k is 1; and U optionally substituted phenyl. In some embodiments, one or more of W, X, and Y are CH; Z is N; k is 1; and U is phenyl, optionally substituted with halogen. In some embodiments, one or more of W, X, and Y are CH; Z is N; k is 1; and U is phenyl, optionally substituted with fluorine.

In some embodiments, one or more of W is N; X, Y, and Z are CH; k is 1; and U is optionally substituted phenyl. In some embodiments, one or more of W is N; X, Y, and Z are CH; k is 1; and U is phenyl, optionally substituted with halogen. In some embodiments, one or more of W is N; X, Y, and Z are CH; k is 1; and U is phenyl, optionally substituted with fluorine.

In some embodiments, one or more of W, X, and Y are CH; Z is N; k is 2; and the two occurances of U on adjacent carbon atoms form a fused phenyl ring. In some embodiments, one or more of W, X, and Y are CH; Z is N; k is 2; and the two occurances of U on adjacent carbon atoms form an optionally substituted fused phenyl ring. In some embodiments, one or more of W, X, and Y are CH; Z is N; k is 2; and the two occurances of U on adjacent carbon atoms form a fused phenyl ring, optionally substituted with halogen. In some embodiments, one or more of W, X, and Y are CH; Z is N; k is 2; and the two occurances of U on adjacent carbon atoms form a fused phenyl ring, optionally substituted with chlorine.

In some embodiments, one or more of W is N; X, Y, and Z are CH; k is 2; and the two occurances of U on adjacent carbon atoms form a fused phenyl ring. In some embodiments, one or more of W is N; X, Y, and Z are CH; k is 2; and the two occurances of U on adjacent carbon atoms form an optionally substituted fused phenyl ring. In some embodiments, one or more of W is N; X, Y, and Z are CH; k is 2; and the two occurances of U on adjacent carbon atoms form a fused phenyl ring, optionally substituted with halogen. In some embodiments, one or more of W is N; X, Y, and Z are CH; k is 2; and the two occurances of U on adjacent carbon atoms form a fused phenyl ring, optionally substituted with fluorine. In some embodiments, one or more of W is N; X, Y, and Z are CH; k is 2; and the two occurances of U on adjacent carbon atoms form a fused phenyl ring, optionally substituted with chlorine. In some embodiments, one or more of W is N; X, Y, and Z are CH; k is 2; and the two occurances of U on adjacent carbon atoms form a fused phenyl ring, optionally substituted with chlorine and fluorine. In some embodiments, one or more of W is N; X, Y, and Z are CH; k is 2; and the two occurances of U on adjacent carbon atoms form a fused phenyl ring, optionally substituted with chlorine at 2 positions.

In some embodiments, one or more of W, X, Y, and Z are CH; k is 2; and the two occurances of U on adjacent carbon atoms form a fused 5-6 membered heteroaryl ring containing 1-3 heteroatoms independently selected from nitrogen, oxygen, or sulfur. In some embodiments, one or more of W, X, Y, and Z are CH; k is 2; and the two occurances of U on adjacent carbon atoms form an optionally substituted fused 5-6 membered heteroaryl ring containing 1-3 heteroatoms independently selected from nitrogen, oxygen, or sulfur.

In some embodiments, one or more of W, X, Y, and Z are CH; k is 2; and the two occurances of U on adjacent carbon atoms form an optionally substituted fused 6 membered heteroaryl ring containing one nitrogen heteroatom. In some embodiments, one or more of W, X, Y, and Z are CH; k is 2; and the two occurances of U on adjacent carbon atoms form a fused pyridine ring. In some embodiments, one or more of W, X, Y, and Z are CH; k is 2; and the two occurances of U on adjacent carbon atoms form an optionally substituted fused pyridine ring. In some embodiments, one or more of W, X, Y, and Z are CH; k is 2; and the two occurances of U on adjacent carbon atoms form an optionally substituted fused 6 membered heteroaryl ring containing two nitrogen heteroatoms. In some embodiments, one or more of W, X, Y, and Z are CH; k is 2; and the two occurances of U on adjacent carbon atoms form a fused pyrimidine ring. In some embodiments, one or more of W, X, Y, and Z are CH; k is 2; and the two occurances of U on adjacent carbon atoms form an optionally substituted fused pyrimidine ring.

In some embodiments, one or more of W, X, Y, and Z are CH; k is 2; and the two occurances of U on adjacent carbon atoms form fused aryl ring with 2 heteroatoms. In some embodiments, one or more of W, X, Y, and Z are CH; k is 2; and the two occurances of U on adjacent carbon atoms form a 5 membered fused oxazole ring. In some embodiments, one or more of W, X, Y, and Z are CH; k is 2; and the two occurances of U on adjacent carbon atoms form a 5 membered fused oxazole ring, optionally substituted with phenyl.

In some embodiments, one or more of W, X, Y, and Z is CH; k is 2; and the two occurances of U on adjacent carbon atoms form an optionally substituted fused 5 membered heteroaryl ring containing one nitrogen and one oxygen heteroatom. In some embodiments, one or more of W, X, Y, and Z is CH; k is 2; and the two occurances of U on adjacent carbon atoms form a fused 5 membered heteroaryl ring containing one nitrogen and one oxygen heteroatoms, optionally substituted with phenyl. In some embodiments, one or more of W, X, Y, and Z is CH; k is 2; and the two occurances of U on adjacent carbon atoms form a fused 5 membered heteroaryl ring containing one nitrogen and one oxygen heteroatoms, optionally substituted with tosyl. In some embodiments, one or more of W, X, Y, and Z is CH; k is 2; and the two occurances of U on adjacent carbon atoms form a fused 5 membered heteroaryl ring containing one nitrogen and one oxygen heteroatoms, optionally substituted with cyclopropyl.

In some embodiments, one or more of W, X, Y, and Z are CH; k is 2; and the two occurances of U on adjacent carbon atoms form an optionally substituted fused oxazole ring. In some embodiments, one or more of W, X, Y, and Z are CH; k is 2; and the two occurances of U on adjacent carbon atoms form a fused oxazole ring, optionally substituted with phenyl. In some embodiments, one or more of W, X, Y, and Z are CH; k is 2; and the two occurances of U on adjacent carbon atoms form a fused oxazole ring, optionally substituted with tosyl.

In some embodiments, one or more of W, X, Y, and Z are CH; k is 2; and the two occurances of U on adjacent carbon atoms form an optionally substituted fused isoxazole ring. In some embodiments, one or more of W, X, Y, and Z are CH; k is 2; and the two occurances of U on adjacent carbon atoms form a fused isoxazole ring, optionally substituted with phenyl. In some embodiments, one or more of W, X, Y, and Z are CH; k is 2; and the two occurances of U on adjacent carbon atoms form a fused isoxazole ring, optionally substituted with cyclopropyl.

In some embodiments, one or more of W, X, Y, and Z are CH; k is 2; and the two occurances of U on adjacent carbon atoms form an optionally substituted fused 5 membered heteroaryl ring containing one nitrogen and one sulfur heteroatom. In some embodiments, one or more of W, X, Y, and Z is CH; k are 2; and the two occurances of U on adjacent carbon atoms form a fused 5 membered heteroaryl ring containing one nitrogen and one sulfur heteroatom, optionally substituted by phenyl.

In some embodiments, one or more of W, X, Y, and Z are CH; k is 2; and the two occurances of U on adjacent carbon atoms form an optionally substituted fused thiazole ring. In some embodiments, one or more of W, X, Y, and Z are CH; k is 2; and the two occurances of U on adjacent carbon atoms form a fused thiazole ring, optionally substituted with phenyl.

In some embodiments, one or more of W, X, Y, and Z are CH; k is 2; and the two occurances of U on adjacent carbon atoms form an optionally substituted fused 5 membered heteroaryl ring containing two nitrogen heteroatoms. In some embodiments, one or more of W, X, Y, and Z are CH; k is 2; and the two occurances of U on adjacent carbon atoms form an optionally substituted fused imidazole ring. In some embodiments, one or more of W, X, Y, and Z are CH; k is 2; and the two occurances of U on adjacent carbon atoms form a fused imidazole ring, optionally substituted with phenyl.

In some embodiments, one or more of W, X, Y, and Z are CH; k is 3; U₁ is chlorine and U₂ and U₃ on adjacent carbon atoms form an optionally substituted fused 5 membered heteroaryl ring containing one nitrogen and one oxygen heteroatom. In some embodiments, one or more of W, X, Y, and Z are CH; k is 3; U₁ is chlorine and U₂ and U₃ on adjacent carbon atoms form a fused 5 membered heteroaryl ring containing one nitrogen and one oxygen heteroatom, optionally substituted with phenyl. In some embodiments, one or more of W, X, Y, and Z are CH; k is 3; U₁ is chlorine and U₂ and U₃ on adjacent carbon atoms form a fused 5 membered heteroaryl ring containing one nitrogen and one oxygen heteroatom, optionally substituted with tosyl.

In some embodiments, one or more of W, X, Y, and Z are CH; k is 3; U₁ is chlorine and U₂ and U₃ on adjacent carbon atoms form an optionally substituted fused oxazole ring. In some embodiments, one or more of W, X, Y, and Z are CH; k is 3; U₁ is chlorine and U₂ and U₃ on adjacent carbon atoms form a fused oxazole ring, optionally substituted with phenyl. In some embodiments, one or more of W, X, Y, and Z are CH; k is 3; U₁ is chlorine and U₂ and U₃ on adjacent carbon atoms form a fused oxazole ring, optionally substituted with tosyl.

In some embodiments, one or more of W, X, Y, and Z are CH; k is 3; U₁ is chlorine and U₂ and U₃ on adjacent carbon atoms form an optionally substituted fused isoxazole ring. In some embodiments, one or more of W, X, Y, and Z are CH; k is 3; U₁ is chlorine and U₂ and U₃ adjacent carbon atoms form a fused isoxazole ring, optionally substituted with cyclopropyl.

As defined above and described herein, each R is independently selected from hydrogen, deuterium, or an optionally substituted group selected from C₁₋₆ aliphatic; a 3-8 membered saturated or partially unsaturated monocyclic carbocyclic ring; phenyl; an 8-10 membered bicyclic aryl ring; a 3-8 membered saturated or partially unsaturated monocyclic heterocyclic ring having 1-4 heteroatoms independently selected from nitrogen, oxygen, or sulfur; a 5-6 membered monocyclic heteroaryl ring having 1-4 heteroatoms independently selected from nitrogen, oxygen, or sulfur; a 6-10 membered bicyclic saturated or partially unsaturated heterocyclic ring having 1-5 heteroatoms independently selected from nitrogen, oxygen, or sulfur; or a 7-10 membered bicyclic heteroaryl ring having 1-5 heteroatoms independently selected from nitrogen, oxygen, or sulfur.

In some embodiments, R is hydrogen. In some embodiments, R is deuterium. In some embodiments, R is C₁₋₆ aliphatic. In some embodiments R is methyl. In some embodiments, R is ethyl. In some embodiments, R is optionally substituted C₁₋₆ aliphatic. In some embodiments, R is optionally substituted methyl. In some embodiments, R is optionally substituted ethyl. In some embodiments, R is phenyl. In some embodiments, R is optionally substituted phenyl. In some embodiments, R is phenyl, optionally substituted with halogen. In some embodiments, R is phenyl, optionally substituted with fluorine.

In some embodiments, the present invention provides an aldehyde trap compound of formula V:

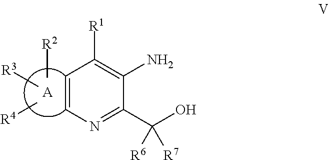

or a pharmaceutically acceptable salt thereof, wherein:
Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom; or a 6-membered partially unsaturated heterocyclic or heteroaromatic ring containing 1-3 heteroatoms independently selected from nitrogen, oxygen, or sulfur; or a 7-membered partially unsaturated heterocyclic or heteroaromatic ring containing 1-3 heteroatoms independently selected from nitrogen, oxygen, or sulfur;
R¹ is H, D, halogen, —CN, —OR, —SR, or optionally substituted C₁₋₆ aliphatic;
each R is independently selected from hydrogen, deuterium, or an optionally substituted group selected from: C₁₋₆ aliphatic, a 3-8 membered saturated or partially unsaturated monocyclic carbocyclic ring, phenyl, an 8-10 membered bicyclic aryl ring, a 3-8 membered saturated or partially unsaturated monocyclic heterocyclic ring having 1-4 heteroatoms independently selected from nitrogen, oxygen, or sulfur, a 5-6 membered monocyclic heteroaryl ring having 1-4 heteroatoms independently selected from nitrogen, oxygen, or sulfur, a 6-10 membered bicyclic saturated or partially unsaturated heterocyclic ring having 1-5 heteroatoms independently selected from nitrogen, oxygen, or sulfur, or a 7-10 membered bicyclic heteroaryl ring having 1-5 heteroatoms independently selected from nitrogen, oxygen, or sulfur;
R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms; or R⁶ and R⁷, taken together with the carbon atom to which they are attached, form a 3-8 membered cycloalkyl or heterocyclyl ring containing 1-2 heteroatoms selected from nitrogen, oxygen, and sulfur.

As defined generally above, Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom; or a 6-membered partially unsaturated heterocyclic or heteroaromatic ring containing 1-3 heteroatoms independently selected from nitrogen, oxygen, or sulfur; or a 7-membered partially unsaturated heterocyclic or heteroaromatic ring containing 1-3 heteroatoms independently selected from nitrogen, oxygen, or sulfur.

In some embodiments, Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom. In some embodiments, Ring A is a 6-membered partially unsaturated heterocyclic or heteroaromatic ring containing 1-3 heteroatoms independently selected from nitrogen, oxygen, or sulfur. In some embodiments, Ring A is a 7-membered partially unsaturated heterocyclic or heteroaromatic ring containing 1-3 heteroatoms independently selected from nitrogen, oxygen, or sulfur.

In some embodiments, Ring A is imidazole or triazole. In some embodiments, Ring A is thiazole. In some embodiments, Ring A is thiophene or furan. In some embodiments, Ring A is pyridine, pyrimidine, pyrazine, pyridazine, or 1,2,4-triazine. In some embodiments, Ring A is pyridine.

As defined generally above, R¹ is H, D, halogen, —CN, —OR, —SR, or optionally substituted C₁₋₆ aliphatic.

In some embodiments, R¹ is H. In some embodiments, R¹ is D. In some embodiments, R¹ is halogen. In some embodiments, R¹ is —CN. In some embodiments, R¹ is —OR. In some embodiments, R¹ is —SR. In some embodiments, R¹ is optionally substituted C₁₋₆ aliphatic.

As described generally above, R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R² is absent. In some embodiments, R² is —R. In some embodiments, R² is halogen. In some embodiments, R² is —CN. In some embodiments, R² is —OR. In some embodiments, R² is —SR. In some embodiments, R² is —N(R)₂. In some embodiments, R² is —N(R)C(O)R. In some embodiments, R² is —C(O)N(R)₂. In some embodiments, R² is —N(R)C(O)N(R)₂. In some embodiments, R² is —N(R)C(O)OR. In some embodiments, R² is —OC(O)N(R)₂. In some embodiments, R² is —N(R)S(O)₂R. In some embodiments, R² is —SO₂N(R)₂. In some embodiments, R² is —C(O)R. In some embodiments, R² is —C(O)OR. In some embodiments, R² is —OC(O)R. In some embodiments, R² is —S(O)R. In some embodiments, R² is —S(O)₂R.

In some embodiments, R² is hydrogen. In some embodiments, R² is deuterium. In some embodiments, R² is an optionally substituted C₁₋₆ aliphatic. In some embodiments, R² is an optionally substituted 3-8 membered saturated or partially unsaturated monocyclic carbocyclic ring. In some embodiments, R² is an optionally substituted phenyl. In some embodiments, R² is an optionally substituted 8-10 membered bicyclic aryl ring. In some embodiments, R² is an optionally substituted 3-8 membered saturated or partially unsaturated monocyclic heterocyclic ring having 1-4 heteroatoms independently selected from nitrogen, oxygen, or sulfur. In some embodiments, R² is an optionally substituted 5-6 membered monocyclic heteroaryl ring having 1-4 heteroatoms independently selected from nitrogen, oxygen, or sulfur. In some embodiments, R² is an optionally substituted 6-10 membered bicyclic saturated or partially unsaturated heterocyclic ring having 1-5 heteroatoms independently selected from nitrogen, oxygen, or sulfur. In some embodiments, R² is an optionally substituted 7-10 membered bicyclic heteroaryl ring having 1-5 heteroatoms independently selected from nitrogen, oxygen, or sulfur.

In some embodiments, R² is Cl or Br. In some embodiments, R² is Cl.

As defined generally above, R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R³ is absent. In some embodiments, R³ is —R. In some embodiments, R³ is halogen. In some embodiments, R³ is —CN. In some embodiments, R³ is —OR. In some embodiments, R³ is —SR. In some embodiments, R³ is —N(R)₂. In some embodiments, R³ is —N(R)C(O)R. In some embodiments, R³ is —C(O)N(R)₂. In some embodiments, R³ is —N(R)C(O)N(R)₂. In some embodiments, R³ is —N(R)C(O)OR. In some embodiments, R³ is —OC(O)N(R)₂. In some embodiments, R³ is —N(R)S(O)₂R. In some embodiments, R³ is —SO₂N(R)₂. In some embodiments, R³ is —C(O)R. In some embodiments, R³ is —C(O)OR. In some embodiments, R³ is —OC(O)R. In some embodiments, R³ is —S(O)R. In some embodiments, R³ is —S(O)₂R.

In some embodiments, R³ is hydrogen. In some embodiments, R³ is deuterium. In some embodiments, R³ is an optionally substituted C₁₋₆ aliphatic. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aryl ring. In some embodiments, R³ is an optionally substituted 3-8 membered saturated or partially unsaturated monocyclic heterocyclic ring having 1-4 heteroatoms independently selected from nitrogen, oxygen, or sulfur. In some embodiments, R³ is an optionally substituted 5-6 membered monocyclic heteroaryl ring having 1-4 heteroatoms independently selected from nitrogen, oxygen, or sulfur. In some embodiments, R³ is an optionally substituted 6-10 membered bicyclic saturated or partially unsaturated heterocyclic ring having 1-5 heteroatoms independently selected from nitrogen, oxygen, or sulfur. In some embodiments, R³ is an optionally substituted 7-10 membered bicyclic heteroaryl ring having 1-5 heteroatoms independently selected from nitrogen, oxygen, or sulfur.

In some embodiments, R³ is Cl or Br. In some embodiments, R³ is Cl.

As defined generally above, R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R⁴ is absent. In some embodiments, R⁴ is —R. In some embodiments, R⁴ is halogen. In some embodiments, R⁴ is —CN. In some embodiments, R⁴ is —OR. In some embodiments, R⁴ is —SR. In some embodiments, R⁴ is —N(R)₂. In some embodiments, R⁴ is —N(R)C(O)R. In some embodiments, R⁴ is —C(O)N(R)₂. In some embodiments, R⁴ is —N(R)C(O)N(R)₂. In some embodiments, R⁴ is —N(R)C(O)OR. In some embodiments, R⁴ is —OC(O)N(R)₂. In some embodiments, R⁴ is —N(R)S(O)₂R. In some embodiments, R⁴ is —SO₂N(R)₂. In some embodiments, R⁴ is —C(O)R. In some embodiments, R⁴ is —C(O)OR. In some embodiments, R⁴ is —OC(O)R. In some embodiments, R⁴ is —S(O)R. In some embodiments, R⁴ is —S(O)₂R.

In some embodiments, R⁴ is hydrogen. In some embodiments, R⁴ is deuterium. In some embodiments, R⁴ is an optionally substituted C₁₋₆ aliphatic. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aryl ring. In some embodiments, R⁴ is an optionally substituted 3-8 membered saturated or partially unsaturated monocyclic heterocyclic ring having 1-4 heteroatoms independently selected from nitrogen, oxygen, or sulfur. In some embodiments, R⁴ is an optionally substituted 5-6 membered monocyclic heteroaryl ring having 1-4 heteroatoms independently selected from nitrogen, oxygen, or sulfur. In some embodiments, R⁴ is an optionally substituted 6-10 membered bicyclic saturated or partially unsaturated heterocyclic ring having 1-5 heteroatoms independently selected from nitrogen, oxygen, or sulfur. In some embodiments, R⁴ is an optionally substituted 7-10 membered bicyclic heteroaryl ring having 1-5 heteroatoms independently selected from nitrogen, oxygen, or sulfur.

In some embodiments, R⁴ is Cl or Br. In some embodiments, R⁴ is Cl.

As described generally above, R⁶ is C₁₋₄ aliphatic optionally substituted with 1, 2, or 3 deuterium or halogen atoms.

In some embodiments, R⁶ is C₁₋₄ aliphatic. In some embodiments, R⁶ is C₁₋₄ aliphatic optionally substituted with 1, 2, or 3 deuterium atoms. In some embodiments, R⁶ is C₁₋₄ aliphatic optionally substituted with 1, 2, or 3 halogen atoms.

In some embodiments, R⁶ is C₁₋₄ alkyl. In some embodiments, R⁶ is C₁₋₄ alkyl optionally substituted with 1, 2, or 3 deuterium or halogen atoms. In some embodiments, R⁶ is C₁₋₄ alkyl optionally substituted with 1, 2, or 3 halogen atoms. In some embodiments, R⁶ is methyl or ethyl optionally substituted with 1, 2, or 3 halogen atoms. In some embodiments, R⁶ is methyl.

As defined generally above, R⁷ is C₁₋₄ aliphatic optionally substituted with 1, 2, or 3 deuterium or halogen atoms.

In some embodiments, R⁷ is C₁₋₄ aliphatic. In some embodiments, R⁷ is C₁₋₄ aliphatic optionally substituted with 1, 2, or 3 deuterium atoms. In some embodiments, R⁷ is C₁₋₄ aliphatic optionally substituted with 1, 2, or 3 halogen atoms.

In some embodiments, R⁷ is C₁₋₄ alkyl. In some embodiments, R⁷ is C₁₋₄ alkyl optionally substituted with 1, 2, or 3 deuterium or halogen atoms. In some embodiments, R⁷ is C₁₋₄ alkyl optionally substituted with 1, 2, or 3 halogen atoms. In some embodiments, R⁷ is methyl or ethyl optionally substituted with 1, 2, or 3 halogen atoms. In some embodiments, R⁷ is methyl.

As defined generally above, in some embodiments, R⁶ and R⁷, taken together with the carbon atom to which they are attached, form a 3-8 membered cycloalkyl or heterocyclyl ring containing 1-2 heteroatoms selected from nitrogen, oxygen, and sulfur.

In some embodiments, R⁶ and R⁷, taken together with the carbon atom to which they are attached, form a 3-8 membered cycloalkyl. In some embodiments, R⁶ and R⁷, taken together with the carbon atom to which they are attached, form a 3-8 membered heterocyclyl ring containing 1-2 heteroatoms selected from nitrogen, oxygen, and sulfur.

In some embodiments, R⁶ and R⁷, taken together with the carbon atom to which they are attached, form a cyclopropyl, cyclobutyl, or cyclopentyl ring. In some embodiments, R⁶ and R⁷, taken together with the carbon atom to which they are attached, form an oxirane, oxetane, tetrahydrofuran, or aziridine.

In some embodiments, R⁶ and R⁷ are methyl.

In another aspect, the present invention provides an aldehyde trap compound of formula VI:

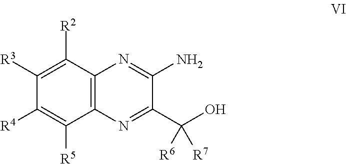

or a pharmaceutically acceptable salt thereof, wherein:
R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
each R is independently selected from hydrogen, deuterium, or an optionally substituted group selected from: C₁₋₆ aliphatic, a 3-8 membered saturated or partially unsaturated monocyclic carbocyclic ring, phenyl, an 8-10 membered bicyclic aryl ring, a 3-8 membered saturated or partially unsaturated monocyclic heterocyclic ring having 1-4 heteroatoms independently selected from nitrogen, oxygen, or sulfur, a 5-6 membered monocyclic heteroaryl ring having 1-4 heteroatoms independently selected from nitrogen, oxygen, or sulfur, a 6-10 membered bicyclic saturated or partially unsaturated heterocyclic ring having 1-5 heteroatoms independently selected from nitrogen, oxygen, or sulfur, or a 7-10 membered bicyclic heteroaryl ring having 1-5 heteroatoms independently selected from nitrogen, oxygen, or sulfur;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms; or R⁶ and R⁷, taken together with the carbon atom to which they are attached, form a 3-8 membered cycloalkyl or heterocyclyl ring containing 1-2 heteroatoms selected from nitrogen, oxygen, and sulfur.

As described generally above, R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R² is —R. In some embodiments, R² is halogen. In some embodiments, R² is —CN. In some embodiments, R² is —OR. In some embodiments, R² is —SR. In some embodiments, R² is —N(R)₂. In some embodiments, R² is —N(R)C(O)R. In some embodiments, R² is —C(O)N(R)₂. In some embodiments, R² is —N(R)C(O)N(R)₂. In some embodiments, R² is —N(R)C(O)OR. In some embodiments, R² is —OC(O)N(R)₂. In some embodiments, R² is —N(R)S(O)₂R. In some embodiments, R² is —SO₂N(R)₂. In some embodiments, R² is —C(O)R. In some embodiments, R² is —C(O)OR. In some embodiments, R² is —OC(O)R. In some embodiments, R² is —S(O)R. In some embodiments, R² is —S(O)₂R.

In some embodiments, R² is hydrogen. In some embodiments, R² is deuterium. In some embodiments, R² is an optionally substituted C₁₋₆ aliphatic. In some embodiments, R² is an optionally substituted 3-8 membered saturated or partially unsaturated monocyclic carbocyclic ring. In some embodiments, R² is an optionally substituted phenyl. In some embodiments, R² is an optionally substituted 8-10 membered bicyclic aryl ring. In some embodiments, R² is an optionally substituted 3-8 membered saturated or partially unsaturated monocyclic heterocyclic ring having 1-4 heteroatoms independently selected from nitrogen, oxygen, or sulfur. In some embodiments, R² is an optionally substituted 5-6 membered monocyclic heteroaryl ring having 1-4 heteroatoms independently selected from nitrogen, oxygen, or sulfur. In some embodiments, R² is an optionally substituted 6-10 membered bicyclic saturated or partially unsaturated heterocyclic ring having 1-5 heteroatoms independently selected from nitrogen, oxygen, or sulfur. In some embodiments, R² is an optionally substituted 7-10 membered bicyclic heteroaryl ring having 1-5 heteroatoms independently selected from nitrogen, oxygen, or sulfur.

In some embodiments, R² is Cl or Br. In some embodiments, R² is Cl.

As defined generally above, R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R³ is —R. In some embodiments, R³ is halogen. In some embodiments, R³ is —CN. In some embodiments, R³ is —OR. In some embodiments, R³ is —SR. In some embodiments, R³ is —N(R)₂. In some embodiments, R³ is —N(R)C(O)R. In some embodiments, R³ is —C(O)N(R)₂. In some embodiments, R³ is —N(R)C(O)N(R)₂. In some embodiments, R³ is —N(R)C(O)OR. In some embodiments, R³ is —OC(O)N(R)₂. In some embodiments, R³ is —N(R)S(O)₂R. In some embodiments, R³ is —SO₂N(R)₂. In some embodiments, R³ is —C(O)R. In some embodiments, R³ is —C(O)OR. In some embodiments, R³ is —OC(O)R. In some embodiments, R³ is —S(O)R. In some embodiments, R³ is —S(O)₂R.

In some embodiments, R³ is hydrogen. In some embodiments, R³ is deuterium. In some embodiments, R³ is an optionally substituted C₁₋₆ aliphatic. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aryl ring. In some embodiments, R³ is an optionally substituted 3-8 membered saturated or partially unsaturated monocyclic heterocyclic ring having 1-4 heteroatoms independently selected from nitrogen, oxygen, or sulfur. In some embodiments, R³ is an optionally substituted 5-6 membered monocyclic heteroaryl ring having 1-4 heteroatoms independently selected from nitrogen, oxygen, or sulfur. In some embodiments, R³ is an optionally substituted 6-10 membered bicyclic saturated or partially unsaturated heterocyclic ring having 1-5 heteroatoms independently selected from nitrogen, oxygen, or sulfur. In some embodiments, R³ is an optionally substituted 7-10 membered bicyclic heteroaryl ring having 1-5 heteroatoms independently selected from nitrogen, oxygen, or sulfur.

In some embodiments, R³ is Cl or Br. In some embodiments, R³ is Cl.

As defined generally above, R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R⁴ is —R. In some embodiments, R⁴ is halogen. In some embodiments, R⁴ is —CN. In some embodiments, R⁴ is —OR. In some embodiments, R⁴ is —SR. In some embodiments, R⁴ is —N(R)₂. In some embodiments, R⁴ is —N(R)C(O)R. In some embodiments, R⁴ is —C(O)N(R)₂. In some embodiments, R⁴ is —N(R)C(O)N(R)₂. In some embodiments, R⁴ is —N(R)C(O)OR. In some embodiments, R⁴ is —OC(O)N(R)₂. In some embodiments, R⁴ is —N(R)S(O)₂R. In some embodiments, R⁴ is —SO₂N(R)₂. In some embodiments, R⁴ is —C(O)R. In some embodiments, R⁴ is —C(O)OR. In some embodiments, R⁴ is —OC(O)R. In some embodiments, R⁴ is —S(O)R. In some embodiments, R⁴ is —S(O)₂R.

In some embodiments, R⁴ is hydrogen. In some embodiments, R⁴ is deuterium. In some embodiments, R⁴ is an optionally substituted C₁₋₆ aliphatic. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aryl ring. In some embodiments, R⁴ is an optionally substituted 3-8 membered saturated or partially unsaturated monocyclic heterocyclic ring having 1-4 heteroatoms independently selected from nitrogen, oxygen, or sulfur. In some embodiments, R⁴ is an optionally substituted 5-6 membered monocyclic heteroaryl ring having 1-4 heteroatoms independently selected from nitrogen, oxygen, or sulfur. In some embodiments, R⁴ is an optionally substituted 6-10 membered bicyclic saturated or partially unsaturated heterocyclic ring having 1-5 heteroatoms independently selected from nitrogen, oxygen, or sulfur. In some embodiments, R⁴ is an optionally substituted 7-10 membered bicyclic heteroaryl ring having 1-5 heteroatoms independently selected from nitrogen, oxygen, or sulfur.

In some embodiments, R⁴ is Cl or Br. In some embodiments, R⁴ is Cl.

As defined generally above, R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R⁵ is —R. In some embodiments, R⁵ is halogen. In some embodiments, R⁵ is —CN. In some embodiments, R⁵ is —OR. In some embodiments, R⁵ is —SR. In some embodiments, R⁵ is —N(R)₂. In some embodiments, R⁵ is —N(R)C(O)R. In some embodiments, R⁵ is —C(O)N(R)₂. In some embodiments, R⁵ is —N(R)C(O)N(R)₂. In some embodiments, R⁵ is —N(R)C(O)OR. In some embodiments, R⁵ is —OC(O)N(R)₂. In some embodiments, R⁵ is —N(R)S(O)₂R. In some embodiments, R⁵ is —SO₂N(R)₂. In some embodiments, R⁵ is —C(O)R. In some embodiments, R⁵ is —C(O)OR. In some embodiments, R⁵ is —OC(O)R. In some embodiments, R⁵ is —S(O)R. In some embodiments, R⁵ is —S(O)₂R.

In some embodiments, R⁵ is hydrogen. In some embodiments, R⁵ is deuterium. In some embodiments, R⁵ is an optionally substituted C₁₋₆ aliphatic. In some embodiments, R⁵ is an optionally substituted 3-8 membered saturated or partially unsaturated monocyclic carbocyclic ring. In some embodiments, R⁵ is an optionally substituted phenyl. In some embodiments, R⁵ is an optionally substituted 8-10 membered bicyclic aryl ring. In some embodiments, R⁵ is an optionally substituted 3-8 membered saturated or partially unsaturated monocyclic heterocyclic ring having 1-4 heteroatoms independently selected from nitrogen, oxygen, or sulfur. In some embodiments, R⁵ is an optionally substituted 5-6 membered monocyclic heteroaryl ring having 1-4 heteroatoms independently selected from nitrogen, oxygen, or sulfur. In some embodiments, R⁵ is an optionally substituted 6-10 membered bicyclic saturated or partially unsaturated heterocyclic ring having 1-5 heteroatoms independently selected from nitrogen, oxygen, or sulfur. In some embodiments, R⁵ is an optionally substituted 7-10 membered bicyclic heteroaryl ring having 1-5 heteroatoms independently selected from nitrogen, oxygen, or sulfur.

In some embodiments, R⁵ is Cl or Br. In some embodiments, R⁵ is Cl.

As described generally above, R⁶ is C₁₋₄ aliphatic optionally substituted with 1, 2, or 3 deuterium or halogen atoms.

In some embodiments, R⁶ is C₁₋₄ aliphatic. In some embodiments, R⁶ is C₁₋₄ aliphatic optionally substituted with 1, 2, or 3 deuterium atoms. In some embodiments, R⁶ is C₁₋₄ aliphatic optionally substituted with 1, 2, or 3 halogen atoms.

In some embodiments, R⁶ is C₁₋₄ alkyl. In some embodiments, R⁶ is C₁₋₄ alkyl optionally substituted with 1, 2, or 3 deuterium or halogen atoms. In some embodiments, R⁶ is C₁₋₄ alkyl optionally substituted with 1, 2, or 3 halogen atoms. In some embodiments, R⁶ is methyl or ethyl optionally substituted with 1, 2, or 3 halogen atoms. In some embodiments, R⁶ is methyl.

As defined generally above, R⁷ is C₁₋₄ aliphatic optionally substituted with 1, 2, or 3 deuterium or halogen atoms.

In some embodiments, R⁷ is C₁₋₄ aliphatic. In some embodiments, R⁷ is C₁₋₄ aliphatic optionally substituted with 1, 2, or 3 deuterium atoms. In some embodiments, R⁷ is C₁₋₄ aliphatic optionally substituted with 1, 2, or 3 halogen atoms.

In some embodiments, R⁷ is C₁₋₄ alkyl. In some embodiments, R⁷ is C₁₋₄ alkyl optionally substituted with 1, 2, or 3 deuterium or halogen atoms. In some embodiments, R⁷ is C₁₋₄ alkyl optionally substituted with 1, 2, or 3 halogen atoms. In some embodiments, R⁷ is methyl or ethyl optionally substituted with 1, 2, or 3 halogen atoms. In some embodiments, R⁷ is methyl.

As defined generally above, in some embodiments, R⁶ and R⁷, taken together with the carbon atom to which they are attached, form a 3-8 membered cycloalkyl or heterocyclyl ring containing 1-2 heteroatoms selected from nitrogen, oxygen, and sulfur.

In some embodiments, R⁶ and R⁷, taken together with the carbon atom to which they are attached, form a 3-8 membered cycloalkyl. In some embodiments, R⁶ and R⁷, taken together with the carbon atom to which they are attached, form a 3-8 membered heterocyclyl ring containing 1-2 heteroatoms selected from nitrogen, oxygen, and sulfur.

In some embodiments, R⁶ and R⁷, taken together with the carbon atom to which they are attached, form a cyclopropyl, cyclobutyl, or cyclopentyl ring. In some embodiments, R⁶ and R⁷, taken together with the carbon atom to which they are attached, form an oxirane, oxetane, tetrahydrofuran, or aziridine.

In some embodiments, R⁶ and R⁷ are methyl.

In another aspect, the present invention provides an aldehyde trap compound of formulae V-a, V-b, V-c, or V-d:

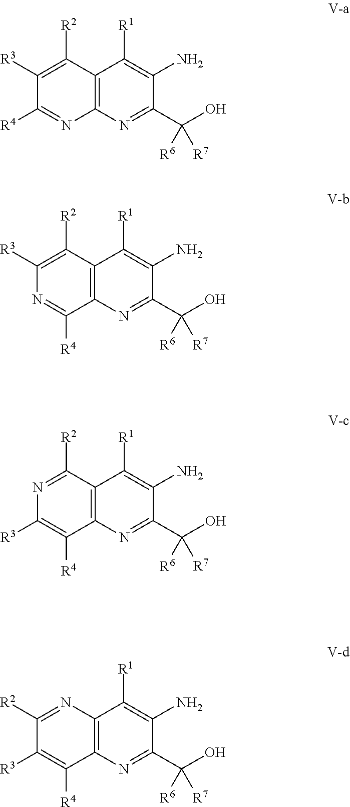

or a pharmaceutically acceptable salt thereof, wherein:
each of R, R¹, R², R³, R⁴, R⁶, and R⁷ is as defined is as defined above and described in embodiments herein, both singly and in combination.

In some embodiments, the compound is of formula V-a above.

In some embodiments, R¹ and R⁴ are H.

In some embodiments, R² is H.

In some embodiments, R⁶ and R⁷ are C₁₋₄ alkyl optionally substituted with 1, 2, or 3 deuterium or halogen atoms, or R⁶ and R⁷ are taken together with the carbon to which they are attached to form a 3-8 membered cycloalkyl ring.

In some embodiments, R³ is H, C₁₋₄ alkyl, halogen, —NR, —OR, —SR, —CO₂R, or —C(O)R, wherein R is H, optionally substituted C₁₋₄ alkyl, or optionally substituted phenyl.

In another aspect, the present invention provides an aldehyde trap compound of formulae V-e, V-f, V-g, or V-h:

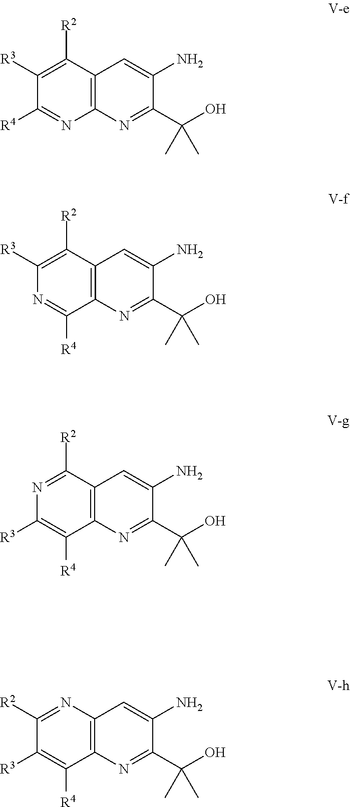

or a pharmaceutically acceptable salt thereof, wherein:
each of R, R¹, R², R³, and R⁴ is as defined is as defined above and described in embodiments herein, both singly and in combination.

In another aspect, the present invention provides an aldehyde trap compound of formulae V-i, V-j, V-k, V-l, V-m, or V-n:

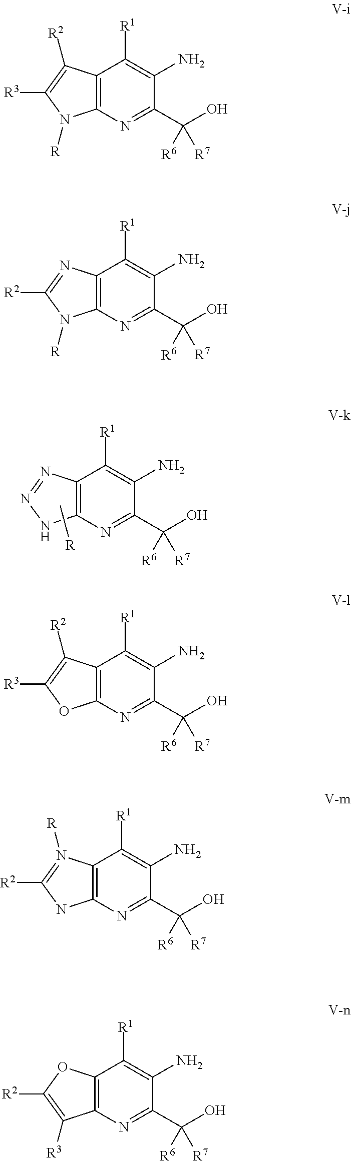

or a pharmaceutically acceptable salt thereof, wherein:
each of R, R¹, R², R³, R⁴, R⁶, and R⁷ is as defined is as defined above and described in embodiments herein, both singly and in combination.

In another aspect, the present invention provides an aldehyde trap compound of formula VI-a:

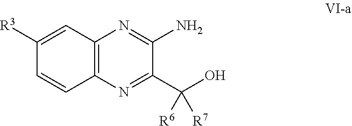

or a pharmaceutically acceptable salt thereof, wherein:
each of R, R³, R⁶, and R⁷ is as defined is as defined above and described in embodiments herein, both singly and in combination.

In some embodiments, the Scaffold of formula II is selected from those groups depicted in Table 1, below:

TABLE 1
Exemplary Scaffold Groups of Formula II

| | |
|---|---|
| 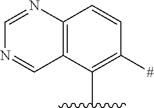 | II-1 |
| 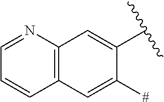 | II-2 |
| 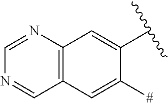 | II-3 |
| 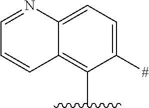 | II-4 |
| 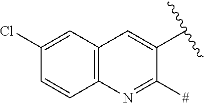 | II-5 |
| 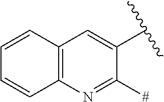 | II-6 |
| 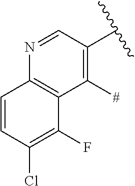 | II-7 |
| 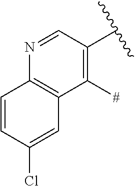 | II-8 |
| 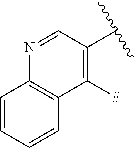 | II-9 |
| 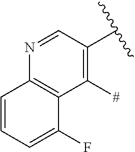 | II-10 |
| 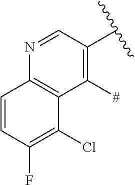 | II-11 |
| 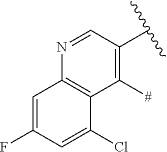 | II-12 |
| 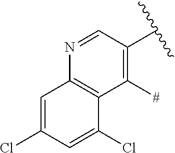 | II-13 |
| 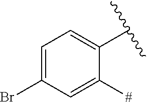 | II-14 |
| 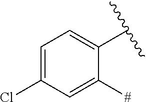 | II-15 |
| 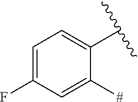 | II-16 |
| 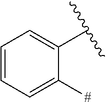 | II-17 |
| 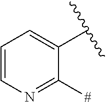 | II-18 |
| 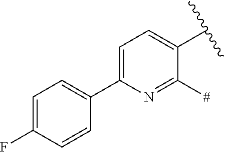 | II-19 |
| 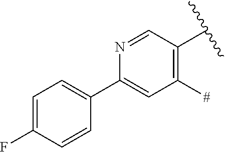 | II-20 |
| 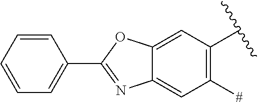 | II-21 |
| 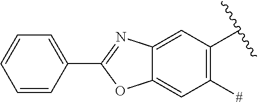 | II-22 |
| 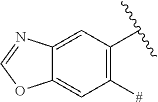 | II-23 |
| 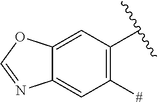 | II-24 |
| 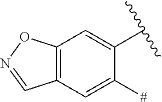 | II-25 |
| 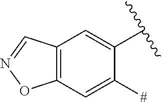 | II-26 |
| 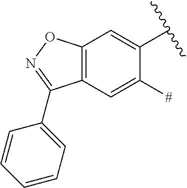 | II-27 |
| 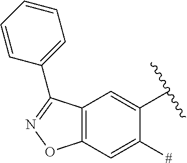 | II-28 |
| 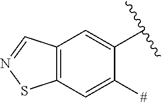 | II-29 |
| 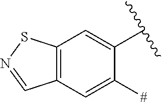 | II-30 |
| 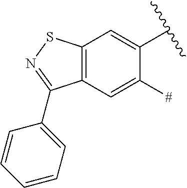 | II-31 |
| 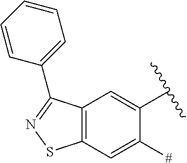 | II-32 |
| 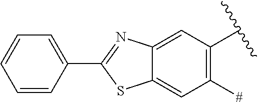 | II-33 |
| 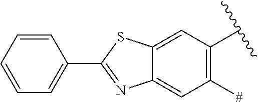 | II-34 |
| 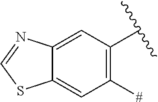 | II-35 |
| 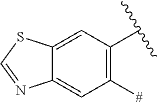 | II-36 |
| 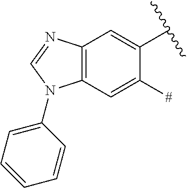 | II-37 |
| 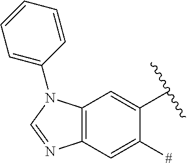 | II-38 |
| 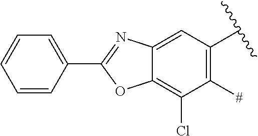 | II-39 |
| 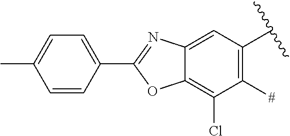 | II-40 |
| 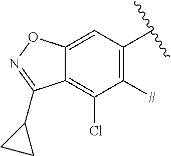 | II-41 |
| 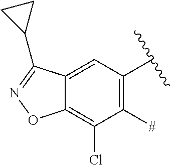 | II-42 |
| 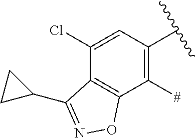 | II-43 |
| 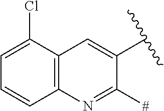 | II-44 |
| 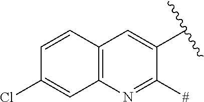 | II-45 |
| 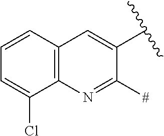 | II-46 |
| 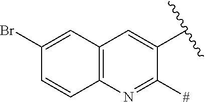 | II-47 |
| 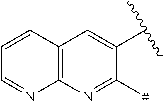 | II-48 |
| 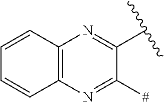 | II-49 |
| 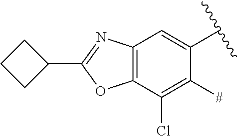 | II-50 |
| 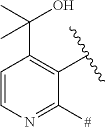 | II-51 |
| 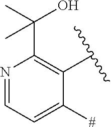 | II-52 |

wherein [symbol] is the point of attachment to the amine group and # is the point of attachment to the carbinol group.

In some embodiments, the Scaffold is selected from

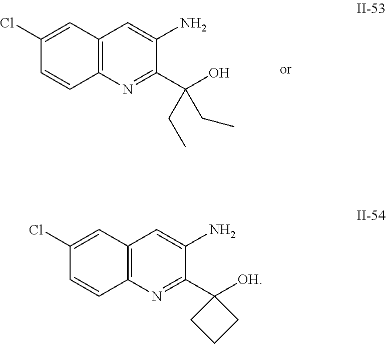

In some embodiments, Scaffold is of formula III:

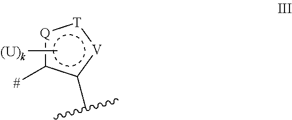

or a pharmaceutically relevant salt, wherein:
[symbol] is the point of attachment to the amine group;
# is the point of attachment to the carbinol group;
each Q, T, and V is independently selected from N or NH, S, O, CU, or CH;
[symbol] represents two double bonds within the ring, which comply with the valency requirements of the atoms and heteroatoms present in the ring;
k is 0, 1, 2, 3, or 4; and
each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
two occurances of U on adjacent carbon atoms can form an optionally substituted fused ring, selected from a fused phenyl ring; a fused 5-6 membered saturated or partially unsaturated heterocyclic ring containing 1-3 heteroatoms independently selected from nitrogen, oxygen, or sulfur; or a fused 5-6 membered heteroaryl ring containing 1-3 heteroatoms independently selected from nitrogen, oxygen, or sulfur; and
each R is independently selected from hydrogen, deuterium, or an optionally substituted group selected from C₁₋₆ aliphatic; a 3-8 membered saturated or partially unsaturated monocyclic carbocyclic ring; phenyl; an 8-10 membered bicyclic aryl ring; a 3-8 membered saturated or partially unsaturated monocyclic heterocyclic ring having 1-4 heteroatoms independently selected from nitrogen, oxygen, or sulfur; a 5-6 membered monocyclic heteroaryl ring having 1-4 heteroatoms independently selected from nitrogen, oxygen, or sulfur; a 6-10 membered bicyclic saturated or partially unsaturated heterocyclic ring having 1-5 heteroatoms independently selected from nitrogen, oxygen, or sulfur; or a 7-10 membered bicyclic heteroaryl ring having 1-5 heteroatoms independently selected from nitrogen, oxygen, or sulfur.

Each [symbol] , #, k, U, and R is as defined and described above.

As defined above and described herein, Q is selected from N or NH, S, O, CU, or CH. In some embodiments, Q is selected from N or NH, S, O, CU, or CH. In some embodiments, Q is N or NH. In some embodiments, Q is S. In some embodiments, Q is O. In some embodiments, Q is CU. In some embodiments, Q is CH.

As defined above and described herein, T is selected from N or NH, S, O, CU, or CH. In some embodiments, T is selected from N or NH, S, O, CU, or CH. In some embodiments, T is N or NH. In some embodiments, T is S. In some embodiments, T is O. In some embodiments, T is CU. In some embodiments, T is CH.

As defined above and described herein, V is selected from N or NH, S, O, CU, or CH. In some embodiments, V is selected from N or NH, S, O, CU, or CH. In some embodiments, V is N or NH. In some embodiments, V is S. In some embodiments, V is O. In some embodiments, V is CU. In some embodiments, V is CH.

As defined above and described herein, k is 0, 1, 2, 3, or 4. In some embodiments k is O. In some embodiments, k is 1. In some embodiments, k is 2. In some embodiments, k is 3. In some embodiments, k is 4.

As defined above and described herein, [symbol] represents two double bonds within the ring, which comply with the valency requirements of the atoms and heteroatoms present in the ring. In some embodiments, the ring formed is thiophene. In some embodiments, the ring formed is oxazole. In some embodiments, the ring formed is isothiazole.

In some embodiments, one or more of Q and V are CH; T is S; [symbol] is arranged to form a thiophene; and k is O. In some embodiments, one or more of Q is CH; T is N or NH; V is O; [symbol] is arranged to form an isoxazole; and k is O. In some embodiments, one or more of Q is S; T and V are CH; [symbol] is arranged to form a thiophene; k is 1; and U is —S(O)₂R. In some embodiments, one or more of Q is S; T and V are CH; [symbol] is arranged to form a thiophene; k is 1; and U is —S(O)₂CH₃. In some embodiments, one or more of Q is CH; T is N or NH; V is S; [symbol] is arranged to form an isothiazole; and k is 0.

In some embodiments, the Scaffold of formula III is selected from those groups depicted in Table 2, below:

TABLE 2
Exemplary Scaffold Groups of Formula III

| | |
|---|---|
| 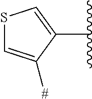 | III-1 |
| 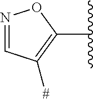 | III-2 |
| 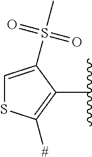 | III-3 |
| 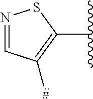 | III-4 |

wherein [symbol] is the point of attachment to the amine group and # is the point of attachment to the carbinol group.

In some embodiments, Scaffold is of formulae IV-A or IV-B:

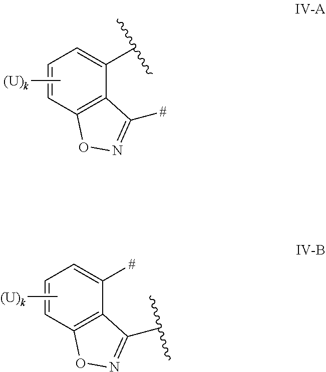

or a pharmaceutically acceptable salt thereof, wherein:
[symbol] is the point of attachment to the amine moiety;
# is the point of attachment to the carbinol moiety;
k is 0, 1, 2, 3, or 4; and
each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
two occurances of U on adjacent carbon atoms can form an optionally substituted fused ring, selected from a fused phenyl ring; a fused 5-6 membered saturated or partially unsaturated heterocyclic ring containing 1-3 heteroatoms independently selected from nitrogen, oxygen, or sulfur; or a fused 5-6 membered heteroaryl ring containing 1-3 heteroatoms independently selected from nitrogen, oxygen, or sulfur; and
each R is independently selected from hydrogen, deuterium, or an optionally substituted group selected from C₁₋₆ aliphatic; a 3-8 membered saturated or partially unsaturated monocyclic carbocyclic ring; phenyl; an 8-10 membered bicyclic aryl ring; a 3-8 membered saturated or partially unsaturated monocyclic heterocyclic ring having 1-4 heteroatoms independently selected from nitrogen, oxygen, or sulfur; a 5-6 membered monocyclic heteroaryl ring having 1-4 heteroatoms independently selected from nitrogen, oxygen, or sulfur; a 6-10 membered bicyclic saturated or partially unsaturated heterocyclic ring having 1-5 heteroatoms independently selected from nitrogen, oxygen, or sulfur; or a 7-10 membered bicyclic heteroaryl ring having 1-5 heteroatoms independently selected from nitrogen, oxygen, or sulfur.

Each of [symbol] , #, k, U, and R is as defined and described above.

In some embodiments, the Scaffold of formulae IV-A or IV-B is selected from those groups depicted in Table 3, below:

TABLE 3
Exemplary Scaffold Groups of Formula IV

| | |
|---|---|
| 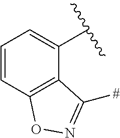 | IV-1 |
| 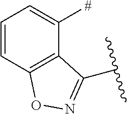 | IV-2 |

wherein [symbol] is the point of attachment to the amine group and # is the point of attachment to the carbinol group.

As defined above and described herein, the method requires the step of contacting the compound of formula A with a biologically relevant aldehyde to form a conjugate of formula I

In some embodiments, the biologically relevant aldehyde is selected from formaldehyde, acetaldehyde, acrolein, glyoxal, methylglyoxal, hexadecanal, octadecanal, hexadecenal, succinic semi-aldehyde, malondialdehyde, 4-hydroxynonenal, 4-hydroxy-2E-hexenal, 4-hydroxy-2E,6Z-dodecadienal, retinaldehyde, leukotriene B4 aldehyde, and octadecenal.

In some embodiments, the biologically relevant aldehyde is formaldehyde. In some embodiments, the biologically relevant aldehyde is acetaldehyde. In some embodiments, the biologically relevant aldehyde is acrolein. In some embodiments, the biologically relevant aldehyde is glyoxal. In some embodiments, the biologically relevant aldehyde is methylglyoxal. In some embodiments, the biologically relevant aldehyde is hexadecanal. In some embodiments, the biologically relevant aldehyde is octadecanal. In some embodiments, the biologically relevant aldehyde is hexadecenal. In some embodiments, the biologically relevant aldehyde is succinic semi-aldehyde (SSA). In some embodiments, the biologically relevant aldehyde is malondialdehyde (MDA). In some embodiments, the biologically relevant aldehyde is 4-hydroxynonenal. In some embodiments, the biologically relevant aldehyde is retinaldehyde. In some embodiments, the biologically relevant aldehyde is 4-hydroxy-2E-hexenal. In some embodiments, the biologically relevant aldehyde is 4-hydroxy-2E,6Z-dodecadienal. In some embodiments, the aldehyde is leukotriene B4 aldehyde. In some embodiments, the aldehyde is octadecenal.

In some embodiments, the biologically relevant aldehyde is selected from those compounds depicted in Table 4, below:

TABLE 4
Exemplary Biologically Relevant Aldehydes

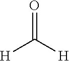

formaldehyde

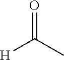

acetaldehyde

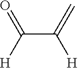

acrolein

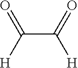

glyoxal

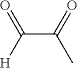

methylglyoxal

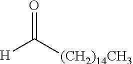

hexadecanal

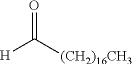

octadecanal

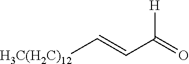

hexadecenal

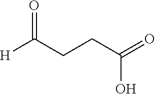

succinic semi-aldehyde

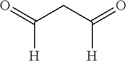

malondialdehyde

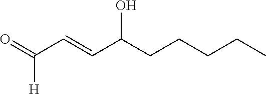

4-hydroxynonenal (HNE)

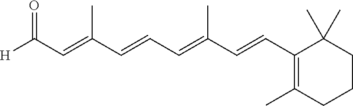 [SMILES: CC1=C(/C=C/C(C)=C/C=C/C(C)=C/C=O)C(C)(C)CCC1]

retinaldehyde

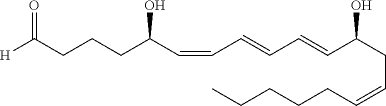 [SMILES: CCCCC/C=C\C[C@H](O)/C=C/C=C/C=C\[C@H](O)CCCC=O]

leukotriene B4 aldehyde

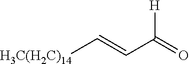

octadecenal

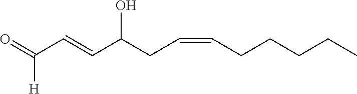

4-HDDE

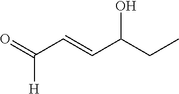

4-HHE

In some embodiments, the compound of formula A is

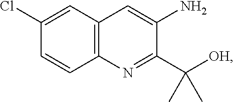 [SMILES: CC(C)(O)c1nc2ccc(Cl)cc2cc1N]

and the biologically relevant aldehyde is selected from formaldehyde, acetaldehyde, acrolein, glyoxal, methylglyoxal, hexadecanal, octadecanal, hexadecenal, succinic semi-aldehyde, malondialdehyde, 4-hydroxynonenal, 4-hydroxy-2E-hexenal, 4-hydroxy-2E,6Z-dodecadienal, retinaldehyde, leukotriene B4 aldehyde, and octadecenal. In some embodiments, the compound of formula A is

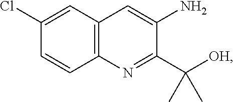 [SMILES: CC(C)(O)c1nc2ccc(Cl)cc2cc1N]

and the biologically relevant aldehyde is selected from those within Table 4. In some embodiments, the compound of formula A is

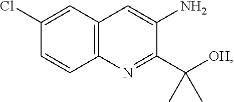 [SMILES: CC(C)(O)c1nc2ccc(Cl)cc2cc1N]

and the biologically relevant aldehyde is succinic semi-aldehyde.

In some embodiments, a provided method results in the formation of a compound of formula I:

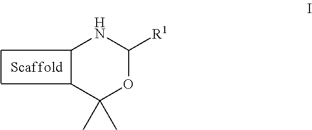

wherein:
Scaffold is as defined above and described herein; and
R¹ is selected from the side-chain of a biologically relevant aldehyde as defined above and described herein.

As defined above and described herein, R¹ is selected from the side-chain of a biologically relevant aldehyde as defined above. As defined above and described herein, R¹ is selected from those groups, below:

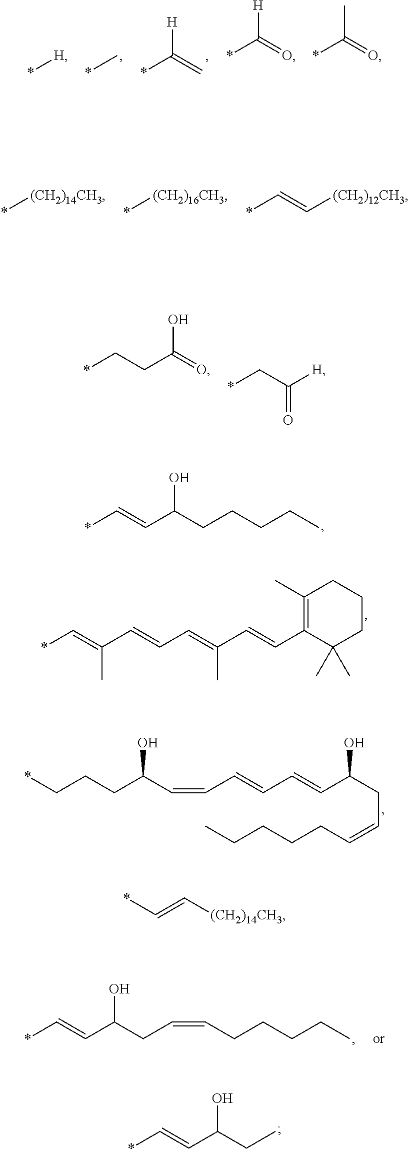

wherein * indicates the point of attachment of IV to the rest of the molecule.

In some embodiments, a provided method results in formation of a conjugate of formula I selected from those compounds depicted in Table 5, below:

TABLE 5
Exemplary Conjugates of Formula I

| | |
|---|---|
| 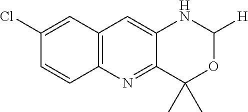 [SMILES: CC1(C)OCNc2cc3cc(Cl)ccc3nc21] | I-1 |
| 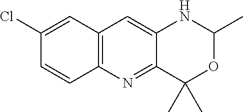 [SMILES: CC1Nc2cc3cc(Cl)ccc3nc2C(C)(C)O1] | I-2 |
| 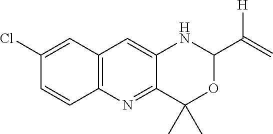 [SMILES: C=CC1Nc2cc3cc(Cl)ccc3nc2C(C)(C)O1] | I-3 |
| 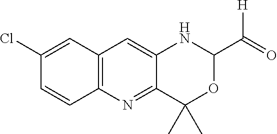 [SMILES: CC1(C)OC(C=O)Nc2cc3cc(Cl)ccc3nc21] | I-4 |
| 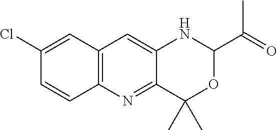 [SMILES: CC(=O)C1Nc2cc3cc(Cl)ccc3nc2C(C)(C)O1] | I-5 |
| 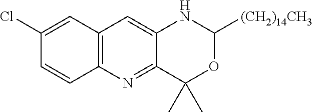 | I-6 |
| 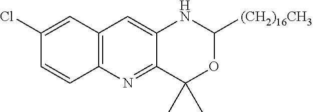 | I-7 |
| 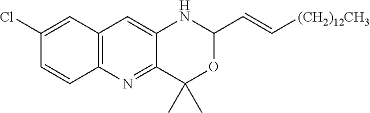 | I-8 |
| 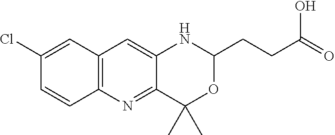 [SMILES: CC1(C)OC(CCC(=O)O)Nc2cc3cc(Cl)ccc3nc21] | I-9 |
| 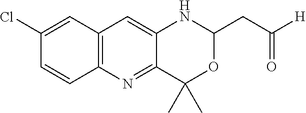 [SMILES: CC1(C)OC(CC=O)Nc2cc3cc(Cl)ccc3nc21] | I-10 |
| 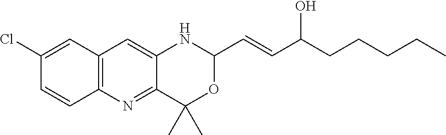 [SMILES: CCCCCC(O)/C=C/C1Nc2cc3cc(Cl)ccc3nc2C(C)(C)O1] | I-11 |
| 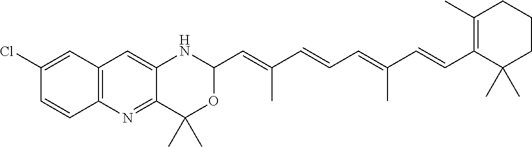 [SMILES: CC1=C(/C=C/C(C)=C/C=C/C(C)=C/C2Nc3cc4cc(Cl)ccc4nc3C(C)(C)O2)C(C)(C)CCC1] | I-12 |
| 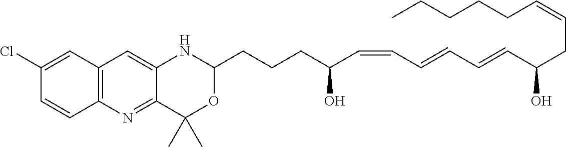 [SMILES: CCCCC/C=C\C[C@@H](O)/C=C/C=C/C=C\[C@@H](O)CCCC1Nc2cc3cc(Cl)ccc3nc2C(C)(C)O1] | I-13 |
| 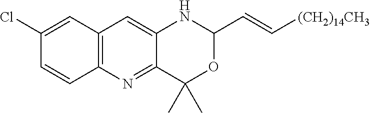 | I-14 |
| 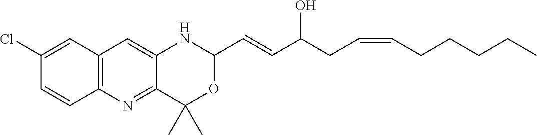 [SMILES: CCCCC/C=C\CC(O)/C=C/C1Nc2cc3cc(Cl)ccc3nc2C(C)(C)O1] | I-15 |
| 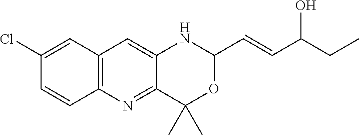 [SMILES: CCC(O)/C=C/C1Nc2cc3cc(Cl)ccc3nc2C(C)(C)O1] | I-16 |

In some embodiments, the present invention provides a conjugate of formula I:

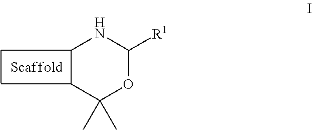

wherein:
Scaffold is a moiety to which the amino group and the carbinol group are attached such that the resulting amino-carbinol moiety is capable of trapping an aldehyde moiety; and
R¹ is the side-chain of a biologically relevant aldehyde.

Each of Scaffold and R¹ is as defined and described above.

In some embodiments, present invention provides a method of treating a patient in need thereof, comprising
(a) administering a compound of formula A:

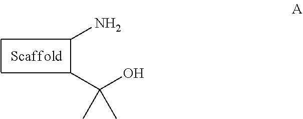

or a pharmaceutically acceptable salt thereof, wherein:
Scaffold is a moiety to which the amino group and the carbinol group are attached such that the resulting amino-carbinol moiety is capable of trapping an aldehyde moiety; and
(b) contacting said compound of formula A with a biologically relevant aldehyde to form a conjugate of formula I:

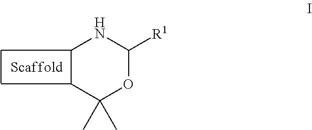

wherein:
R¹ is the side-chain of a biologically relevant aldehyde.

Each of Scaffold, compound of formula A, biologically relevant aldehyde, conjugate of formula I, R¹, or any combination thereof is as defined and described herein.

In some embodiments, present invention provides a method of:
(a) contacting a compound of formula A:

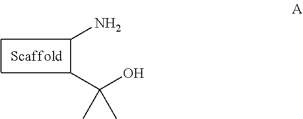

or a pharmaceutically acceptable salt thereof, wherein:
Scaffold is a moiety to which the amino group and the carbinol group are attached such that the resulting amino-carbinol moiety is capable of trapping an aldehyde moiety; and
(b) contacting said compound of formula A with a biologically relevant aldehyde in situ to form a conjugate of formula I:

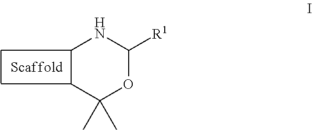

wherein:
R¹ is the side-chain of a biologically relevant aldehyde.

Each of scaffold, compound of formula A, biologically relevant aldehyde, conjugate of formula I, R¹, or any combination thereof is as defined and described herein.

In some embodiments, present invention provides a method of:
(a) contacting a compound of formula A:

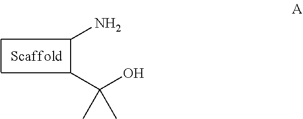

or a pharmaceutically acceptable salt thereof, wherein:
Scaffold is a moiety to which the amino group and the carbinol group are attached such that the resulting amino-carbinol moiety is capable of trapping an aldehyde moiety; and
(b) contacting said compound of formula A with a biologically relevant aldehyde in vivo to form a conjugate of formula I:

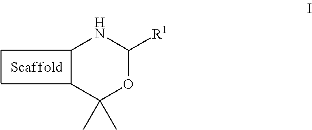

wherein:
R¹ is the side-chain of a biologically relevant aldehyde.

Each of scaffold, compound of formula A, biologically relevant aldehyde, conjugate of formula I, R¹, or any combination thereof is as defined and described herein.

### 4. Uses of Compounds and Pharmaceutically Acceptable Compositions Thereof

In certain embodiments, the present invention provides compounds, compositions, and methods for treatment, prevention, and/or reduction of a risk of diseases, disorders, or conditions in which aldehyde toxicity is implicated in the pathogenesis. In some embodiments, such compounds include those of the formulae described herein, or a pharmaceutically acceptable salt thereof, wherein each variable is as defined and described herein. According to one aspect, the present invention provides a method of contacting a biologically relevant aldehyde with an amino-carbinol-containing compound to form a conjugate of formula I.

Certain compounds described herein are found to be useful in scavenging toxic aldehydes, such as MDA and HNE. The compounds described herein undergo a Schiff base condensation with MDA, HNE, or other toxic aldehydes, and form a complex with the aldehydes in an energetically favorable reaction, thus reducing or eliminating aldehydes available for reaction with a protein, lipid, carbohydrate, or DNA. Importantly, compounds described herein can react with aldehydes to form a compound having a closed-ring structure that contains the aldehydes, thus trapping the aldehydes and preventing the aldehydes from being released back into the cellular milieu.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent, delay or lessen the severity of their recurrence.

The invention relates to compounds described herein for the treatment, prevention, and/or reduction of a risk of diseases, disorders, or conditions in which aldehyde toxicity is implicated in the pathogenesis.

Examples of the diseases, disorders, or conditions in which aldehyde toxicity is implicated include an ocular disease, disorder, or condition, including, but not limited to, a corneal disease (e.g., dry eye syndrome, cataracts, keratoconus, bullous and other keratopathy, and Fuch's endothelial dystrophy), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions associated with high aldehyde levels as a result of inflammation (e.g., uveitis, scleritis, ocular Stevens Johnson Syndrome, ocular rosacea (with or without meibomian gland dysfunction)). In one example, the ocular disease, disorder, or condition is not macular degeneration, such as age-related macular degeneration (“AMD”), or Stargardt's disease. In a further example, the ocular disease, disorder, or condition is dry eye syndrome, ocular rosacea, or uveitis.

Examples of the diseases, disorders, conditions, or indications in which aldehyde toxicity is implicated also include non-ocular disorders, including psoriasis, topical (discoid) lupus, contact dermatitis, atopic dermatitis, allergic dermatitis, radiation dermatitis, acne vulgaris, Sjogren-Larsson Syndrome and other ichthyosis, solar elastosis/wrinkles, skin tone firmness, puffiness, eczema, smoke or irritant induced skin changes, dermal incision, a skin condition associated burn and/or wound, lupus, scleroderma, asthma, chronic obstructive pulmonary disease (COPD), rheumatoid arthritis, inflammatory bowel disease, sepsis, atherosclerosis, ischemic-reperfusion injury, Parkinson's disease, Alzheimer's disease, succinic semialdehyde dehydrogenase deficiency, multiple sclerosis, amyotrophic lateral sclerosis, diabetes, metabolic syndrome, age-related disorders, and fibrotic diseases. In a further example, the non-ocular disorder is a skin disease, disorder, or condition selected from contact dermatitis, atopic dermatitis, allergic dermatitis, and. radiation dermatitis. In another example, the non-ocular disorder is a skin disease, disorder, or condition selected from Sjogren-Larsson Syndrome and a cosmetic indication associated burn and/or wound.

In a further example, the diseases, disorders, or conditions in which aldehyde toxicity is implicated are an age-related disorder. Examples of age-related diseases, disorders, or conditions include wrinkles, dryness, and pigmentation of the skin.

Examples of the diseases, disorders, or conditions in which aldehyde toxicity is implicated further include conditions associated with the toxic effects of blister agents or burns from alkali agents. The compounds described herein reduce or eliminate toxic aldehydes and thus treat, prevent, and/or reduce a risk of these diseases or disorders.

In one embodiment, the invention relates to the treatment, prevention, and/or reduction of a risk of an ocular disease, disorder, or condition in which aldehyde toxicity is implicated in the pathogenesis, comprising administering to a subject in need thereof a compound described herein. The ocular disease, disorder, or condition includes, but is not limited to, a corneal disease (e.g., dry eye syndrome, cataracts, keratoconus, bullous and other keratopathy, and Fuch's endothelial dystrophy in the cornea), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions where inflammation leads to high aldehyde levels (e.g., uveitis, scleritis, ocular Stevens Johnson Syndrome, ocular rosacea (with or without meibomian gland dysfunction)). The ocular disease, disorder, or condition does not include macular degeneration, such as AMD, or Stargardt's disease. In one illustration, in the ocular disease, disorder, or condition, the amount or concentration of MDA or HNE is increased in the ocular tissues or cells. For example, the amount or concentration of aldehydes (e.g., MDA or HNE) is increased for at least 1.1 fold, 1.2 fold, 1.3 fold, 1.4 fold, 1.5 fold, 2 fold, 2.5 fold, 5 fold, 10 fold as compared to that in normal ocular tissues or cells. Compounds described herein decrease aldehyde (e.g., MDA and HNE) concentration in a time-dependent manner. The amount or concentration of aldehydes (e.g., MDA or FINE) can be measured by methods or techniques known in the art, such as those described in Tukozkan et al., 2006, Furat Tip Dergisi 11: 88-92.

In one class, the ocular disease, disorder, or condition is dry eye syndrome. In a second class, the ocular disease, disorder, or condition is a condition associated with PRK healing and other corneal healing. For example, the invention is directed to advancing PRK healing or other corneal healing, comprising administering to a subject in need thereof a compound described herein. In a third class, the ocular disease, disorder, or condition is an ocular condition associated with high aldehyde levels as a result of inflammation (e.g., uveitis, scleritis, ocular Stevens Johnson Syndrome, and ocular rosacea (with or without meibomian gland dysfunction). In a fourth class, the ocular disease, disorder, or condition is keratoconus, cataracts, bullous and other keratopathy, Fuchs' endothelial dystrophy, ocular cicatricial pemphigoid, or allergic conjunctivitis. The compound described herein may be administered topically or systemically, as described herein below.

In a second embodiment, the invention relates to the treatment, prevention, and/or reduction of a risk of a skin disorder or condition or a cosmetic indication, in which aldehyde toxicity is implicated in the pathogenesis, comprising administering to a subject in need thereof a compound described herein. The skin disorder or condition includes, but is not limited to, psoriasis, scleroderma, topical (discoid) lupus, contact dermatitis, atopic dermatitis, allergic dermatitis, radiation dermatitis, acne vulgaris, and Sjogren-Larsson Syndrome and other ichthyosis, and the cosmetic indication is solar elastosis/wrinkles, skin tone firmness, puffiness, eczema, smoke or irritant induced skin changes, dermal incision, or a skin condition associated burn and/or wound. In some embodiments, the invention related to age-related diseases, disorders, or conditions of the skin, as described herein.

Various skin disorders or conditions, such as atopic dermatitis, topical (discoid) lupus, psoriasis and scleroderma, are characterized by high MDA and HNE levels (Niwa et al., 2003, Br J Dermatol. 149:248; Sikar Aktürk et al., 2012, J Eur Acad Dermatol Venereol. 26: 833; Tikly et al, 2006, Clin Rheumatol. 25(3):320-4). In addition, ichthyosis characteristic of the Sjogren-Larsson Syndrome (SLS) originates from accumulation of fatty aldehydes, which disrupts the normal function and secretion of lamellar bodies (LB) and leads to intercellular lipid deposits in the Strateum Corneum (SC) and a defective water barrier in the skin layer (Rizzo et al., 2010, Arch Dermatol Res. 302(6):443-51). The enzyme fatty aldehyde dehydrogenase that metabolizes aldehydes is dysfunctional in SLS patients. Thus, compounds that reduce or eliminate aldehydes, such as the compounds described herein, can be used to treat, prevent, and/or reduction of a risk of skin disorders or conditions in which aldehyde toxicity is implicated in the pathogenesis, such as those described herein. Furthermore, with an improvement to the water barrier and prevention of aldehyde-mediated inflammation (including fibrosis and elastosis (Chairpotto et al. (2005)), many cosmetic indications, such as solar elastosis/wrinkles, skin tone, firmness (puffiness), eczema, smoke or irritant induced skin changes and dermal incision cosmesis, and skin conditions associated with burn and/or wound can be treated using the method of the invention.

In one class, the skin disease, disorder, or condition is psoriasis, scleroderma, topical (discoid) lupus, contact dermatitis, atopic dermatitis, allergic dermatitis, radiation dermatitis, acne vulgaris, or Sjogren-Larsson Syndrome and other ichthyosis. In one exemplification, the skin disease, disorder, or condition is contact dermatitis, atopic dermatitis, allergic dermatitis, radiation dermatitis, or Sjogren-Larsson Syndrome and other ichthyosis. In a second class, the cosmetic indication is solar elastosis/wrinkles, skin tone firmness, puffiness, eczema, smoke or irritant induced skin changes, dermal incision, or a skin condition associated burn and/or wound.

In a third embodiment, the invention relates to the treatment, prevention, and/or reduction of a risk of a condition associated with the toxic effects of blister agents or burns from alkali agents in which aldehyde toxicity is implicated in the pathogenesis, comprising administering to a subject in need thereof a compound described herein.

Blister agents include, but are not limited to, sulfur mustard, nitrogen mustard, and phosgene oxime. Toxic or injurious effects of blister agents include pain, irritation, and/or tearing in the skin, eye, and/or mucous, and conjunctivitis and/or corneal damage to the eye. Sulfur mustard is the compound bis(2-chlorethyl) sulfide. Nitrogen mustard includes the compounds bis(2-chlorethyl)ethylamine, bis(2-chlorethyl)methylamine, and tris(2-chlorethyl)amine. Sulfur mustard or its analogs can cause an increase in oxidative stress and in particular in HNE levels, and by depleting the antioxidant defense system and thereby increasing lipid peroxidation, may induce an oxidative stress response and thus increase aldehyde levels (Jafari et al., 2010, Clin Toxicol (Phila). 48(3):184-92; Pal et al., 2009, Free Radic Biol Med. 47(11):1640-51). Antioxidants, such as Silibinin, when applied topically, attenuate skin injury induced from exposure to sulfur mustard or its analogs, and increased activities of antioxidant enzymes may be a compensatory response to reactive oxygen species generated by the sulfur mustard (Jafari et al. supra; Tewari-Singh et al., 2012, PLoS One 7(9):e46149). Further, intervention to reduce free radical species was an effective treatment post exposure for phosgene induced lung injury (Sciuto et al. (2004)). Thus, compounds that reduce or eliminate aldehydes, such as compounds described herein, can be used to treat, prevent, and/or reduce a risk of a condition associated with the toxic effects of blister agents, such as sulfur mustard, nitrogen mustard, and phosgene oxime.

Alkali agents include, but are not limited to, lime, lye, ammonia, and drain cleaners. Compounds that reduce or eliminate aldehydes, such as compounds described herein, can be used to treat, prevent, and/or reduce a risk of a condition associated with burns from an alkali agent.

In a fourth embodiment, the invention relates to the treatment, prevention, and/or reduction of a risk of an autoimmune, immune-mediated, inflammatory, cardiovascular, or neurological disease, disorder, or condition, or metabolic syndrome, or diabetes, in which aldehyde toxicity is implicated in the pathogenesis, comprising administering to a subject in need thereof a compound described herein. The autoimmune or immune-mediated disease, disorder, or condition includes, but is not limited to, lupus, scleroderma, asthma, chronic obstructive pulmonary disease (COPD), and rheumatoid arthritis. The inflammatory disease, disorder, or condition includes, but is not limited to, rheumatoid arthritis, inflammatory bowel disease (e.g., Crohn's disease and ulcerative colitis), sepsis, and fibrosis (e.g., renal, hepatic, pulmonary, and cardiac fibrosis). The cardiovascular disease, disorder, or condition includes, but is not limited to, atherosclerosis and ischemic-reperfusion injury. The neurological disease, disorder, or condition includes, but is not limited to, Parkinson's disease, Alzheimer's disease, succinic semialdehyde dehydrogenase deficiency, multiple sclerosis, amyotrophic lateral sclerosis, and the neurological aspects of Sjogren-Larsson Syndrome (cognitive delay and spasticity).

A skilled person would understand that the disease, disorder, or condition listed herein may involve more than one pathological mechanism. For example, a disease, disorder, or condition listed herein may involve dysregulation in the immunological response and inflammatory response. Thus, the above categorization of a disease, disorder, or condition is not absolute, and the disease, disorder, or condition may be considered an immunological, an inflammatory, a cardiovascular, a neurological, and/or metabolic disease, disorder, or condition.

Individuals with deficiencies in aldehyde dehydrogenase are found to have high aldehyde levels and increased risk of Parkinson's disease (Fitzmaurice et al., 2013, Proc Natl Acad Sci USA. 110(2):636-41) and Alzheimer's disease (Kamino et al., 2000, Biochem Biophys Res Commun. 273:192-6). In Parkinson's disease, aldehydes specifically interfere with dopamine physiology (Reed, 2011, Free Radic Biol Med. 51:1302-19; Zarkovic et al., 2003, Mol Aspects Med. 24: 293-303; Wood et al., 2007, Brain Res. 1145: 150-6). In addition, aldehydes levels are elevated in multiple sclerosis, amyotrophic lateral sclerosis, autoimmune diseases such as lupus, rheumatoid arthritis, lupus, psoriasis, scleroderma, and fibrotic diseases, and increased levels of HNE and MDA are implicated in the progression of atherosclerosis and diabetes (Aldini et al., 2011, J Cell Mol Med. 15:1339-54; Wang et al., 2010, Arthritis Rheum. 62: 2064-72; Amara et al., Clin Exp Immunol. 101:233-8 (1995); Hassan et al., 2011, Int J Rheum Dis. 14: 325-31; Sikar et al., 2012, J Eur Acad Dermatol Venereol. 26(7):833-7; Tikly et al., 2006, Clin Rheumatol. 25:320-4; Albano et al., 2005, Gut 54:987-93; Pozzi et al., 2009, J Am Soc Nephrol 20:2119-25). MDA is further implicated in the increased formation of foam cells leading to atherosclerosis (Leibundgut et al., 2013, Curr Opin Pharmacol. 13:168-279). Also, aldehyde-related toxicity plays an important role in the pathogenesis of many inflammatory lung diseases, such as asthma and chronic obstructive pulmonary disease (COPD) (Bartoli et al., 2011, Mediators of Inflammation 2011, Article 891752). Thus, compounds that reduce or eliminate aldehydes, such as compounds described herein, can be used to treat, prevent, and/or reduce a risk of an autoimmune, immune-mediated, inflammatory, cardiovascular, or neurological disease, disorder, or condition, or metabolic syndrome, or diabetes. For example, compounds described herein, such as II-5, prevent aldehyde-mediated cell death in neurons. Further, compounds described herein, such as II-5, downregulate a broad spectrum of pro-inflammatory cytokines and/or upregulate anti-inflammatory cytokines, which indicates that compounds described herein are useful in treating inflammatory diseases, such as multiple sclerosis and amyotrophic lateral sclerosis.

As discussed above, a disclosed composition may be administered to a subject in order to treat or prevent macular degeneration and other forms of retinal disease whose etiology involves the accumulation of A2E and/or lipofuscin. Other diseases, disorders, or conditions characterized by the accumulation A2E may be similarly treated.

In one embodiment, a compound is administered to a subject that reduces the formation of A2E. For example, the compound may compete with PE for reaction with trans-RAL, thereby reducing the amount of A2E formed. In another embodiment, a compound is administered to a subject that prevents the accumulation of A2E. For example, the compound competes so successfully with PE for reaction with trans-RAL, no A2E is formed.

Individuals to be treated fall into three groups: (1) those who are clinically diagnosed with macular degeneration or other forms of retinal disease whose etiology involves the accumulation of A2E and/or lipofuscin on the basis of visual deficits (including but not limited to dark adaptation, contrast sensitivity and acuity) as determined by visual examination and/or electroretinography, and/or retinal health as indicated by fundoscopic examination of retinal and RPE tissue for drusen accumulations, tissue atrophy and/or lipofuscin fluorescence; (2) those who are pre-symptomatic for macular degenerative disease but thought to be at risk based on abnormal results in any or all of the same measures; and (3) those who are pre-symptomatic but thought to be at risk genetically based on family history of macular degenerative disease and/or genotyping results showing one or more alleles or polymorphisms associated with the disease. The compositions are administered topically or systemically at one or more times per month, week or day. Dosages may be selected to avoid side effects, if any, on visual performance in dark adaptation. Treatment is continued for a period of at least one, three, six, or twelve or more months. Patients may be tested at one, three, six, or twelve months or longer intervals to assess safety and efficacy. Efficacy is measured by examination of visual performance and retinal health as described above.

In one embodiment, a subject is diagnosed as having symptoms of macular degeneration, and then a disclosed compound is administered. In another embodiment, a subject may be identified as being at risk for developing macular degeneration (risk factors include a history of smoking, age, female gender, and family history), and then a disclosed compound is administered. In another embodiment, a subject may have dry AMD in both eye, and then a disclosed compound is administered. In another embodiment, a subject may have wet AMD in one eye but dry AMD in the other eye, and then a disclosed compound is administered. In yet another embodiment, a subject may be diagnosed as having Stargardt disease and then a disclosed compound is administered. In another embodiment, a subject is diagnosed as having symptoms of other forms of retinal disease whose etiology involves the accumulation of A2E and/or lipofuscin, and then the compound is administered. In another embodiment a subject may be identified as being at risk for developing other forms of retinal disease whose etiology involves the accumulation of A2E and/or lipofuscin, and then the disclosed compound is administered. In some embodiments, a compound is administered prophylactically. In some embodiments, a subject has been diagnosed as having the disease before retinal damage is apparent. For example, a subject is found to carry a gene mutation for ABCA4 and is diagnosed as being at risk for Stargardt disease before any ophthalmologic signs are manifest, or a subject is found to have early macular changes indicative of macular degeneration before the subject is aware of any effect on vision. In some embodiments, a human subject may know that he or she is in need of the macular generation treatment or prevention.

In some embodiments, a subject may be monitored for the extent of macular degeneration. A subject may be monitored in a variety of ways, such as by eye examination, dilated eye examination, fundoscopic examination, visual acuity test, and/or biopsy. Monitoring can be performed at a variety of times. For example, a subject may be monitored after a compound is administered. The monitoring can occur, for example, one day, one week, two weeks, one month, two months, six months, one year, two years, five years, or any other time period after the first administration of a compound. A subject can be repeatedly monitored. In some embodiments, the dose of a compound may be altered in response to monitoring.

In some embodiments, the disclosed methods may be combined with other methods for treating or preventing macular degeneration or other forms of retinal disease whose etiology involves the accumulation of A2E and/or lipofuscin, such as photodynamic therapy. For example, a patient may be treated with more than one therapy for one or more diseases or disorders. For example, a patient may have one eye afflicted with dry form AMD, which is treated with a compound of the invention, and the other eye afflicted with wet form AMD which is treated with, e.g., photodynamic therapy.

In some embodiments, a compound for treating or preventing macular degeneration or other forms of retinal disease whose etiology involves the accumulation of A2E and/or lipofuscin may be administered chronically. The compound may be administered daily, more than once daily, twice a week, three times a week, weekly, biweekly, monthly, bimonthly, semiannually, annually, and/or biannually.

Sphingosine 1-phosphate, a bioactive signaling molecule with diverse cellular functions, is irreversibly degraded by the endoplasmic reticulum enzyme sphingosine 1-phosphate lyase, generating trans-2-hexadecenal and phosphoethanolamine. It has been demonstrated that trans-2-hexadecenal causes cytoskeletal reorganization, detachment, and apoptosis in multiple cell types via a JNK-dependent pathway. See Upadhyaya et al., 2012, Biochem Biophys Res Commun. 424(1):18-21. These findings and the known chemistry of related α,β-unsaturated aldehydes raise the possibility that trans-2-hexadecenal interact with additional cellular components. It was shown that it reacts readily with deoxyguanosine and DNA to produce the diastereomeric cyclic 1,N(2)-deoxyguanosine adducts 3-(2-deoxy-β-d-erythro-pentofuranosyl)-5,6,7,8-tetrahydro-8R-hydroxy-6R-tridecylpyrimido[1,2-a]purine-10(3H)one and 3-(2-deoxy-β-d-erythro-pentofuranosyl)-5,6,7,8-tetrahydro-8S-hydroxy-6S-tridecylpyrimido[1,2-a]purine-10(3H)one. These findings demonstrate that trans-2-hexadecenal produced endogenously by sphingosine 1-phosphate lyase react directly with DNA forming aldehyde-derived DNA adducts with potentially mutagenic consequences.

Succinic semialdehyde dehydrogenase deficiency (SSADHD), also known as 4-hydroxybutyric aciduria or gamma-hydroxybutyric aciduria, is the most prevalent autosomal-recessively inherited disorder of GABA metabolism (Vogel et al, 2013, J Inherit Metab Dis. 36(3):401-10), manifests a phenotype of developmental delay and hypotonia in early childhood, and severe expressive language impairment and obsessive-compulsive disorder in adolescence and adulthood. Epilepsy occurs in half of patients, usually as generalized tonic-clonic seizures although sometimes absence and myoclonic seizures occur (Pearl et al., 2014, Dev Med Child Neurol., doi: 10.1111/dmcn.12668.). Greater than two-thirds of patients manifest neuropsychiatric problems (i.e., ADHD, OCD and aggression) in adolescence and adulthood, which can be disabling. Metabolically, there is accumulation of the major inhibitory neurotransmitter GABA and gamma-hydroxybutyrate (GHB), a neuromodulatory monocarboxylic acid (Snead and Gibson, 2005, N Engl J Med. 352(26):2721-32). In addition, several other intermediates specific to this disorder have been detected both in patients and the corresponding murine model. Vigabatrin (VGB; gamma-vinylGABA), an irreversible inhibitor of GABA-transaminase, is a logical choice for treatment of SSADH deficiency because it will prevent the conversion of GABA to GHB. Outcomes have been mixed, and in selected patients treatment has led to deterioration (Good, 2011, J AAPOS. 15(5):411-2; Pellock, 2011, Acta Neurol Scand Suppl. 192:83-91; Escalera et al., 2010, An Pediatr (Barc). 72(2):128-32; Casarano et al., 2011, JIMD Rep. 2:119-23; Matern et al., 1996, J Inherit Metab Dis. 19(3):313-8; Al-Essa et al., Brain Dev. 2000, 22(2):127-31. 2000). Targeted therapy for SSADH deficiency remains elusive and interventions palliative.

Thus, in some embodiments, the present invention provides a method of treating SSADHD in a patient in need thereof, comprising administering to said patient a compound of formula A or a pharmaceutically acceptable salt thereof.

### 5. Pharmaceutically Acceptable Compositions

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention are administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of the invention can also be administered topically, such as directly to the eye, e.g., as an eye-drop or ophthalmic ointment. Eye drops typically comprise an effective amount of at least one compound of the invention and a carrier capable of being safely applied to an eye. For example, the eye drops are in the form of an isotonic solution, and the pH of the solution is adjusted so that there is no irritation of the eye. In many instances, the epithelial barrier interferes with penetration of molecules into the eye. Thus, most currently used ophthalmic drugs are supplemented with some form of penetration enhancer. These penetration enhancers work by loosening the tight junctions of the most superior epithelial cells (Burstein, 1985, Trans Ophthalmol Soc UK 104(Pt 4):402-9; Ashton et al., 1991, J Pharmacol Exp Ther. 259(2):719-24; Green et al., 1971, Am J Ophthalmol. 72(5):897-905). The most commonly used penetration enhancer is benzalkonium chloride (Tang et al., 1994, J Pharm Sci. 83(1):85-90; Burstein et al, 1980, Invest Ophthalmol Vis Sci. 19(3):308-13), which also works as preservative against microbial contamination. It is typically added to a final concentration of 0.01-0.05%.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1: General Reaction Sequence of Certain Compounds of Formula II

Aldehyde trapping agents were made as described in US patent publication no. US 2013/0190500, published Jul. 25, 2013, which is hereby incorporated by reference, as indicated in the Scheme 1. “R” represents an optionally substituted group on U as defined above, and “n” represents the number of occurances of said optionally substituted groups. Exemplary such methods are described further below.

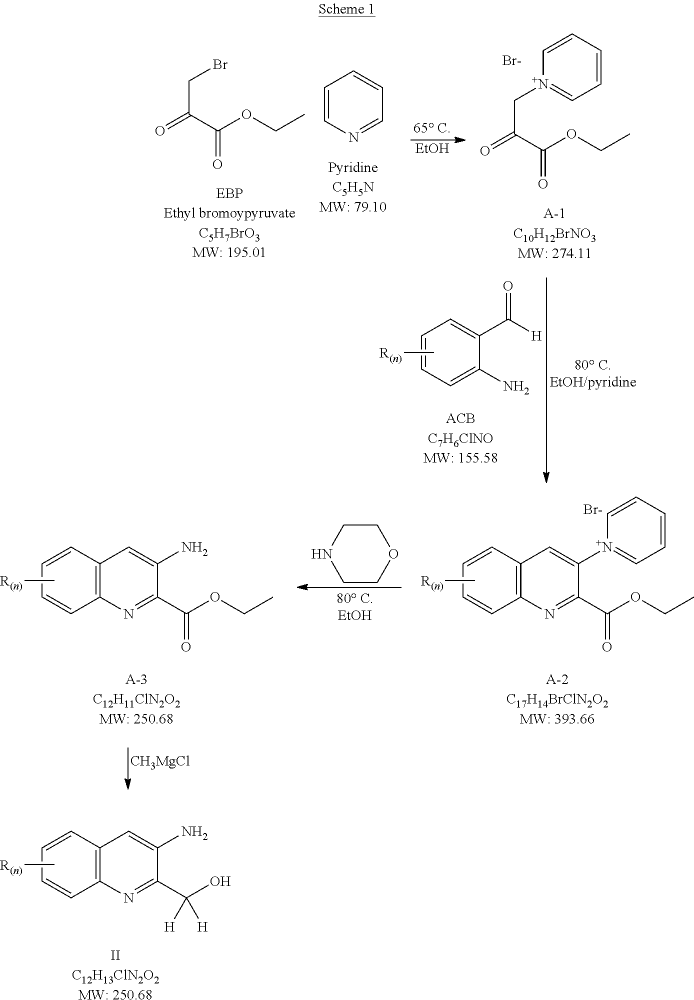

### Example 2: Synthesis of II-5

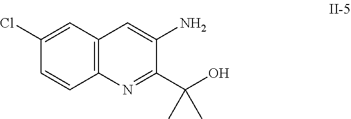 [SMILES: CC(C)(O)c1nc2ccc(Cl)cc2cc1N]

Synthesis of 1-(3-ethoxy-2,3-dioxopropyl)pyridin-1-ium bromide (A-1). To a 2 L round bottom flask was charged ethanol (220 mL) and pyridine (31 g, 392 mmol), and the resulting solution was stirred at a moderate rate of agitation under nitrogen. To this solution was added ethyl bromopyruvate (76.6 g, 354 mmol) in a slow, steady stream. The reaction mixture was allowed to stir at 65±5° C. for 2 hours.

Synthesis of 1-(6-chloro-2-(ethoxycarbonyl)quinolin-3-yl)pyridin-1-ium bromide (A-2). Upon completion of the 2 hour stir time in the previous reaction, the reaction mixture was slowly cooled to 18-22° C. The flask was vacuum-purged three times at which time 2-amino-5-chloro-benzaldehyde (ACB) (50.0 g, 321 mmol) was added directly to the reaction flask as a solid using a long plastic funnel. Pyridine (64.0 g, 809 mmol) was added followed by an EtOH rinse (10 mL) and the reaction mixture was heated at 80±3° C. under nitrogen for about 16 hours (overnight) at which time HPLC analysis indicated that the reaction was effectively complete.

Synthesis of ethyl 3-amino-6-chloroquinoline-2-carboxylate (A-3). The reaction mixture from the previous reaction was cooled to about 70° C. and morpholine (76.0 g, 873 mmol)) was added to the 2 L reaction flask using an addition funnel. The reaction mixture was heated at 80±2° C. for about 2.5 hours at which time the reaction was considered complete by HPLC analysis (area % of A-3 stops increasing). The reaction mixture was cooled to 10-15° C. for the quench, work up, and isolation.

To the 2 L reaction flask was charged water (600 g) using the addition funnel over 30-60 minutes, keeping the temperature below 15° C. by adjusting the rate of addition and using a cooling bath. The reaction mixture was stirred for an additional 45 minutes at 10-15° C. then the crude A-3 was isolated by filtration using a Buchner funnel. The cake was washed with water (100 mL×4) each time allowing the water to percolate through the cake before applying a vacuum. The cake was air dried to provide crude A-3 as a nearly dry brown solid. The cake was returned to the 2 L reaction flask and heptane (350 mL) and EtOH (170 mL) were added, and the mixture heated to 70±3° C. for 30-60 minutes. The slurry was cooled to 0-5° C. and isolated by filtration under vacuum. The A-3 was dried in a vacuum drying oven under vacuum and 35±3° C. overnight (16-18 hours) to provide A-3 as a dark green solid.

Synthesis of 2-(3-amino-6-chloroquinolin-2-yl)propan-2-ol (II-5). To a 2 L round bottom flask was charged methylmagnesium chloride (200 mL of 3.0 M solution in THF, 600 mmol). The solution was cooled to 0-5° C. using an ice bath.

A 500 mL flask (magnetic stirring) was charged with 22.8 grams A-3 from the previous reaction and THF (365 mL), stirred to dissolve, and then transferred to an addition funnel on the 2 L Reaction Flask. The A-3 solution was added drop-wise to the reaction flask over 5.75 hours, keeping the temperature of the reaction flask between 0-5° C. throughout the addition. At the end of the addition the contents of the flask were stirred for an additional 15 minutes at 0-5° C. then the cooling bath was removed and the reaction was allowed to stir overnight at ambient temperature.

The flask was cooled in an ice bath and the reaction mixture was carefully quenched by adding EtOH (39.5 g, 857 mmol) drop-wise to the reaction mixture, keeping the temperature of the reaction mixture below 15° C. during the course of the addition. An aqueous solution of NH₄Cl (84.7 g NH₄Cl in 415 mL water) was then carefully added and the mixture stirred under moderate agitation for about 30 minutes then transferred to a separatory funnel to allow the layers to separate. Solids were present in the aqueous phase so HOAc (12.5 g) was added and the contents swirled gently to obtain a nearly homogeneous lower aqueous phase. The lower aqueous layer was transferred back to the 2 L reaction flask and stirred under moderate agitation with 2-methylTHF (50 mL) for about 15 minutes. The original upper organic layer was reduced in volume to approximately 40 mL using a rotary evaporator at ≤40° C. and vacuum as needed. The phases in the separatory funnel were separated and the upper 2-MeTHF phase combined with the product residue, transferred to a 500 mL flask, and vacuum distilled to an approximate volume of 25 mL. To this residue was added 2-MeTHF (50 mL) and distilled to an approximate volume of 50 mL. The crude compound II-5 solution was diluted with 2-MeTHF (125 mL), cooled to 5-10° C., and 2M H₂SO₄ (aq) (250 mL) was slowly added and the mixture stirred for 30 minutes as the temperature was allowed to return to ambient. Heptane (40 mL) was charged and the reaction mixture stirred for an additional 15 minutes then transferred to a separatory funnel, and the layers were allowed to separate. The lower aqueous product layer was extracted with additional heptane (35 mL), then the lower aqueous phase was transferred to a 1 L reaction flask equipped with a mechanical stirrer, and the mixture was cooled to 5-10° C. The combined organic layers were discarded. A solution of 25% NaOH (aq) was prepared (NaOH, 47 g, water, 200 mL) and slowly added to the 1 L reaction flask to bring the pH to a range of 6.5-8.5.

EtOAc (250 mL) was added and the mixture was stirred overnight. The mixture was transferred to a separatory funnel and the lower phase discarded. The upper organic layer was washed with brine (25 mL), then the upper organic product layer was reduced in volume on a rotary evaporator to obtain the crude compound II-5 as a dark oil that solidified within a few minutes. The crude compound II-5 was dissolved in EtOAc (20 mL) and filtered through a plug of silica gel (23 g) eluting with 3/1 heptane/EtOAc until all compound II-5 was eluted (approximately 420 mL required) to remove most of the dark color of compound II-5. The solvent was removed in vacuo to provide 14.7 g of compound II-5 as a tan solid. Compound II-5 was taken up in EtOAc (25 mL) and eluted through a column of silica gel (72 g) using a mobile phase gradient of 7/1 heptane/EtOAc to 3/1heptane/EtOAc (1400 mL total). The solvent fractions containing compound II-5 were stripped. Compound II-5 was diluted with EtOAc (120 mL) and stirred in a flask with Darco G-60 decolorizing carbon (4.0 g) for about 1 hour. The mixture was filtered through celite using a fitted funnel, rinsing the cake with EtOAc (3×15 mL). The combined filtrates were stripped on a rotary evaporator and compound II-5 dissolved in heptane (160 mL)/EtOAc (16 mL) at 76° C. The homogeneous solution was slowly cooled to 0-5° C., held for 2 hours, then compound II-5 was isolated by filtration. After drying in a vacuum oven for 5 hours at 35° C. under best vacuum, compound II-5 was obtained as a white solid.

HPLC purity: 100% (AUC)
HPLC (using standard conditions):
A-2: 7.2 minutes
A-3: 11.6 minutes

### Synthesis of 2-amino-5-chlorobenzaldehyde (ACB)

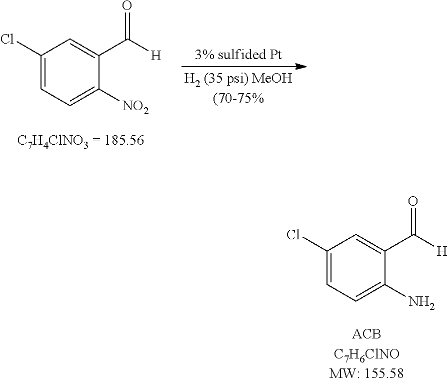 [SMILES: O=Cc1cc(Cl)ccc1[N+](=O)[O-] ; Nc1ccc(Cl)cc1C=O]

After a N₂ atmosphere had been established and a slight stream of N₂ was flowing through the vessel, platinum, sulfided, 5 wt % on carbon, reduced, dry (9.04 g, 3.0 wt % vs the nitro substrate) was added to a 5 L heavy walled pressure vessel equipped with a large magnetic stir-bar and a thermocouple. MeOH (1.50 L), 5-chloro-2-nitrobenzaldehyde (302.1 g, 1.63 mol), further MeOH (1.50 L) and Na₂CO₃ (2.42 g, 22.8 mmol, 0.014 equiv) were added. The flask was sealed and stirring was initiated at 450 rpm. The solution was evacuated and repressurized with N₂ (35 psi), 2×. The flask was evacuated and repressurized with H₂ to 35 psi. The temperature of the solution reached 30° C. w/in 20 min. The solution was then cooled with a water bath. Ice was added to the water bath to maintain a temperature below 35° C. Every 2 h, the reaction was monitored by evacuating and repressurizing with N₂ (5 psi), 2× prior to opening. The progress of the reaction could be followed by TLC: 5-Chloro-2-nitrobenzaldehyde (R_{f}=0.60, CH₂Cl₂, UV) and the intermediates (R_{f}=0.51, CH₂Cl₂, UV and R_{f}=0.14, CH₂Cl₂, UV) were consumed to give ACB (R_{f}=0.43, CH₂Cl₂, UV). At 5 h, the reaction had gone to 98% completion (GC), and was considered complete. To a 3 L medium fritted funnel was added celite (ca. 80 g). This was settled with MeOH (ca. 200 mL) and evaporated under vacuum. The reduced solution was transferred via cannula into the funnel while gentle vacuum was used to pull the solution through the celite plug. This was chased with MeOH (150 mL 4×). The solution was transferred to a 5 L three-necked round-bottom flask. At 30° C. on a rotavap, solvent (ca. 2 L) was removed under reduced pressure. An N2 blanket was applied. The solution was transferred to a 5 L four-necked round-bottomed flask equipped with mechanical stirring and an addition funnel. Water (2.5 L) was added dropwise into the vigorously stirring solution over 4 h. The slurry was filtered with a minimal amount of vacuum. The collected solid was washed with water (1.5 L 2×), iPA (160 mL) then hexanes (450 mL 2×). The collected solid (a canary yellow, granular solid) was transferred to a 150×75 recrystallizing dish. The solid was then dried under reduced pressure (26-28 in Hg) at 40° C. overnight in a vacuum-oven. ACB (>99% by HPLC) was stored under a N₂ atmosphere at 5° C.

### Example 3: General Reaction Sequence for Certain Compounds of Formula II

The following aldehyde trapping agents were made as described the '836 publication. Exemplary methods are described further below.

### Example 4: Synthesis of II-7

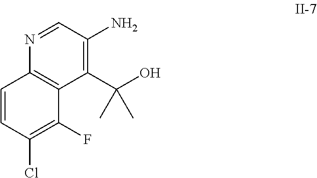 [SMILES: CC(C)(O)c1c(N)cnc2ccc(Cl)c(F)c12]

Synthesis of (E)- and (Z)-3-chloro-2-fluoro-6-(2-nitrovinylamino)benzoic acid (7-1). 37.19 g crude wet methazonic acid (prepared by the method of G. B. Bachman et al., J. Am. Chem. Soc. 69, 365-371 (1947)) was mixed with 50 g 6-amino-3-chloro-2-fluorobenzoic acid (Butt Park Ltd., Camelford, Cornwall, UK) and 750 mL acetone and shaken until a clear solution was formed. To the solution was added sequentially 200 mL water and 200 mL 12 N HCl, and the solution was kept 3 days at room temperature. The mixture was diluted with 2 L water and filtered. The filtrate was evaporated to remove acetone and filtered. The combined solids were washed with water (4×200 mL) and dried at 60° C. under high vacuum to afford 7-1 as a 4.5:1 mixture of E- and Z-isomers. ¹H NMR (400 MHz, DMSO-d₆) δ: E-isomer 6.79 (d, 1H, J=6.4 Hz), 7.58 (d, 1H, J=8.4 Hz), 7.83 (t, 1H, J=8.4 Hz), 7.99 (dd, 1H, J=6.4, 13.2 Hz), 12.34 (d, 1H, NH, J=13.2 Hz), 14.52 (br, 1H, OH). Z-isomer 7.39 (d, 1H, J=11.2 Hz), 7.42 (d, 1H, J=9.6 Hz), 7.71 (t, 1H, J=8.4 Hz), 8.49 (t, 1H, J=11.6 Hz), 10.24 (d, 1H, NH, J=12.4 Hz), 14.52 (br, 1H, OH). LC-MS: 259 [(M-H)⁻].

Synthesis of 6-chloro-5-fluoro-3-nitroquinolin-4-ol (7-2). A mixture of 62.0 g (7-1), 55.2 g N-ethyl-N′-(3-dimethylaminopropyl)carbodiimide hydrochloride (EDC) and 30.1 g N-hydroxysuccinimide (HOSu) in 1 L absolute dimethylformamide (DMF) was stirred at room temperature for 1 h. 4-dimethylaminopyridine (DMAP, 38.7 g) was added and the mixture was stirred at room temperature for 2 h. The mixture was filtered, and the solid was washed with 10% HOAc (4×200 mL), air-dried overnight, and then dried at 60° C. under high vacuum to give (7-2) as a pale yellow powder. ¹H NMR (400 MHz, DMSO-d₆) δ: 7.52 (dd, 1H, J=0.8, 8.8 Hz), 7.91 (dd, 1H, J=7.2, 8.8 Hz), 9.15 (s, 1H), 13.0 (br, 1H, OH). LC-MS: 242.9 (MH)⁺, 264.9 (MNa)⁺.

Synthesis of 4-bromo-6-chloro-5-fluoro-3-nitroquinoline (7-3). A mixture of 40 g (7-2) and 71 g POBr₃ in 150 mL dry DMF was stirred at 80° C. for 1 h. The mixture was cooled to room temperature, diluted with 2 L CH₂Cl₂, and transferred to a separatory funnel containing 1.5 L ice water. The organic layer was separated, washed with ice water (3×1.5 L), dried with MgSO₄, and evaporated to give crude (7-3) as a light brown solid, which was used without further purification. ¹H NMR (400 MHz, CDCl₃) δ: 4.70 (br, 2H, NH₂), 7.42 (dd, 1H, J=6.0, 9.0 Hz), 7.73 (dd, 1H, J=1.8, 8.8 Hz). LC-MS: 274.8 (MH)⁺, 276.8 [(M+2)H]⁺, 278.8 [(M+4)H]⁺.

Synthesis of 4-bromo-6-chloro-5-fluoroquinolin-3-amine (7-4). Crude (7-3) (51.2 g) was dissolved in 40 mL glacial HOAc under Ar, 3 g Fe powder was added, and the mixture was stirred at 60° C. for 10 min. The mixture was diluted with 200 mL EtOAc, filtered through Celite, and the Celite was washed thoroughly with EtOAc. The combined filtrates were passed through a short silica gel column, and the column was washed with EtOAc until all (7-4) was recovered. The combined EtOAc fractions were evaporated to dryness to give crude (7-4) which was crystallized from hexanes-EtOAc to provide (7-4) as a pale brown solid. ¹H NMR (400 MHz, CDCl₃) δ: 4.70 (br, 2H, NH₂), 7.42 (dd, 1H, J=6.0, 9.0 Hz), 7.73 (dd, 1H, J=1.8, 8.8 Hz). LC-MS: 274.8 (MH)⁺, 276.8 [(M+2)H]⁺, 278.8 [(M+4)H]⁺.

Synthesis of 2-(3-amino-6-chloro-5-fluoroquinolin-4-yl)propan-2-ol (II-7). A dry 1 L round bottom flask was flushed with argon and cooled to −78° C. in a dry ice/acetone bath. Dry tetrahydrofuran (THF, 300 mL) was injected, followed by 72.6 mL 2.5 M n-BuLi/hexanes. (7-4) (20 g) in 300 mL dry THF was added dropwise with vigorous stirring over 2 h, affording a dark red solution of the 4-quinolinelithium. Ultra dry acetone (27 mL) was added dropwise over 10 min, and the solution was stirred for an additional 10 min. A solution of 20 g NH₄Cl in 100 mL water was added and the mixture was warmed to room temperature, transferred to a separatory funnel containing 300 mL EtOAc, and shaken thoroughly. The organic layer was separated and the aqueous layer was extracted with EtOAc (2×250 mL). The combined organic layers were dried with anhydrous MgSO₄ and evaporated to a dark brown residue which was partially purified by chromatography on a silica gel column eluted with hexanes-EtOAc to afford a mixture containing 6-chloro-5-fluoroquinolin-3-amine and II-7. II-7 was isolated by crystallization from hexanes-EtOAc. ¹H NMR (400 MHz, CD₃OD) δ: 1.79 (s, 3H), 1.80 (s, 3H), 7.36 (dd, 1H, J=7.2, 8.8 Hz), 7.61 (dd, 1H, J=1.6, 9.0 Hz), 8.35 (s, 1H). ¹³C NMR (100 MHz, CD₃OD) δ: 29.8, 29.9, 76.7, 120.4 (d, J_{C-F}=12 Hz), 120.5 (d, J_{C-F}=4 Hz), 125.4, 126.1 (d, J_{C-F}=3 Hz), 126.6 (d, J_{C-F}=3 Hz), 143.1, 143.2 (d, J_{C-F}=5 Hz), 148.3, 152.7 (d, J_{C-F}=248 Hz). LC-MS: 254.9 (MH)⁺, 256.9 [(M+2)H]⁺.

### Example 5: Synthesis of II-8

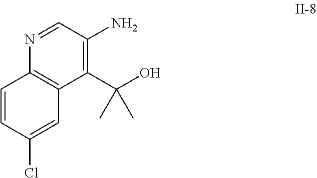 [SMILES: CC(C)(O)c1c(N)cnc2ccc(Cl)cc12]

Synthesis of 6-chloro-3-nitroquinolin-4-ol (8-1). A mixture of cis- and trans-5-chloro-2-(2-nitrovinylamino)benzoic acid (68.4 g, Sus et al., Liebigs Ann. Chem. 583: 150 (1953)), 73 g EDC and 35.7 g HOSu in 1 L dry DMF was stirred at room temperature for 1 h. After adding 45.8 g DMAP the mixture was stirred at room temperature for 2 h. To the stirred mixture was slowly added 1 L 10% HOAc, and the resulting suspension was poured into 2 L 10% HOAc. The solid was filtered off, washed with 10% HOAc (4×400 mL) and dried at 80° C. under high vacuum to give (8-1) as a tan powder.

Synthesis of 4-bromo-6-chloro-quinolin-3-amine (8-2). A mixture of 25 g (8-1) and 50 g POBr₃ in 100 mL dry DMF was stirred at 80° C. for 1 h. The reaction mixture was cooled to room temperature, diluted with 2 L CH₂Cl₂, and transferred to a separatory funnel containing 1 L ice water. The organic layer was separated, washed with ice water (3×1 L), dried with MgSO₄, and evaporated to provide crude 4-bromo-6-chloroquinolin-4-ol as a light brown solid (38 g, 100% crude yield). The quinolinol was dissolved in 750 mL glacial HOAc, 36 g iron powder was added, and the stirred mixture was heated under Ar at 60° C. until the color turned to grey. The mixture was diluted with 2 L EtOAc, filtered through Celite, and the Celite was washed with EtOAc. The combined filtrates were passed through a short silica gel column which was washed with EtOAc until all (8-2) was recovered. The combined fractions were evaporated to dryness and the residue was crystallized from hexanes-EtOAc to provide (8-2) as a tan solid.

¹H NMR (400 MHz, CDCl₃) δ: 4.47 (br, 2H, NH₂), 7.41 (dd, 1H, J=2.4, 8.8 Hz), 7.89 (d, 1H, J=9.2 Hz), 7.96 (d, 1H, J=2.4 Hz), 8.45 (s, 1H). LC-MS: 256.7 (MH)⁺, 258.7 [(M+2)H]⁺, 260.7 [(M+4)H]⁺.

Synthesis of 2-(3-amino-6-chloroquinolin-4-yl)propan-2-ol (II-8). A mixture of 20 g (8-2) and 800 mL dioxane was stirred at 60° C. until a solution formed, cooled to room temperature, and sparged with dry HCl for 5 min. The solvent was evaporated, and 500 mL dioxane was added and evaporated to provide 4-bromo-6-chloroquinolin-3-aminium hydrochloride. The product was mixed with 100 g NaI and 600 mL dry MeCN and refluxed overnight. The solvent was evaporated and the residue was partitioned between 500 mL EtOAc and a solution of 10 g NaHCO₃ in 500 mL water. The organic layer was separated, and the aqueous layer was extracted with EtOAc (2×200 mL). The combined organic layers were dried with MgSO₄ and evaporated to provide 6-chloro-4-iodoquinolin-3-amine as a tan solid. A dry 1 L round bottom flask was flushed with Ar and cooled to −78° C. in a dry ice/acetone bath. Dry THF (350 mL) was added followed by 188 mL 1.7 M t-BuLi/pentane with vigorous stirring. A solution of 25.8 g crude 6-chloro-4-iodoquinolin-3-amine in 350 mL dry THF was added dropwise to the stirred mixture. When addition was complete the reaction mixture was stirred at −78° C. for 5 min. Ultra dry acetone (50 mL) was added dropwise and the solution was stirred at −78° C. for 10 min after addition was complete. A solution of 20 g NH₄Cl in 200 mL water was added and the mixture was warmed up to room temperature, transferred to a separatory funnel containing 300 mL EtOAc. The organic layer was separated and the aqueous layer extracted with EtOAc (2×250 mL). The combined organic layers were dried with MgSO₄ and evaporated to a dark brown residue. The residue was partially purified by column chromatography on silica gel eluted with hexanes-EtOAc. All fractions containing (8-3) were combined and evaporated to give crude (8-3) as a red oil.

A batch of crude ii) (ca. 2 g) obtained from a separate synthesis was added to this product, and the combined batches were dissolved in 50 mL EtOAc and filtered. The filtrate and washings were combined and concentrated to an oil which was diluted with 10 mL hot hexanes, treated dropwise with EtOAc until a clear solution formed, and allowed to evaporate at room temperature overnight in the fume hood. The oily mother liquor was removed and the solid was washed with minimum volumes of 3:1 hexanes-EtOAc. After recrystallization twice from hexanes-EtOAc, a first crop of pure (II-8) was obtained as off-white crystals. All the mother liquor and washings were pooled and EtOAc (ca. 50 mL) was added to form a clear solution which was extracted with 0.5 N aq. HCl (4×100 mL). The aqueous layers were pooled and neutralized with 20% NaOH to pH 8. The resulting suspension was extracted with EtOAc (3×50 mL) and the combined organic layers were dried with MgSO₄ and evaporated to dryness. The residue was purified by column chromatography and two crystallizations from hexanes-EtOAc to provide a second crop of (8-3). A third crop (8-3) was obtained by fractional crystallization of the combined mother liquor and washings from hexanes-EtOAc. ¹H NMR (400 MHz, CDCl₃) δ: 1.93 (s, 6H), 3.21 (br, 1H, OH), 5.39 (br, 2H, NH₂), 7.29 (dd, 1H, J=2.0, 8.8 Hz), 7.83 (d, 1H, J=8.8 Hz), 7.90 (d, 1H, J=2.0 Hz), 8.21 (s, 1H). ¹³C NMR (100 MHz, CDCl₃) δ: 31.5, 76.5, 123.2, 124.6, 125.7, 127.5, 131.5, 131.9, 138.8, 141.5, 146.5. LC-MS: 236.9 (MH)⁺, 238.9 [(M+2)H]⁺.

### Example 6: Synthesis of II-39

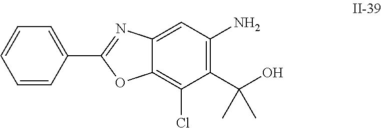 [SMILES: CC(C)(O)c1c(N)cc2nc(-c3ccccc3)oc2c1Cl]

Synthesis of 4-Benzoylamino-5-hydroxy-2-nitrobenzoic acid ethyl ester (39-1). A mixture of 2.26 g crude 4-amino-5-hydroxy-2-nitrobenzoic acid ethyl ester (40-4, see below) and 1.91 g benzoyl chloride in 25 mL 1,4-dioxane was stirred at 95° C. for 1 h. The solvent was removed and the residue was evaporated twice with EtOH. The residue was further evaporated twice with EtOAc, and then was dried at 60° C. under high vacuum to give crude (39-1) as a tan solid.

Synthesis of 4-Benzoylamino-2-chloro-3-hydroxy-6-nitrobenzoic acid ethyl ester (39-2). A suspension of 3.23 g (39-1) in 100 mL dioxane was stirred until a clear solution was formed. To the solution was added 70 μL DIPA, and the solution was stirred to 50° C., followed by addition of 2.03 mL SO₂Cl₂. The reaction mixture was stirred under argon at 50° C. for 1 h, cooled to room temperature, diluted with 200 mL EtOAc, washed with water (3×100 mL), and then dried with MgSO₄. The solvent was evaporated and the residue was dried at 60° C. under high vacuum to give crude (39-2) as a brown solid.

Synthesis of 7-Chloro-5-nitro-2-phenylbenzoxazole-6-carboxylic acid ethyl ester (39-3). A mixture of crude 3.74 g (39-2) and 3.93 g Ph₃P in 50 mL dry THF was stirred at room temperature until a solution was formed. To the solution was added 6.7 mL 40% DEAD/toluene, and the mixture was stirred at 70° C. for 1 h. The mixture was diluted with EtOH and evaporated. The residue was separated by silica gel column chromatography with hexane-EtOAc as eluent to give (39-3) as a white solid.

Synthesis of 5-Amino-7-chloro-2-phenylbenzoxazole-6-carboxylic acid ethyl ester (39-4). A mixture of 0.89 g (39-3), 2.0 g iron powder and 25 mL glacial HOAc was heated at 60° C. under vigorous stirring for 1.5 h. The mixture was diluted with 200 mL EtOAc. The slurry was passed through a celite pellet, and the celite was washed with EtOAc. The combined filtrates were pass through a short silica gel column, and the column was eluted with EtOAc. The combined yellow fractions were evaporated, and the residue was crystallized from hexanes-EtOAc to give pure (39-4) as a bright yellow solid.

Synthesis of 2-(5-Amino-7-chloro-2-phenylbenzoxazol-6-yl)propan-2-ol (II-39). A mixture of 6 mL 3.0 M MeMgCl/THF and 6 mL THF was protected under argon, and cooled in an ice bath with vigorous stirring. To it was added dropwise a solution of 638 mg (39-4) in 50 mL THF. After complete addition, the mixture was stirred at 0° C. for 5 min. To the mixture was added 100 mL saturated NH₄Cl with cooling and vigorous stirring. The organic layer was separated, and the aqueous layer was extracted with DCM (3×100 mL). The combined organic layers were dried with MgSO₄ and evaporated. The crude product was purified by silica gel column chromatography with MeOH-DCM as eluent, and then crystallized from heptane-DCM to give pure (II-39) as a pale yellow solid. ¹H NMR (400 MHz, CDCl₃) δ: 1.92 (s, 6H), 4.69 (br, 3H, NH₂ and OH), 6.87 (s, 1H), 7.48-7.54 (3H), 8.21 (m, 2H). ¹³C NMR (100 MHz, CDCl₃) δ: 31.0, 77.0, 106.3, 113.6, 126.8, 126.9, 127.7, 128.9, 131.6, 140.9, 143.0, 145.4, 163.9. LC-MS: 303.1 (MH)⁺, 305.0 [(M+2)H]⁺.

### Example 7: Synthesis of II-40

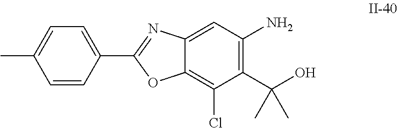 [SMILES: Cc1ccc(-c2nc3cc(N)c(C(C)(C)O)c(Cl)c3o2)cc1]

Synthesis of 3-Methoxy-4-(trifluoroacetylamino)benzoic acid (40-1). To a suspension of 5.0 g 4-amino-3-methoxybenzoic acid in 200 mL EtOAc was added under stirring a solution of 5.0 mL (CF₃CO)₂O in 50 mL of EtOAc. After complete addition, the reaction mixture was further stirred at room temperature for 2 h. The solution was filtered, and the filtrate was evaporated to dryness. The residue was dissolved and evaporated twice in EtOAc. The final residue was dried under high vacuum to afford pure (40-1) as a white solid.

Synthesis of 5-Methoxy-2-nitro-4-(trifluoroacetylamino)benzoic acid (40-2). A suspension of 7.55 g (40-1) in 80 mL 96% H₂SO₄ was stirred at room temperature until a homogeneous solution was formed. The solution was cooled with an ice bath under stirring while a solution of 2.03 g 90.6% fuming HNO₃ in 20 mL 96% H₂SO₄ was added dropwise under cooling. The temperature was maintained below 10° C. After complete addition, the mixture was further stirred for 10 min, and then slowly added onto 200 g ice under vigorous stirring. The mixture was saturated with NaCl and extracted with EtOAc (3×100 mL). The combined organic layer was washed with brine (2×50 mL), dried with Na₂SO₄, and then evaporated to give pure (40-2) as a light brown solid.

Synthesis of 4-Amino-5-hydroxy-2-nitrobenzoic acid (40-3). A mixture of 6.94 g (40-2) in 35 mL 20% aqueous NaOH was stirred under argon at 100° C. overnight. The mixture was cooled to room temperature. To it was added dropwise 20 mL 12 N HCl under ice bath cooling. After complete addition, the solution was evaporated, and the residue was extracted with 200 mL absolute EtOH. The solid NaCl was filtered off, and the filtrate was evaporated to give the crude HCl salt of (40-3) as a dark grey solid.

Synthesis of 4-Amino-5-hydroxy-2-nitrobenzoic acid ethyl ester (40-4). The above 6.95 g crude HCl salt of (40-3) was dissolved in 250 mL absolute EtOH. The solution was purged with dry HCl to nearly saturation, and then stirred at 80° C. for 36 h. The solvent was evaporated, and the residue was partitioned between 200 mL EtOAc and 200 mL brine. The aqueous layer was extracted with EtOAc (2×100 mL). The combined organic layer was dried with Na₂SO₄, acidified with 2 mL of HOAc, and then passed through a short silica gel column. The column was eluted with 1% HOAc/EtOAc. The combined yellow fraction was evaporated to give crude (40-4) as a red viscous oil.

Synthesis of 5-Hydroxy-4-(4-methylbenzoylamino)-2-nitrobenzoic acid ethyl ester (40-5). A mixture of 2.26 crude (40-4) and 2.1 g p-toluoyl chloride in 25 mL 1,4-dioxane was stirred at 95° C. for 1.5 h. The solvent was removed, and the residue was evaporated twice with EtOH and then evaporated twice with EtOAc. The final residue was dried at 60° C. under high vacuum to give crude (40-5) as a tan solid.

Synthesis of 2-Chloro-3-hydroxy-4-(4-methylbenzoylamino)-6-nitrobenzoic acid ethyl ester (40-6). A suspension of 3.35 g (40-5) in 100 mL dioxane was stirred until a clear solution was formed, and then 70 μL diisopropylamine (DIPA) was added. The solution was stirred at 50° C. while 1.96 mL SO₂Cl₂ was added. The reaction mixture was stirred under argon at 50° C. for 1 h, cooled to room temperature, diluted with 200 mL EtOAc, washed with water (3×100 mL), and dried with MgSO₄. The solvent was evaporated and the residue was dried at 60° C. under high vacuum to give crude (40-6) as a brown solid.

Synthesis of 7-Chloro-5-nitro-2-(p-tolyl)benzoxazole-6-carboxylic acid ethyl ester (40-7). A mixture of 4.35 g crude (40-6) and 3.93 g Ph₃P in 50 mL dry THF was stirred at room temperature until a solution was formed. To the solution was added 6.7 mL 40% DEAD/toluene, and the mixture was stirred at 70° C. for 1 h. The mixture was diluted with 50 mL EtOH and evaporated. The residue was separated by silica gel column chromatography with hexane-EtOAc as eluent to give pure (40-7) as a white solid.

Synthesis of 5-Amino-7-chloro-2-(p-tolyl)benzoxazole-6-carboxylic acid ethyl ester (40-8). A mixture of 1.17 g (40-7), 1.07 g iron powder and 25 mL glacial HOAc was heated at 60° C. under vigorous stirring for 3 h. The reaction mixture was diluted with 200 mL EtOAc. The slurry was passed through a celite pellet, and the celite was washed with EtOAc. The combined filtrates were passed through a short silica gel column, and the column was eluted with EtOAc. The combined yellow fractions were evaporated, and the residue was crystallized from hexanes-EtOAc to give pure (40-8) as a bright yellow solid.

Synthesis of 2-(5-Amino-7-chloro-2-(p-tolyl)benzoxazol-6-yl)propan-2-ol (II-40). A mixture of 7.0 mL 3.0 M MeMgCl/THF and 6 mL THF was protected under argon, and cooled in an ice bath with vigorous stirring. To it was added dropwise a solution of 886 mg (40-8) in 50 mL THF. After compete addition, the mixture was stirred at 0° C. for 5 min. To the mixture was added 100 mL saturated NH₄Cl with ice bath cooling and vigorous stirring. The organic layer was separated, and the aqueous layer was extracted with CH₂Cl₂ (DCM) (3×100 mL). The combined organic layers were dried with MgSO₄ and evaporated. The crude product was purified by silica gel column chromatography with MeOH-DCM as eluent and then crystallized from heptane/DCM to give pure (II-40) as an off-white solid. ¹H NMR (400 MHz, CDCl₃) δ: 1.89 (s, 6H), 2.41 (s, 3H), 4.45 (br, 3H, NH₂ and OH), 6.81 (s, 1H), 7.27 (d, 1H, J=8.8 Hz), 8.07 (d, 1H, J=8.4 Hz). ¹³C NMR (100 MHz, CDCl₃) δ: 21.7, 31.0, 76.9, 106.2, 113.5, 124.0, 126.8, 127.6, 129.6, 140.9, 142.2, 142.9, 145.3, 164.1. LC-MS: 317.0 (MH)⁺, 319.0 [(M+2)H]⁺.

### Example 8: Synthesis of II-41

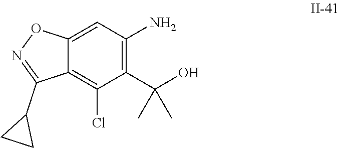 [SMILES: CC(C)(O)c1c(N)cc2onc(C3CC3)c2c1Cl]

Synthesis of (2-Chloro-4,6-dimethoxyphenyl)cyclopropylmethanone (41-1). A solution of 28.28 g 1-chloro-3,5-dimethoxybenzene and 17.8 mL cyclopropanecarbonyl chloride in 300 mL dry 1,2-dichloroethane (DCE) was protected with argon, and cooled in a dry ice/acetone bath to −30 to −40° C. To it was added in portions 32.4 g AlCl₃ powder under vigorous stirring. After complete addition, the solution was stirred at −30 to −40° C. for 30 min, and then allowed to warm up to room temperature. After further stirring at room temperature for 20 min, the mixture was added onto 1 kg ice under stirring. The mixture was extracted with ether (3×300 mL). The combined organic layers were dried with MgSO₄ and evaporated. The residue was separated by column chromatography with hexanes/EtOAc as eluent to give pure (41-1) as a white solid.

Synthesis of (2-Chloro-6-hydroxy-4-methoxyphenyl)cyclopropylmethanone (41-2). A solution of 13.45 g (41-1) in 100 mL dry DCM was protected with argon, and cooled at −78° C. (dry ice/acetone bath) under stirring. To it was added 62 mL 1 M BBr₃/DCM. After complete addition, the mixture was further stirred at −78° C. for 1 h. To the mixture was slowly injected 50 mL MeOH under dry ice/acetone bath cooling and vigorous stirring. After complete injection, the mixture was further stirred at −78° C. for 10 min, and then allowed to warm up to room temperature. The mixture was partitioned between 500 mL DCM and 500 mL brine. The organic layer was separated, washed with brine (2×100 mL), and then mixed with a solution of 4.0 g NaOH in 300 mL water. After stirring at room temperature for 1 h, the mixture was acidified with 10 mL 12 N aqueous HCl with stirring. The organic layer was separated, dried with MgSO₄, and evaporated. The residue was separated by silica gel column chromatography with hexanes-EtOAc as eluent to give (41-2) as a white solid.

Synthesis of (E)- and (Z)-(2-Chloro-6-hydroxy-4-methoxyphenyl)cyclopropylmethanone oxime (41-3). A mixture of 10.38 g (41-2) and 15.95 g NH₂OH.HCl in 150 mL dry pyridine was protected under argon, and stirred at 80° C. for 20 h. The solvent was evaporated, and the residue was partitioned between 400 mL 0.1 N HCl/brine and 400 mL Et₂O. The organic layer was separated, washed with water (2×50 mL), dried with MgSO₄ and evaporated. The residue was crystallized from heptane-EtOAc to give pure (41-3) as a white solid.

Synthesis of (E)- and (Z)-(2-Chloro-6-hydroxy-4-methoxyphenyl)cyclopropylmethanone O-acetyl oxime (41-4). To a suspension of 9.75 g (41-3) in 40 mL EtOAc was added 6.5 mL Ac₂O under stirring at room temperature. After complete addition, the mixture was stirred at room temperature for 1 h. To the mixture was added 50 mL MeOH and 20 mL pyridine, and the mixture was stirred at room temperature for 30 min. The solvent was evaporated, and the residue was partitioned between 300 mL 1 N HCl/brine and 300 mL EtOAc. The organic layer was separated, washed with water (2×50 mL), dried with MgSO₄ and evaporated to give crude (41-4) as a light brown oil.

Synthesis of 4-Chloro-3-cyclopropyl-6-methoxybenzisoxazole (41-5). Crude (41-4) was protected under argon, and heated in an oil bath at 150° C. for 3 h. The crude product was purified by silica gel column chromatography using hexanes-EtOAc as eluent to give pure (41-5) as a light tan solid.

Synthesis of 4-Chloro-3-cyclopropylbenzisoxazol-6-ol (41-6). A solution of 7.61 g (41-5) in 75 mL dry DCM was protected under argon, and cooled to −78° C. in a dry ice/acetone bath. To it was added dropwise 80 mL 1 M BBr₃ in DCM with vigorous stirring. After compete addition, the mixture was allowed to warm to room temperature, and then stirred at room temperature for 1 h. The mixture was again cooled to −78° C. in a dry ice/acetone bath. To the mixture was added 20 mL MeOH under vigorous stirring. After complete addition, the reaction mixture was allowed to warm to room temperature, and then partitioned between 1.5 L brine and 1.5 L EtOAc. The organic layer was separated, and the aqueous layer was extracted with EtOAc (2×300 mL). The combined organic layers were dried with MgSO₄, and passed through a short silica gel column that was eluted with EtOAc. The combined fractions were evaporated to give pure (41-6) as a light brown oil, which solidified upon standing.

Synthesis of 4-Chloro-3-cyclopropylbenzisoxazol-6-yl trifluoromethanesulfonate (41-7). A mixture of 6.88 g (41-6) and 4 mL pyridine in 50 mL DCM was protected under argon and stirred at 0° C. in an ice bath. To it was added dropwise 6.73 mL Tf₂O with vigorous stirring. After complete addition, the mixture was allowed to warm up to room temperature. After further stirring for 10 min at room temperature, the mixture was partitioned between 200 mL 1 N HCl and 300 mL DCM. The organic layer was separated, washed sequentially with 100 mL 1 N HCl, 100 mL brine, 100 mL 5% aqueous NaHCO₃ and 100 mL brine, dried with MgSO₄ and then evaporated. The residue was purified by column chromatography with hexanes-EtOAc as eluent to give pure (41-7) as an off-white solid.

Synthesis of tert-Butyl (4-chloro-3-cyclopropylbenzisoxazol-6-yl)carbamate (41-8). A mixture of 8.02 g (41-7), 2.87 g tert-butyl carbamate, 2.37 g tBuONa, 1.08 g tris(dibenzylideneacetone) dipalladium(0) (Pd₂dba₃), 2.0 g 2-di-tert-butylphosphino-2′,4′,6′-triisopropylbiphenyl (t-butyl Xphos) and 7 g 4 Å molecular sieves in 120 mL dry toluene was purged with argon, and then heated at 110° C. with vigorous stirring for 20 min. The reaction mixture was diluted with 300 mL EtOAc, and passed through a celite pellet which was then washed with EtOAc. The combined solutions were evaporated and the residue was separated by silica gel column chromatography with hexanes-EtOAc as eluent to give crude (41-8) as a light brown oil.

Synthesis of 6-Amino-4-chloro-3-cyclopropylbenzisoxazole (41-9). The 4.09 g crude (41-8) was dissolved in 10 mL DCM, followed by addition of 10 mL TFA. The mixture was stirred at room temperature for 30 min. The solvent was removed, and the residue was partitioned between 200 mL DCM and 200 mL 10% NaHCO₃. The organic layer was separated, washed with water (2×50 mL), dried with MgSO₄ and evaporated. The residue was separated by silica gel column chromatography with hexanes-EtOAc as eluent to give pure (41-9) as a white solid.

Synthesis of 5-Bromo-4-chloro-3-cyclopropylbenzisoxazol-6-ylamine (41-10) and 7-bromo-4-chloro-3-cyclopropylbenzisoxazol-6-ylamine (43-1, see below). To a solution of 1.96 g (41-9) in 100 mL DCM was added 1.67 g solid NBS in small portions under vigorous stirring at room temperature. After complete addition, the mixture was further stirred at room temperature for 30 min, diluted with 100 mL DCM, washed sequentially with 10% aqueous NaHSO₃ (200 mL) and water (2×200 mL), dried with MgSO₄, and evaporated to give a 1:1 mixture of (41-10) and (43-1) as a tan oil, which solidified on standing.

Synthesis of 6-Amino-4-chloro-3-cyclopropylbenzisoxazole-5-carbonitrile (41-11) and 6-amino-4-chloro-3-cyclopropylbenzisoxazole-7-carbonitrile (43-2, see below). A suspension of 2.72 g of a mixture of (41-10) and (43-1), 1.70 g CuCN and 3.62 g CuI in 25 mL dry DMF was purged with argon, and then heated at 110° C. in an oil bath with vigorous stirring for 15 h. The mixture was cooled to room temperature. To it was added 100 mL 30% aqueous NH₃. After stirring at room temperature for 1 h, the mixture was diluted with 300 mL water, and extracted with EtOAc (2×500 mL). The combined organic layers were washed with water (3×200 mL), dried with MgSO₄ and evaporated. The residue was separated by silica gel column chromatography with hexanes-EtOAc as eluent to give (41-11) as a light yellow solid, and (43-2) as a light tan solid.

Synthesis of 4-Chloro-5-cyano-3-cyclopropyl-6-(tritylamino)benzisoxazole (41-12). To a mixture of 435 mg (41-11) and 700 μL TEA in 20 mL DCM was added 1.09 g solid trityl chloride in small portions under stirring at room temperature. After complete addition, the mixture was further stirred at room temperature for 30 min. The reaction mixture was diluted with 300 mL DCM, washed with water (4×200 mL), dried with MgSO₄ and then evaporated. The residue was separated by silica gel column chromatography with DCM as eluent to give pure (41-12) as a white solid.

Synthesis of 4-Chloro-3-cyclopropyl-6-(tritylamino)benzisoxazole-5-carbaldehyde (41-13). A solution of 481 mg (41-12) in 13 mL dry THF was cooled in an ice bath with stirring. To the solution was added dropwise 7 mL 1 M DIBAL/toluene. After complete addition, the reaction mixture was stirred at 0° C. for 2.5 h. The reaction was quenched with 100 mL 1% aqueous tartaric acid, and the mixture was extracted with DCM (3×100 mL). The organic layer was washed with water (3×100 mL), dried with MgSO₄ and evaporated. The residue was dissolved in DCM and adsorbed onto silica gel. The mixture was air-dried and separated by silica gel column chromatography with hexanes-EtOAc as eluent to give crude (41-13) as a yellow solid.

Synthesis of 1-[4-Chloro-3-cyclopropyl-6-(tritylamino)benzisoxazol-5-yl]ethanol (41-14). The above 257.8 mg crude (41-13) was dissolved in 10 mL dry THF, and the solution was added to a mixture of 2.0 mL 3 M MeMgCl/THF and 2 mL dry THF at 0° C. (ice bath) with stirring. After complete addition, the mixture was further stirred at 0° C. for 5 min, and then quenched with 100 mL 5% NH₄Cl under ice bath cooling. The mixture was extracted with DCM (3×100 mL), dried with MgSO₄ and evaporated. The residue was separated by silica gel column chromatography with hexanes-EtOAc as eluent to give pure (41-14) as a white solid.

Synthesis of 1-[4-Chloro-3-cyclopropyl-6-(tritylamino)benzisoxazol-5-yl]ethanone (41-15). To a solution of 150.5 mg (41-14) in 20 mL dry DCM was added 271 mg solid Dess-Martin periodinane (1,1,1-triacetoxy-1,1-dihydro-1,2-benziodoxol-3(1H)-one, DMP) in small portions at room temperature under vigorous stirring. After complete addition, the reaction mixture was further stirred at room temperature for 10 min. The reaction mixture was diluted with 300 mL DCM, washed with water (4×200 mL), dried with MgSO₄ and evaporated. The residue was separated by silica gel column chromatography with hexanes-EtOAc as eluent to give pure (41-15) as a pale yellow solid.

Synthesis of 1-(6-Amino-4-chloro-3-cyclopropylbenzisoxazol-5-yl)ethanone (41-16). To a solution of 182 mg (41-15) in 20 mL dry DCM was added dropwise 2 mL TFA under stirring at room temperature. The solution was stirred at room temperature for 10 min, diluted with 200 mL DCM, washed with water (4×100 mL), dried with MgSO₄ and evaporated to give crude (41-16) as a white solid.

Synthesis of 2-(6-Amino-4-chloro-3-cyclopropylbenzisoxazol-5-yl)propan-2-ol (II-41). The 174.7 mg crude (41-16) was dissolved in 20 mL dry THF, and the solution was added dropwise to a well stirred mixture of 2.5 mL 3M MeMgCl/THF and 2 mL THF at 0° C. (ice bath). After complete addition, the mixture was further stirred at 0° C. for 5 min. To it was added dropwise 100 mL 5% aqueous NH₄Cl under ice bath cooling and stirring. The mixture was extracted with DCM (3×100 mL), dried with MgSO₄ and evaporated. The crude product was purified by silica gel column chromatography with MeOH-DCM as eluent and then crystallized from heptane-DCM to give pure (II-41) as a white solid. ¹H NMR (400 MHz, CDCl₃) δ: 1.10 (m, 2H), 1.20 (m, 2H), 1.91 (s, 6H), 2.18 (m, 1H), 4.28 (br, 2H, NH₂), 6.57 (s, 1H). ¹³C NMR (100 MHz, CDCl₃) δ: 8.68, 9.35, 30.0, 77.4, 97.4, 121.2, 125.1, 133.1, 145.7, 149.3, 166.4. LC-MS: 266.9 (MH)⁺, 269.0 [(M+2)H]⁺.

### Example 9: Synthesis of II-42

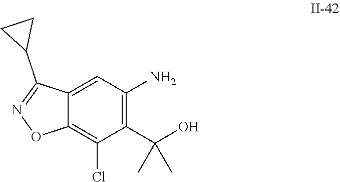 [SMILES: CC(C)(O)c1c(N)cc2c(C3CC3)noc2c1Cl]

Synthesis of cyclopropanecarboxylic acid methoxymethylamide (42-1). A suspension of 9.75 g N,O-dimethylhydroxylamine hydrochloride and 9.7 mL pyridine in 200 mL DCM was stirred at room temperature for 10 min, and then cooled in an ice bath with stirring. To the suspension was added dropwise a solution of 9.03 mL cyclopropanecarbonyl chloride in 40 mL DCM with vigorous stirring. After complete addition, the mixture was stirred at 0° C. for 30 min, and then at room temperature for 1 h. The solution was diluted with 100 mL DCM, washed with brine (3×200 mL), and dried with MgSO₄. The solvent was evaporated, and the residue vacuum distilled. The fraction collected at 43-45° C./1 mmHg gave (42-1) as a colorless liquid.

Synthesis of 2-(3-Chloro-4-fluorophenyl)-1,1,1,3,3,3-hexamethyldisilazane (42-2). A solution of 7.3 g 3-chloro-4-fluoroaniline in 100 mL dry THF was protected under argon and cooled at −78° C. (dry ice/acetone bath). To the solution was slowly added 21 mL 2.5 M nBuLi in hexanes with vigorous stirring. After complete addition, the suspension was further stirred at −78° C. for 10 min. To the latter was slowly added 6.65 mL chlorotrimethylsilane (TMSCl) under vigorous stirring. After complete addition, the mixture was further stirred at −78° C. for 30 min. To the latter was again added 24 mL 2.5 M nBuLi, followed by 7.65 mL TMSCl under vigorous stirring. The mixture was stirred at −78° C. for 30 min, and then allowed to warm to room temperature. The solvent was removed and the residue was vacuum distilled. The fractions collected below 95° C./1 mmHg were pooled to give (42-2) as a colorless liquid.

Synthesis of (5-Amino-3-chloro-2-fluorophenyl)(cyclopropyl)methanone (42-3). A solution of 9.11 g (42-2) in 100 mL dry THF was cooled to −78° C. in a dry ice/acetone bath under argon. To it was added dropwise 15.7 mL 2.5 M nBuLi in hexanes under vigorous stirring. After complete addition, the mixture was stirred at −78° C. for 2 h. To the mixture was added slowly 5.2 g (42-1) under stirring. After complete addition, the reaction mixture was stirred at −78° C. for 1 h, and then allowed to warm up to room temperature. The reaction mixture was poured into 400 mL cold 1:1 MeOH/1 N HCl under stirring. After further stirring for 30 min, the mixture was extracted with DCM (3×200 mL). The combined organic layers were dried with MgSO₄ and evaporated to give crude (42-3) as a light brown oil.

Synthesis of N-[3-Chloro-5-(cyclopropylcarbonyl)-4-fluorophenyl]acetamide (42-4). Crude (42-3) (6.09 g) was dissolved in 100 mL DCM. To it were added sequentially 6 mL acetic anhydride (Ac₂O) and 9.6 mL triethylamine (TEA) with ice bath cooling and vigorous stirring. After complete addition, the reaction mixture was further stirred at room temperature for 1 h, diluted with 200 mL DCM, and washed with 0.1 N HCl (3×200 mL). The organic layer was dried with MgSO₄ and evaporated. The crude product was purified by silica gel column chromatography with hexanes-EtOAc as eluent and then crystallized from hexanes-EtOAc to give pure (42-4) as a white solid.

Synthesis of (E)- and (Z)— N-{3-Chloro-5-[cyclopropyl(hydroxyimino)methyl]-4-fluorophenyl}acetamide (42-5). A mixture of 2.28 g (42-4), 3.1 g NH₂OH.HCl, 30 mL pyridine and 30 mL EtOH was stirred at 50° C. for 22 h. EtOH was evaporated, and the residue was partitioned between 200 mL Et₂O and 200 mL 1 N HCl/brine. The organic layer was separated, washed with water (2×20 mL), dried with MgSO₄ and evaporated to give pure (42-5) as an off-white amorphous solid.

Synthesis of N-(7-Chloro-3-cyclopropylbenzisoxazol-5-yl)acetamide (42-6). A solution of 2.01 g (42-5) in 40 mL dry DMF was protected with argon and stirred with ice bath cooling. To the solution was added in portions 1.48 g 60% NaH in mineral oil under vigorous stirring. After complete addition, the reaction mixture was stirred at room temperature for 1.5 h, and then was carefully added into a mixture of 300 mL saturated NaHCO₃ and 300 mL EtOAc under stirring. The organic layer was separated, washed with water (3×50 mL), dried with MgSO₄ and evaporated. The residue was separated by column chromatography with hexanes-EtOAc as eluent to give pure (42-6) as a white solid.

Synthesis of tert-Butyl acetyl(7-chloro-3-cyclopropylbenzisoxazol-5-yl)carbamate (42-7). A mixture of 789.3 mg (42-6), 808 mg Boc₂O and 38 mg DMAP in 40 mL dry DCM was stirred at room temperature for 1 h. Solvent was evaporated to give crude (42-7) as a white solid.

Synthesis of tert-Butyl (7-chloro-3-cyclopropylbenzisoxazol-5-yl)carbamate (42-8). The above crude (42-7) was dissolved in 100 mL MeOH. The solution was basified with 0.1 mL 25 wt. % NaOMe/MeOH, and then stirred at room temperature for 30 min. To the solution was added 1 g solid NH₄Cl, and the solvent was evaporated. The residue was partitioned between 300 mL 0.1 N HCl/brine and 300 mL EtOAc. The organic layer was separated, washed sequentially with 100 mL 0.1 N HCl/brine, 100 mL water, 100 mL saturated NaHCO₃ and 100 mL water, dried with MgSO₄ and evaporated. The residue was crystallized from heptane-EtOAc to give pure (42-8) as a white solid.

Synthesis of 5-[(tert-Butoxycarbonyl)amino]-7-chloro-3-cyclopropylbenzisoxazole-6-carboxylic acid (42-9). A solution of 770 mg (42-8) in 50 mL dry THF was protected under argon, and stirred with dry ice/acetone bath cooling. To the solution was added dropwise 5.9 mL 1.7 M tBuLi/pentane under vigorous stirring. After complete addition, the mixture was further stirred at −78° C. for 5 min. To the latter was added all at once 7.2 g freshly crushed dry ice under vigorous stirring. The mixture was stirred at −78° C. for 5 min, and then allowed to warm up to room temperature. The reaction mixture was partitioned between 300 mL 1 N HCl/brine and 300 mL EtOAc. The organic layer was separated, washed with 100 mL 0.1 N HCl/brine, dried with MgSO₄ and evaporated. The residue was separated by silica gel column chromatography with hexanes/EtOAc/HOAc as eluent to give (42-9) as an off-white foam.

Synthesis of methyl 5-[(tert-butoxycarbonyl)amino-7-chloro-3-cyclopropylbenzisoxazole-6-carboxylate (42-10). A solution of 815 mg (42-9) and 5 mL MeOH in 10 mL DCM was stirred with ice bath cooling. To the solution was added dropwise 2.31 mL 2 M trimethylsilyldiazomethane (TMSCHN₂) in hexanes under stirring. After complete addition, the solution was stirred at room temperature for 10 min and evaporated. The residue was dissolved in 100 mL DCM, and the solution was passed through a short silica gel column. The column was eluted with MeOH-DCM, and the combined fractions were evaporated to give (42-10) as an off-white solid.

Synthesis of methyl 5-amino-7-chloro-3-cyclopropylbenzisoxazole-6-carboxylate (42-11). A solution of 813 mg (42-10) in 10 mL DCM was stirred with ice bath cooling. To it was added dropwise 10 mL TFA with stirring. After complete addition, the mixture was stirred at room temperature for 30 min and evaporated. The residue was partitioned between 200 mL saturated NaHCO₃ and 200 mL EtOAc. The organic layer was separated, washed with water (2×50 mL), dried with MgSO₄, and evaporated to give (42-11) as a yellow oil, which solidified on standing.

Synthesis of 2-(5-Amino-7-chloro-3-cyclopropylbenzisoxazol-6-yl)propan-2-ol (II-42). A solution of 7.73 mL 3M MeMgCl/THF in 6 mL dry THF was protected under argon and stirred with ice bath cooling. To it was added dropwise a solution of 620 mg (42-11) in 50 mL dry THF under vigorous stirring. After complete addition, the mixture was allowed to warm and then stirred at room temperature for 1 h. The mixture was added carefully into 300 mL saturated aqueous NH₄Cl under stirring and ice bath cooling. The mixture was extracted with DCM (3×100 mL), dried with MgSO₄ and evaporated. The crude product was purified by silica gel column chromatography with MeOH-DCM as eluent, and then crystallized from heptane-DCM to give pure (II-42) as an off-white solid. ¹H NMR (400 MHz, CDCl₃) δ: 1.10 (m, 2H), 1.15 (m, 2H), 1.91 (s, 6H), 2.09 (m, 1H), 4.33 (br, 3H, NH₂ and OH), 6.70 (s, 1H). ¹³C NMR (100 MHz, CDCl₃) δ: 7.11, 7.25, 30.7, 77.1, 105.6, 113.7, 120.4, 132.5, 144.4, 155.4, 160.5. LC-MS: 267.1 (MH)⁺, 269.1 [(M+2)H]⁺.

### Example 10: Synthesis of II-43

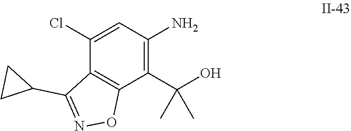 [SMILES: CC(C)(O)c1c(N)cc(Cl)c2c(C3CC3)noc12]

Synthesis of 1-(6-Amino-4-chloro-3-cyclopropyl-benzisoxazol-7-yl)ethanone (43-3). To a mixture of 636 mg (43-2) and 43 mg CuI was slowly added 8.16 mL 3 M MeMgCl/THF under stirring and ice bath cooling. The suspension was protected under argon, and heated at 70° C. in an oil bath for 15 min. The mixture was cooled to 0° C. in an ice bath. To it was added 136 mL MeOH, followed by 2.17 g solid NH₄Cl and 13.6 mL water. The mixture was warmed to room temperature with stirring to give a clear solution, which was adsorbed on silica gel, air-dried and separated by silica gel column chromatography with hexanes-EtOAc as eluent to give (43-3) as a yellow solid.

Synthesis of 2-(6-Amino-4-chloro-3-cyclopropylbenzisoxazol-7-yl)propan-2-ol (II-43). A mixture of 1.54 mL 3 M MeMgCl/THF and 5 mL dry THF was protected under argon and stirred with ice bath cooling. To it was added a solution of 387.1 mg (43-3) in 15 mL dry THF under vigorous stirring. After complete addition, the solution was further stirred at 0° C. for 20 min. To the solution was added 100 mL saturated aqueous NH₄Cl with ice bath cooling and vigorous stirring. The mixture was warmed to room temperature and extracted with DCM (3×100 mL). The combined organic layers were dried with MgSO₄ and evaporated.

The crude product was purified by silica gel column chromatography with MeOH-DCM as eluent, and crystallized from heptane-DCM to give pure (II-43) as a light tan solid. ¹H NMR (400 MHz, CDCl₃) δ: 1.12 (m, 2H), 1.18 (m, 2H), 1.78 (s, 6H), 2.17 (m, 1H), 4.86 (br, 2H, NH₂), 6.60 (s, 1H). ¹³C NMR (100 MHz, CDCl₃) δ: 8.81, 9.26, 30.1, 74.1, 112.7, 114.4, 121.8, 131.3, 143.8, 148.6, 166.1. LC-MS: 267.0 (MH)⁺, 268.9 [(M+2)H]⁺.

### Example 11: General Reaction Sequence to Prepare IV-1 and IV-2

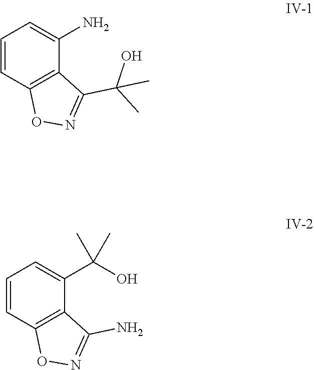

Compounds of the invention (e.g., formulae (IV-1) and (IV-2)) can be prepared as shown in Schemes 2-1 and 2-2.

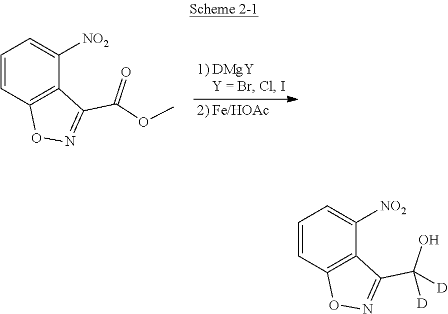 [SMILES: COC(=O)c1noc2cccc([N+](=O)[O-])c12 ; [2H]C([2H])(O)c1noc2cccc([N+](=O)[O-])c12]

Starting material may be made by methods known in the art, such as that described in Ji Z. et al., Bioorg. & Med. Chem. Let. (2012), 22, 4528.

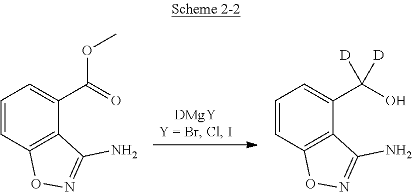

Starting material may be made by methods known in the art, such as that described in Smalley, R. K., 2002, Science of Synthesis 11:289.

### Example 12: Synthesis of NS2-SSA Conjugate

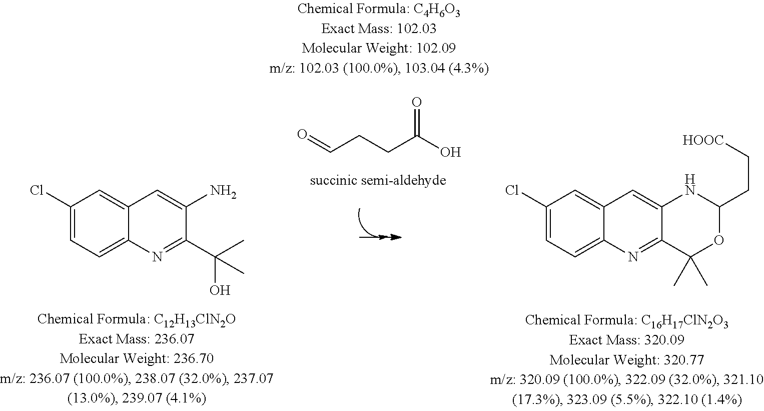 [SMILES: CC(C)(O)c1nc2ccc(Cl)cc2cc1N ; O=CCCC(=O)O ; CC1(C)OC(CCC(=O)O)Nc2cc3cc(Cl)ccc3nc21]

NS2 and succinic semi-aldehyde (SSA) solutions were added to a mixture of acetonitrile, water and hydrochloric acid and incubated for 1 h at room temperature to form the NS2-SSA conjugate. This solution was infused directly onto a Sciex 6500 for mass spectrometer optimization. Decoupling potential, 30 V; Curtain gas, 20; CAD, High; Ion Spray Voltage, 4500 V; Source temperature, 450° C.; Ion Source gas 1, 50; Ion Source gas 2, 50; entrance potential, 10 V. NS2 was quantified using the 237.0 fragment, whereas NS2-SSA was quantified using the 321.1 fragment.

### Example 13: In Vitro Assays

### LDH Cytotoxicity Assay

Primary rat cortical cultures are placed in an incubator for 24 or 48 hours and treated with various concentrations of disclosed compounds. Then 20 μL of the culture media is removed for an LDH assay as described in Bergmeyer et al., Methods of Enzymatic Analysis, 3^{rd} ed. (1983).

### ELISA Assay to Determine Amount of Circulating Cytokines

Male C57BI/6 mice are dosed with disclosed compounds 30 minutes before they are exposed to LPS (20 mg/kg). Two hours after the LPS exposure, blood is collected from the mice and an ELISA is conducted to determine the amount of circulating cytokines. Treatment with disclosed compounds leads to reduction in proinflammatory cytokines, such as IL-5 and IL-1β, IL-17, and TNF. Also, treatment with disclosed compounds results in elevated anti-inflammatory cytokines, such as IL-10. In addition, various other chemokines, such as eotaxin, IL-12, IP-10, LIF, MCP-1, MIG, MIP, and RANTES, are also decreased by treatment with disclosed compounds.

### Assay to Evaluate Efficacy in Treating Contact Dermatitis

To determine the efficacy of the disclosed compounds in treating contact dermatitis, phorbol myristate acetate (“PMA”) is applied topically (2.5 μg in 20 μL) to both the anterior and posterior portions of the right pinna of mice (N=10 per group). As a control, the left pinna receives 20 μL of ethanol (PMA excipient) to both the anterior and posterior portions. Six hours after the PMA application, both the right and left pinna thickness is determined. Measurements are determined at least twice from the same region of both ears, with care taken not to include hair or folded pinna.

### Assay to Evaluate the Efficacy in Treating Allergic Dermatitis

To measure the efficacy of the disclosed compounds in treating allergic dermatitis, oxazolone (“OXL”) is applied (1.5%, 100 μL in acetone) to the shaved abdomens of mice. Seven days later, the thickness of the pinna of the OXL treated mice is determined. Then the disclosed compounds (100 mg/kg) or the vehicle (i.e., Captisol) is administered intraperitoneally to mice followed by topical application of OXL (1%, 20 μL) 30 min later to both the anterior and posterior portions of the right pinna. As a control, the left pinna receives 20 μL of acetone (OXL excipient) to both the anterior and posterior portions. The thickness of the pinna of both ears is measured again 24 hours later. N=10 per group.

### Assay to Measure Aldehyde Trapping

To separate reaction vials is added each disclosed compound, (0.064 mmol), MDA salt (22.7% MDA, 0.064 mmol), and glyceryl trioleate (600 mg). To the mixture is added 20 wt % Capitsol in aqueous PBS (˜2.5 ml), followed by linoleic acid (600 mg). The reaction mixture is stirred vigorously at ambient temperature and monitored by LC/MS. The disclosed compounds quickly react with MDA to form MDA adducts.

### Schiff Base Confirmation

UV/VIS spectroscopy is used to monitor Schiff base condensation of RAL with the primary amine of a compound of the invention. The in vitro analysis of the Schiff base condensation product with RAL is performed for the disclosed compounds.

In the solution phase analysis, the λₘₐₓ value of both the free compound and the RAL Schiff base condensation product (RAL-SBC) are measured along with the value for tau of the RAL-SBC. As used herein, “RAL-SBC” means the Schiff base condensation product of RAL and a RAL-compound. Solution phase analysis is performed using a 100:1 mixture of compound and RAL using protocols known in the art. Several solvent systems were tested including aqueous, ethanol, octanol, and chloroform:methanol (various e.g., 2:1). The solution kinetics are measured and found to be highly dependent on solvent conditions.

Solid phase analysis of the Schiff base condensation is also performed using a 1:1 mixture of compound to RAL. The solid phase analysis is performed using protocols known in the art. The mixture is dried under nitrogen and condensation reaction occurs to completion.

Lipid phase analysis is performed using protocols known in the art and λₘₐₓ, tau (RAL-SBC vs. APE/A2PE), and competitive inhibition are measured. Liposome conditions are closer to in situ conditions.

### ERG Analysis of Dark Adaptation

Dark adaptation is the recovery of visual sensitivity following exposure to light. Dark adaptation has multiple components including both fast (neuronal) processes and a slow (photochemical) process.

Regeneration of visual pigment is related to the slow photochemical process. Dark adaptation rates are measured for several reasons. Night blindness results from a failure to dark adapt (loss of visual light sensitivity). It is possible to find a safe dose for night vision by measuring drug effects on dark adapted visual light sensitivity.

An electroretinogram (ERG) is used to measure dark adaptation under normal vs. drug conditions. ERG is the measurement of the electric field potential emitted by retinal neurons during their response to an experimentally defined light stimulus. More specifically, ERG measures retinal field potentials at the cornea after a flash of light (e.g., 50 ms). Field strengths are 102 to 103 microvolts, originating in retinal cells.

ERG is a non-invasive measurement which can be performed on either living subjects (human or animal) or a hemisected eye in solution that has been removed surgically from a living animal. ERG requires general anesthesia which slows dark adaptation and must be factored into experimental design.

In a typical ERG analysis of dark adaptation experiment, every rat is dark adapted for hours to reach a consistent state of light sensitivity. The rat is then “photo-bleached,” i.e., exposed briefly to light strong enough to transiently deplete the retina of free 11-cis-RAL (e.g., 2 min at 300 lux). The rat is then returned to dark immediately to initiate dark adaptation, i.e., recovery of light sensitivity due to regeneration of visual pigment. ERG is used to measure how quickly the rat adapts to dark and recovers light sensitivity. Specifically, a criterion response variable is defined for light sensitivity.

The ERG measurement is taken after a specific duration of post-bleach dark recovery (e.g., 30 min) determined previously by kinetic analysis. A curve fit is used to calculate value for the sensitivity variable and shows recovery with anesthesia in the same rat including dark adaptation kinetics for Y₅₀ and σ. Slower adaptation is observed with less light sensitivity where Y₅₀ reaches −4.0 and tau=22.6 min. Faster adaptation is observed with more light sensitivity where Y₅₀ reaches −5.5 and tau=9.2 min.

The same paradigm as described above is followed for dose ranging. in the ERG dose ranging protocol, compounds i.p. lowers light sensitivity of dark adapted rats in a dose dependent manner. The effect on vision decreases after 3 hours.

### NMR Analysis of RAL Reaction

NMR spectroscopy is used to monitor Schiff base condensation and ring formation of RAL with the primary amine of a compound of the invention.

### Inhibition of A2E Formation

This experiment is designed to establish proof of concept that chronic i.p. injection of a RAL-trap compound lowers the accumulation rate of A2E in wild type Sprague Dawley rats. These experiments compare the treatment efficacy of RAL-trap compounds to that of control compounds and lack of treatment.

### Materials and Methods:

The study is performed with wild type Sprague Dawley rats. Rat treatment groups include, for example, 8 rats of mixed gender per treatment condition. Each animal is treated with one of the following conditions:
Controls: (1) 13-cis retinoic acid to inhibit retinoid binding sites of visual cycle proteins as a protocol control, in that such treatment reduces the amount of free trans-RAL that is released and thereby available to form A2E, but with undesirable side effects of night blindness, and (2) a commercially available compound known clinically to modulate retinal function in humans and known experimentally to form a Schiff base adduct with free RAL, both in vitro and in vivo in animal models.
Vehicle
Compound
Untreated

The disclosed compounds are tested across a dose range including 1, 5, 15, and 50 mg/kg. Treatment is administered daily for 8 weeks by i.p. injection.

### Chemistry:

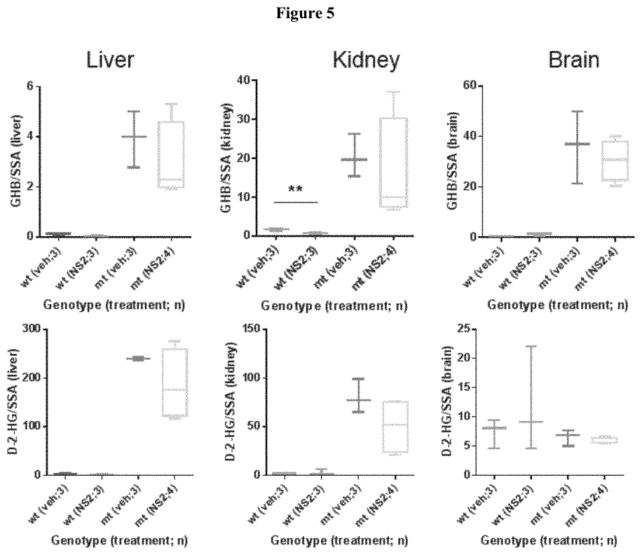

The experiments use a variety of chemistry services. For example, these experiments use commercially available compounds with analytical specification sheets to characterize the impurities. Compounds are also synthesized. Compounds are prepared in quantities sufficient for the required dosing. Formulations of the compound are suitable for use in initial animal safety studies involving intraperitoneal (i.p.) injection. The following three attributes of the Schiff base reaction product of trans-RAL with compounds of the invention are determined:
stability with respect to reaction rates
absorption properties, specifically uv-vis absorption maxima and extinction coefficients (see e.g., FIG. 5 in Rapp and Basinger, Vision Res. 22:1097, 1982) or NMR spectral analysis of reaction kinetics
log P and log D solubility values e.g. calculated

### Biology and Biochemistry:

The experiments described herein use a variety of biology and biochemistry services. A “no effect level” (NOEL) dose of compounds of the invention for daily treatment with an eye drop formation is established, e.g., in the rabbit with an ocular irritation protocol and in the rodent with ERG measurement of dark adaptation in visual responses to light stimulation. After treatment and before eye enucleation, the following non-invasive assays are performed in animals, e.g., rabbits:
RPE and photoreceptor cell degeneration, as evident by fundus photography (Karan, et al., 2005, Proc Natl Acad Sci USA. 102(11):4164-9)
Extracellular drusen and intracellular lipofuscin as measured by fundus fluorescent photography (Karan et al. 2005)

Light responses are characterized by ERG (Weng, et al., 1999, Cell 98:13). Intracellular A2E concentration of retinal RPE cell extracts is measured in all treated animals upon the conclusion of the treatment protocol using an analytical method such as those described by Karan et al., 2005, Proc Natl Acad Sci USA. 102(11):4164-9; Radu et al., 2003, Proc Natl Acad Sci USA. 100(8):4742-7; and Parish et al., 1998, Proc Natl Acad Sci USA. 95(25):14609-13. For example, in a sample of treated animals, one eye is assayed, and the other eye is saved for histology analysis (as described below). In the remaining animals, both eyes are assayed separately for A2E formation.

In the post-treatment eyes set aside for histology (as described above), the morphology of retinal and RPE tissue is assessed with light microscopy histology techniques (Karan et al., supra, with the exception that electron microscopy is not used in the experiments described herein).

The safety of the treatment regimen is assessed for example using a combination of:
Daily documented observation of animal behavior and feeding habits throughout the treatment period
Visual performance as measured by ERG at the end of the treatment period
Ocular histology at the end of the treatment.

### Example 14: Preclinical Testing of NS2 in a Mouse Model of SSADH Deficiency

Since SSADH is an aldehyde-metabolizing enzyme, and since its substrate, SSA, is known to accumulate in SSADH deficiency and is hypothesized to lead to accumulation of further downstream metabolites, it was hypothesized that treatment of SSADH null mice with NS2 could lead to production of the NS2-SSA adduct and modulate various metabolites in target organs, as well as lead to improvement in the phenotype of the model.

The objective of the current experiment was to assess initial pharmacokinetics of NS2 and measure and compare various SSA metabolites in SSADH null mice and their wild type counterparts eight hours after a single intraperitoneal (i.p.) dose of NS2 or vehicle.

### Summary:

Initially a pharmacokinetic study was conducted to demonstrate that NS2-SSA adducts indeed can form in vivo. Wild type mice were injected with one i.p. dose of either NS2 (10 mg/kg) or vehicle (DMSO, 7.8±1.4%; diluted to a total volume of 100 μL in PBS). Mice were 41-46 days old on the day of treatment, and groups (n=3) were balanced for age, gender and starting weight (18±3 g). NS2 was tolerated in these mice, in this 24-hour single dose study, which primarily targeted initial NS2 pharmacokinetics and in vivo formation of NS2-SSA adducts. The results of this study informed the design of a subsequent 8-hour single dose study to measure additional biochemical outcomes (GHB and related metabolites) in both SSADH deficient mice and wild type littermates.

Loss of SSADH in mice results in a severe presentation of the human disease, including failure to gain weight after day 15, small size, absence of fat mass, and neurological impairment. They are characterized by a critical period between days 16-22 that includes generalized tonic-clonic seizures. There is 100% mortality by 3-4 weeks of age (varies by colony). In these mice, levels of brain GABA are 2-3 times higher and brain GHB is 20-60 times higher than in wild type mice. For additional information on SSADH knock out mice, see Hogema et al., 2001, Nat Genet. 29:212-16.

### Experimental Design:

Mouse Model: B6.129-Aldh5a1^{tm1Kmg}/J. Mice homozygous for the Aldh5a1 knockout exhibit reduced body weight, ataxia, seizures, gliosis of the hippocampus, and eventual status epilepticus. From 19-26 days of age, repetitive tonic-clonic seizures results in more than 95% mortality. Biochemical assays shows complete ablation of the endogenous enzymatic activity in the brains, livers, hearts, and kidneys of homozygous mutant mice. Homozygotes have increased levels of GHB and GABA in liver and brain tissues, as well as in urine. The phenotype can be rescued to varying degrees utilizing a number of pharmacotherapeutic and gene therapeutic approaches. Although heterozygous mice have approximately 50% of the endogenous enzyme activity compared to wild type mice, they are viable and fertile. Mice with this targeted mutation may be useful in studying succinate semialdehyde dehydrogenase (SSADH) deficiency and to explore the effect of GABA and GHB accumulation on central nervous system development and function.

Test article was NS2 API powder Batch BR-NS2-11-01. Material was stored at −80° C. Material was weighed out and dissolved in 100% DMSO to create a stock solution of 25 mg/ml, with further dilution in PBS as necessary to maintain a constant dose volume, based upon body weight. Final NS2 dosing solution was vigorously shaken and vortexed, but not filtered. Solution was handled using aseptic techniques. DMSO was used as the vehicle and was obtained from Sigma-Aldrich.

SSADH null mice and their wild type littermates were injected with one i.p. dose of either NS2 (10 mg/kg) or vehicle (DMSO, diluted to a total volume of 50 μL in PBS; 5.9±2.3% DMSO). Mice were 22-23 days old on the day of treatment, and groups (n=3) were balanced for age and gender. NS2 was well-tolerated in these mice in this 8-hour study, which primarily targeted initial NS2 pharmacokinetics and measurement of SSA metabolites. Future studies in this model will encompass a dose-finding paradigm to ensure adequate target exposure; dose earlier in life; and increase group sizes.

### Group Assignments and Treatments:

A. Preliminary Single Dose, IP Pharmacokinetics in Wild Type Mice

A preliminary assessment of single dose, i.p. pharmacokinetics (PK) was conducted in wild type C57Bl6 mice. Mice were 41-46 days old at the time of dosing. Mice were administered NS2 at 10 mg/kg and samples for analysis of NS2 and NS2-SSA adducts were taken at (baseline, 0.5, 1, 1.5, 3, 7, 12, 22 hours) after dosing. Three mice per timepoint were used for pharmacokinetic analysis. The study design is outlined in Table 6 below.

TABLE 6
Design of preliminary single dose, i.p.
pharmacokinetics study in wild type mice

| | | NS2 | Dose volume | | |
|---|---|---|---|---|---|
| Group | Timepoint | (mg/kg) | (mL/kg) | # Mice | RoA |
| 1 | 0, | 0 | 0.4 | 3 | i.p. |
| | baseline | | | | |
| 2 | 0.5 | 100 | 0.5 | 3 | i.p. |
| 3 | 1 | 100 | 0.4 | 3 | i.p. |
| 4 | 1.5 | 100 | 0.4 | 3 | i.p. |
| 5 | 3 | 100 | 0.4 | 3 | i.p. |
| 6 | 7 | 100 | 0.4 | 3 | i.p. |
| 7 | 12 | 100 | 0.4 | 3 | i.p. |
| 8 | 22 | 100 | 0.4 | 3 | i.p. |

RoA = Route of Administration

B. Effects of NS2 on Selected Metabolites in Wild Type and SSADH Null Mice

Wild type and SSADH null mice were administered single intraperitoneal (i.p.) doses of NS2 (10 mg/kg) or vehicle (0.4 μL DMSO/g bodyweight, 100% in PBS). Eight hours after dosing, animals were sacrificed and tissues (liver, kidney, brain and blood) were harvested for analysis of NS2 concentrations and metabolite concentrations. The study design is shown in Table 7.

TABLE 7
Design of eight-hour NS2 treatment study

| | Mice | | | | | |
|---|---|---|---|---|---|---|
| | (number | Number | | NS2 | Dose | |
| | intended | actually | | Dose | Volume | |
| Group | to treat) | treated | Treatment | (mg/kg) | (2 mL/kg) | RoA |
| Group 1 | 3-4 | 3 | DMSO | 0 | 50 microliter | i.p. |
| | wild type | | | | | |
| Group 2 | 3-4 | 4 | NS2 | 10 | 50 microliter | i.p. |
| | wild type | | | | | |
| Group 3 | 3-4 | 3 | DMSO | 0 | 50 microliter | i.p. |
| | SSADH | | | | | |
| | null mice | | | | | |
| Group 4 | n = 3-4 | 4 | NS2 | 10 | 50 microliter | i.p. |
| | SSADH | | | | | |
| | null mice | | | | | |

RoA = Route of Administration

The design of the study was as follows:

Treatment groups were balanced for date of birth, gender, and weight.
null mice were generated by crossing mice heterozygous for SSADH deficiency. Expected number of null progeny is 1 in 4. Seven SSADH null mice were generated from this breeding, of which three were assigned to group 3 and four were assigned to group 4. SSADH status was determined by genotyping from tail snips on post-natal day 9 or 10.
Treatment groups were randomly assigned prior to dosing.
Dosing order was balanced across treatment groups and maintained throughout study.
Dosing route of administration was intraperitoneal (i.p.) injection, using a 25 gauge needle.
Vehicle or NS2 was administered systemically by i.p. injection.

### Test Article Preparation and Dosing:

On the day of dosing, NS2 and vehicle were brought to room temperature. Once at room temperature, a working solution of NS2 was made by dissolving 25 mg NS2 in 1 mL 100% DMSO to yield a 25 mg/mL working solution. This working solution was prepared at room temperature, using aseptic technique in the animal dosing suite, and was used within one hour of preparation.
Dosing volume was ˜0.4 μL/g body weight for both mutant and wild-type subjects (note: the average body weight for the SSADH null mice is ˜4.9±0.9 g, and that of age-matched wild-type littermate is 10.2±0.9 g). DMSO total dose is weight normalized.
Leftover working solutions were discarded.

### Animal Monitoring:

Overall health was assessed cage-side, for all mice until sacrifice:
Standard diet and water were available ad libitum.

### Study Termination:

Animals were sacrificed 8 hours after NS2 or vehicle administration.
   Animals were euthanized with carbon dioxide administration (1-2 min) followed by cervical dislocation.
Liver, kidney, and brain were collected. Organs were snap frozen in liquid nitrogen for biochemical analysis and stored at −80° C.
   A terminal cardiac blood sample was obtained in a standard microtainer for serum collection. Serum was prepared by centrifugation for 10 minutes at 1,000 rpm (2500×g) and stored at −80° C. until analyses.

### Methods:

### Genotyping:

Genotyping was performed as described, for example, in Hogema et al., 2001, Nat Genet 29:212-216.

### Tissue Homogenization:

For liver, ˜100 mg of frozen tissue was biopsied using a clean surgical razor and weighed 5-fold volume to weight cold phosphate buffered saline (PBS, pH=7.4) was added and tissues were homogenized with a mechanical homogenizer. One kidney and one half of the brain (left) was weighted and prepared in the same manner (weights approximated 100 mg each).

### NS2 Assay and NS2-SSA Adduct Assay:

Homogenates (100 μL) were protein-precipitated with cold acetonitrile containing 0.1% formic acid (900 μL). Serum samples (25 μL) were protein-precipitated with 425 μL of cold acetonitrile containing 0.1% formic acid. Samples were centrifuged at 2,500×g then supernatant was transferred to a clean tube and dried under a constant heated flow of nitrogen (50° C.). Samples were reconstituted in 100 μL of mobile phase A (water with 0.1% formic acid, LC-MS/MS grade reagents). Calibration standards for NS2 were prepared by spiking known concentrations into blank sera or tissue homogenates. In situ studies were utilized to obtain optimal fragmentation data for both NS2 and NS2-SSA, which were ultimately quantified via multiple reaction monitoring (MRM) using 237.0/218.9 m/z (NS2) and 321.1/167.9 m/z (NS2-SSA).

Samples (3 μL) were injected onto a Kinetix PFP UPLC Column (2.1×50 mm) chromatographic separation was achieved with a gradient method comprised, initially of 95% Mobile phase A and 5% Mobile phase B (methanol with 0.1% formic acid), this was held for 0.5 minutes then increased linearly to 95% Mobile phase B over 2.2 minutes, held constant for 0.5 minutes, then returned to initial conditions over 6 seconds and maintained at initial conditions for a total run time of 5 minutes. Eluent was direct to an API Sciex 6500 mass spectrometer operated in multiple reaction monitoring mode using 237.0/218.9 m/z for NS2 and 321.1/167.9 m/z (NS2-SSA).

### SSA, GHB, D-2-HG Assays:

Plasma and tissue homogenates were shipped to the laboratory of Professor Gajja Salomons (VU Medical Center, Amsterdam, the Netherlands) on May 11, 2015. SSA, GHB and D2HG levels were assayed in the Salomons laboratory using the following published methods: 1) “Stable isotope dilution analysis of 4-hydroxybutyric acid: an accurate method for quantification in physiological fluids and the prenatal diagnosis of 4-hydroxybutyric aciduria,” Gibson et al., 1990, Biomed Environ Mass Spectrom. 19(2):89-93; 2) “Stable-isotope dilution analysis of D- and L-2-hydroxyglutaric acid: application to the detection and prenatal diagnosis of D- and L-2-hydroxyglutaric acidemias,” Gibson et al., 1993, Pediatr Res. 34(3):277-80; 3) “Metabolism of gamma-hydroxybutyrate to d-2-hydroxyglutarate in mammals: further evidence for d-2-hydroxyglutarate transhydrogenase,” Struys et al., 2006, Metabolism 55(3):353-8; 4) “Determination of the GABA analogue succinic semialdehyde in urine and cerebrospinal fluid by dinitrophenylhydrazine derivatization and liquid chromatography-tandem mass spectrometry: application to SSADH deficiency,” Struys et al., 2005, J Inherit Metab Dis. 28(6):913-20.

### Tissue Analysis:

The scientist conducting tissue analysis was blinded to treatment ID. This was achieved by omitting treatment group from the dissection sheet (for the samples shipped to the VU Medical Center), and the use of personnel at Washington State University who had not had access to data records for the in-life phase, to conduct the tissue analysis. Individuals plotting data and performing statistical analyses were not blinded to genotype and treatment.

### Results:

No animals died during the 24-hour (0.5, 1, 1.5, 3, 7, 12, 22 h), single dose PK study or the 8-hour treatment study, nor was there any indication of toxicity to the animals.

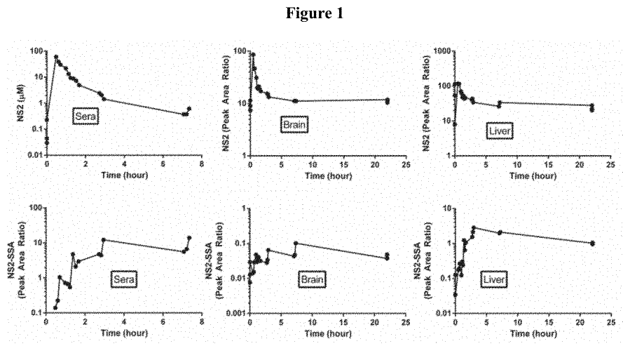

In the preliminary PK study, subjects were administered a single dose of NS2 (10 mg/kg; a dosing paradigm comparable to that used in the 8-hour metabolite study) for pharmacokinetic analysis of NS2 (FIG. 1) and measurement of NS2-SSA adduct formation. These studies were performed only in wild-type C57Bl6 mice (n=21). Animals were administered 10 mg/kg NS2 as an i.p. bolus and harvested at the denoted time points (methods followed above protocols). NS2 was prepared in DMSO (25 mg/mL), diluted in PBS, and administered in a volume of 100 microliters. The mice ranged in age from 41-46 days of age.

The amounts of NS2 in brain and liver, and NS2-SSA adduct in serum, brain and liver, are expressed as the analyte signal normalized to the internal standard (PAR, or peak area ratio) because an authentic standard for the NS2-SSA adduct was not available; serum NS2 is expressed as micromole/liter.

The data shown in FIG. 1 indicate first-order pharmacokinetics for NS2. The data show that NS2 rapidly (0.5 h) reaches peak serum concentration (43.1±15.4 μM) after i.p. administration. Peak concentrations in the brain and liver were similar to that observed in serum (52.4±22.9 and 116±3.1, respectively) and were also reached. NS2 levels in serum declined to less than the LLOQ (LLOQ 50 nM)) by 24 hours. NS2-SSA adduct in serum, brain and liver was sustained at nearly the maximal levels for the duration of the 24-hour study.

Analysis of NS2-SSA adducts revealed a time-dependent increase in the formation of NS2-SSA adducts in serum, brain and liver. Following NS2 dosing, maximum levels of the NS2-SSA adduct were observed at 3 hours in serum, 8 hours in brain and 3 hours in liver.

In the metabolite study, both wild type and SSADH null mice were administered a single i.p dose of NS2 (10 mg/kg). Based on the concentrations of NS2 and NS2-SSA adducts observed in serum, liver and brain during the course of the preliminary PK study, the time point of 8 hours post-dose was selected for tissue harvest.

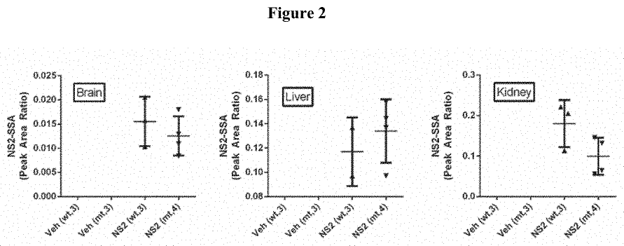

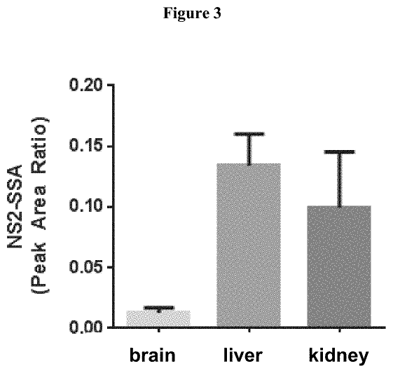

As shown in FIG. 2, NS2-SSA adduct was found in wild-type tissues and in mutant animals. There was no significant difference in any measurement between the wild type and null mice, although the level of adduct tended to be higher in liver from null mice. FIG. 3 shows an alternate view of brain, liver, and kidney levels of NS2-SSA adduct after NS2 administration as a single dose to SSADH knock-out mice.

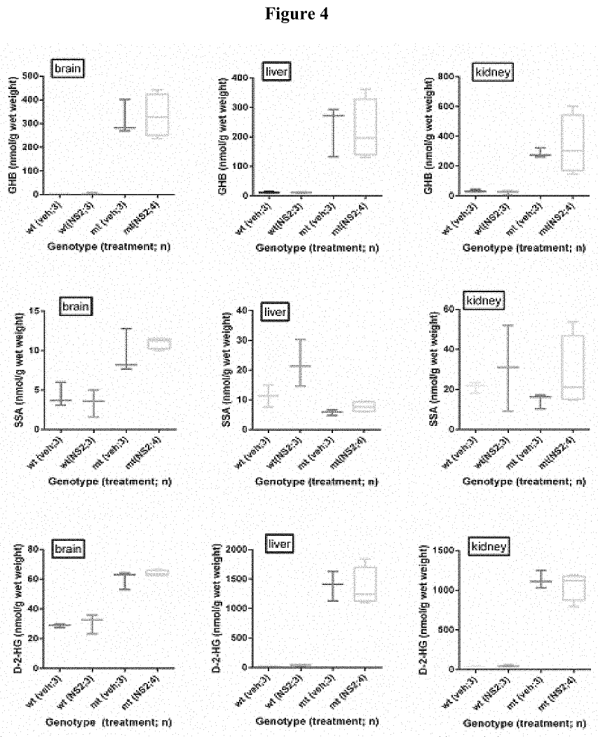

Tissues from animals in the metabolite study were analyzed for GHB, SSA and D-2-HG (see FIG. 4 below) in the laboratory of Professor Gajja Salomons (VU Medical Center, Amsterdam). In the SSADH null mice treated with NS2, there was a tendency for decrease of GHB and D-2-HG in liver, but there were no statistically significant changes in the levels of these metabolites.

FIG. 5 shows the GHB/SSA and D-2-HG/SSA levels of SSADH null mice (22-23 days old) who received one dose of 10 mg/kg NS2 or vehicle (IP) compared with those of wild type mice. Brain, liver and kidney were harvested 8 hours following treatment (statistical analysis: student's t test (**p<0.01)).

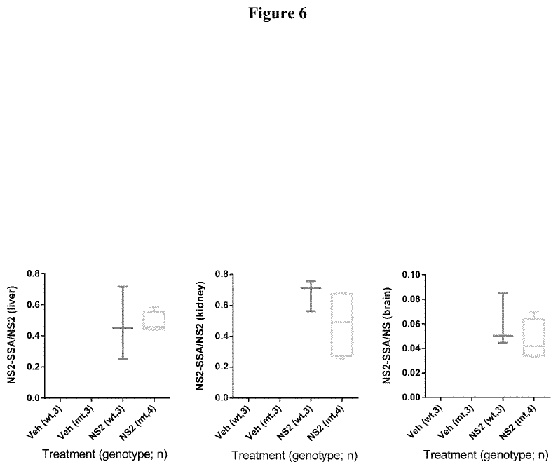

FIG. 6 shows levels of NS2-SSA adduct in tissues from wild type and SSADH null mice treated with vehicle or NS2.

### Discussion:

This study was conducted in two stages. First, a preliminary, single dose, i.p. pharmacokinetic study was performed in wild type mice to assess the pharmacokinetic profile of NS2 and the rate and extent of NS2-SSA adduct formation. These data were used to choose the timepoint for tissue analyses in the second study, which studied the effects of NS2 on the formation of selected metabolites, including SSA, in wild type and SSADH null mice.

In the preliminary pharmacokinetic study, NS2 showed a typical pharmacokinetic profile in serum, demonstrating first-order elimination kinetics of NS2 following a single dose. As brain and liver are target organs in SSADH-deficient mice, NS2 was also measured in those tissues and indicated first-order kinetics in all tissues, with good brain penetration. NS2 in brain and liver rapidly reached maximal concentrations followed by a drop to a level that was sustained for the duration of the 24-hour study. NS2-SSA adduct formation was also measured, although since an authentic calibration standard is not available, the data can only be considered semi-quantitative. NS2-SSA adduct was detected after NS2 administration, showing that even in wild type mice with presumably adequate SSADH activity, a pool of free SSA exists which is available for covalent adduction to NS2. The timing of peak adduct formation in the three tissues appeared to lag slightly behind the timing of peak NS2 concentrations in the tissues. Sustained levels of adduct observed were observed for the duration of the 24-hour study. This could reflect a constant, steady-state production of adduct, stability and slower clearance of already-formed adduct in the tissue, or both. Levels of the NS2-SSA adduct were highest in liver and serum, and lower in brain. The lower levels observed in brain cannot be attributed to hindered access of NS2 to the brain, because approximately equivalent levels of NS2 were observed in serum, brain and liver. Alternatively, if lower levels of SSA are observed in wild type mouse brain, compared to liver and serum, one might expect to see lower levels of the adduct in brain, relative to serum and liver. However, because SSA was not measured independently in this study, it is not immediately clear why adduct levels were lower in brain.

Because NS2 levels were still high at eight hours post-dose and NS2-adduct levels had peaked by eight hours post-dose, eight hours was chosen as the harvest time point for the metabolite study. In this 8-hour metabolite study, mice deficient in SSADH were used to determine whether administration of a single dose of NS2 modulates levels of GHB, SSA and D-2-HG (D-2-hydroxyglutaric acid). It is believed that NS2 may be able to target and modulate levels of SSA, and accordingly GHB, D-2-HG and even DHHA (4,5-dihydroxyhexanoic acid; not measured in this study), which are hypothesized to be generated from SSA. Simultaneously, qualitative amounts of NS2-SSA adduct were estimated in the same tissues.

As in the preliminary PK study, NS2-SSA adducts were detected in the brain and liver of wild type mice. They were also detected in a third target organ, the kidneys. Similar levels were detected in these three target organs of the SSADH null mice.

There were clearly higher levels of SSA in the brains of SSADH null mice compared to their wild type counterparts, although a comparison of SSA levels in liver and kidney of wild type and SSADH null mice did not yield a clear relationship. In contrast, high levels of both GHB and D-2-HG were observed in the brains, livers and kidneys of SSADH null mice, relative to wild type littermates, as expected.

The observed tendency of NS2-treated SSADH null mice to have lower levels of both GHB and D-2-HG in the liver, while not statistically significant (perhaps due to very small group sizes), represents an intriguing first glimpse into the potential for NS2, via adduction of excess free SSA, to reduce levels of metabolites that are hypothesized to play a role in the pathology of SSADH deficiency. These data, coupled with the complexity of the metabolic pathways involved, support the further study of NS2 in SSADH.

### Conclusions:

It is concluded that NS2 can rapidly enter the peripheral circulation after i.p. administration, and that it can rapidly penetrate the brain and liver. NS2 was shown to conjugate with SSA in vivo, in both wild type and SSADH null mice in known target organs. The initial data described here, suggesting a possible reduction of GHB and D-2-HG in liver mediated by NS2 after only a single administration of drug, support the further study of NS2 in SSADH. Future studies in this model are intended to encompass a dose-finding phase, and repeat dosing, to ensure adequate target exposure, and include larger group size to ensure proper interpretability of results.

### Example 15: Inhibition of Fibroblast Activation to the Myofibroblast Phenotype Using NS2

This study examined the effects of NS2 on a model system of fibrosis, the in vitro activation of quiescent fibroblasts to the activated myofibroblast phenotype. It is shown here that NS2 limits fibroblast activation to the myofibroblast phenotype. Examination of the pathways involved in this inhibition suggests that NS2 treatment of cardiac fibroblasts limited the translocation of NFκB to the nuclei, a key step in the inflammatory cascade which leads to fibroblast activation and subsequent fibrosis. These data suggest that use of NS2 to limit fibroblast activation in injured tissues may help limit fibrosis and scarring.

### Methods:

Isolation of neonatal fibroblast. Thirty hearts from neonatal rats were minced and digested (Neonatal Cardiomyocyte Isolation System, LK003303, Worthington). After tissue digestion and cell dissociation, cell suspensions were plated into 35 mm dishes or onto coverslips in wells of 24-well plates. Serum-free DMEM was added to the cell suspensions and the cells allowed to adhere for 2 hrs. After two hours, the cell suspension was removed and the adherent cells were fed with DMEM containing 10% fetal bovine serum (FBS). Cells were maintained in DMEM+10% FBS for 24 hours before treatments. Treatment duration was 24 hours.

Dosing. Cell samples were divided in to 2 groups, stimulated with H₂O₂ or not stimulated with H₂O₂. Each group had four test conditions (control, 10 uM NS2, 100 uM NS2 and 1 mM NS2) in DMEM. The cells that were treated with H₂O₂ contained a final concentration of 0.001% in the wells. Drug was dissolved in 9.5% Captisol®, to a stock concentration of 5 mg/ml. Final concentration of Captisol® in the 1 mM NS2 and control wells was 0.95%. Final concentrations of Captisol® in the 100 uM and 10 uM NS2 wells was 0.095% and 0.0095%, respectively. Cells were treated for 24 hours then either fixed for immunostaining or collected as lysates for Western Blot analysis.

Immunostaining. After 24 hours of treatment, cells on cover slips were rinsed twice with PBS, fixed in 1% paraformaldehyde for 10 minutes then rinsed in PBS for immunostaining. Cells were permeabilized for 18 minutes in 0.1% Triton-X 100 in PBS, then rinsed three times in PBS for 15 minutes each. Following the rinses, cover slips were incubated in Image-IT FX Signal Enhancer (Invitrogen) to reduce background intensities in images. Subsequently, cells were washed three times 15 min with PBS, followed by one hour of blocking in 10% Normal Goat Serum Cells and 0.05% Triton-X 100, and then incubated overnight in 4° C. with primary antibodies diluted in PBS with 2% Normal Goat Serum and 0.05% Triton-X 100. Antibodies were applied at the following concentrations: Alpha-Smooth Muscle Actin 1:200 (V5228, Sigma-Aldrich), Vimentin 1:200 (V6630, Sigma-Aldrich) and NFκB 1:200 (C-20, Santa Cruz Biotech). Following overnight incubation, cover slips were brought to room temperature over 10 minutes and then rinsed in PBS for 15 min. Following 1 hour of incubation in the dark with secondary antibodies (1:100 in PBS, A-21127 and A-21136, Life Technologies), cover slips were rinsed 3 times for 15 min in PBS, incubated in the dark with DAPI (Invitrogen, 1:600) for 10 minutes, and then rinsed for 3 times in PBS. Cover slips were then mounted upside down on glass slides and examined using a Leica SP8 confocal microscope equipped with a 10× air lens. Images were taken as described and split into channels. Each channel was reviewed by eye to determine if NFκB was in the nucleus or in the cytosol.

Western Blot. Whole cell lysates were used for Western blotting; nuclear and cytosolic fractions were not prepared. DMEM from cells plated in 35 mm plates was removed, the cells rinsed quickly in PBS, then incubated in 500 ul cold RIPA buffer for 20-30 minutes at 4° C. under gentle rocking. Following RIPA incubation, cells were scraped from the dish using cell scrapers and frozen at −80° C. overnight to increase cell lysing. Once thawed, cells were sonicated for 1 minute at 80% maximal power (Biologics Model 150 B/T). Samples were centrifuged at 13K for 10 min at 4° C. then supernatant was separated from pellet for analysis by gel electrophoresis. BCA protein analysis was run to determine total protein, samples normalized to 0.2 ug/uL, and then loaded onto Novex 8% Bolt gels. Gels were run for 35 min at 200 V in MOPS buffer or until the lower molecular weight band reached the bottom of the gel. Protein was then transferred to Hybond 0.45 uM Nitrocellulose (GE Healthcare) for 1 hour. Blots were blocked for 1 hr at room temperature in 5% BSA/TBSt. Subsequently, we incubated blots with primary antibodies as described above for immunolabeling (Vimentin, 1:20K; α-SMA, 1:500; Vinculin, 1:60K, NFκB 1:200 and 1L-1β 1:200 (Santa Cruz Biotech) for 72 hours in blocking solution at 4° C.). Blots were washed three times 15 min in TBSt. Secondary antibody (AB97040, Abcam, 1:30K) in TBst was applied for 1 hr at room temperature. Blots were exposed to Advansta WesternBright™ ECL reagent (E-1119-50, Bioexpress). Blots were developed digitally using Bio-Rad Chemidoc system.

Statistical analyses. Student t-test (two-tailed); significance was assessed at p<0.05 and p<0.01

### Results:

### NS2 Inhibits Activation of Fibroblasts to the Myofibroblast Phenotype

Immunohistochemistry.

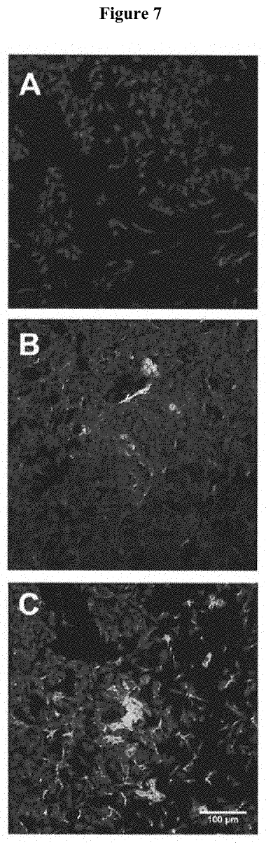

Fibroblasts in culture are known to proliferate and transform into the myofibroblast phenotype over approximately 24 hrs, regardless of stimulation with any noxious substance. Treatment of fibroblasts with H₂O₂ is known to increase the rate of this transformation. The activation of cardiac fibroblasts, unstimulated or H₂O₂-stimulated, were examined using Vimentin (Red) as a marker for fibroblasts and alpha-smooth muscle actin (α-SMA; Green) as a marker for activated myofibroblasts (FIG. 7). At plating, fibroblasts are small rounded cells with vimentin positive cytoplasms but little to no α-SMA detectable in immunostaining (FIG. 7A). Following 24 hours of incubation in media with vehicle alone, the unstimulated cells appear more flattened and have a number of filopodia, indicative of a motile cell type. Additionally, the cells spontaneously begin to convert into α-SMA positive cells indicative of activation to the myofibroblast phenotype (FIG. 7B). Cells stimulated with H₂O₂ also showed expression of α-SMA after 24 hrs in culture (FIG. 7C).

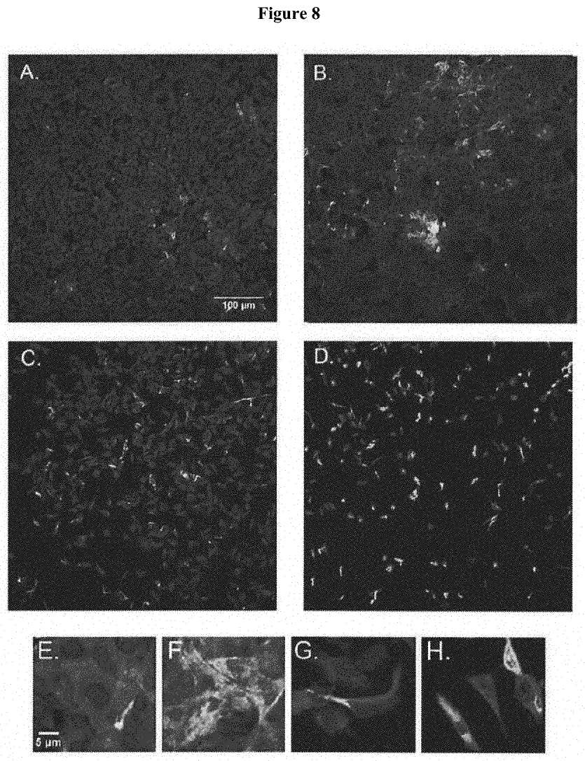

To determine if NS2 treatment could limit transformation of fibroblasts to myofibroblasts, cultured cells were treated with 10 μM, 100 μM or 1 mM NS2 and compared to cells untreated but incubated in vehicle alone (FIG. 8). Untreated cells show the presence of α-SMA 24 hrs after the cells were plated (FIG. 8A). Treatment of these cells with 10 μM NS2 appeared to have little effect on α-SMA production (FIG. 8B). In contrast, treatment with 100 μM NS2 inhibited the production of α-SMA while not limiting proliferation of cells (FIG. 8C). Increasing the levels of NS2 to 1 mM also appeared to limit the production of α-SMA but also caused the cells either to not proliferate or to undergo cell death. The few remaining cells appeared to be non-activated fibroblast phenotype. Higher magnification images indicates that the cell shape of activated myofibroblasts is flattened and has multiple points of contact (FIG. 8E), which does not change with 10 μM NS2 treatment (FIG. 8F). Increasing NS2 to 100 μM led to cells with a morphology more like that of a non-activated fibroblast (see FIG. 7) than an activated myofibroblast (FIG. 8G). Images of cells treated with 1 mM NS2 (FIG. 8H) showed fewer cells than were observed in wells of untreated cells or in wells containing cells that had been treated with vehicle (0.95% Captisol6), 10 μM or 100 μM NS2. Whether cells did not proliferate or died was not determined.

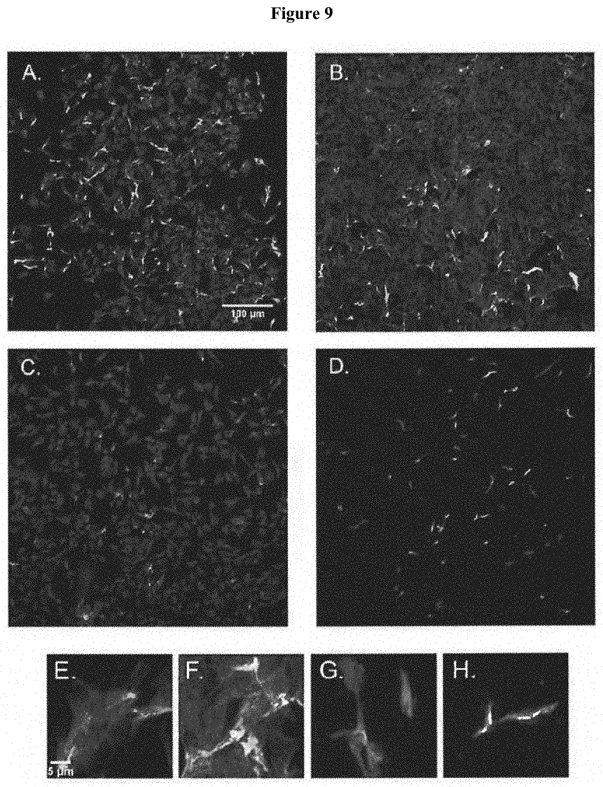

Stimulation of cardiac fibroblasts with H₂O₂ showed a very similar result (FIG. 9). Cells stimulated with H₂O₂ but not treated with NS2 show strong activation of α-SMA (FIG. 9A). As with the non-stimulated cells, cells treated with only 10 μM NS2 showed little effect on the production of α-SMA or the change of morphology consistent with the myofibroblast phenotype (FIG. 9B). Treatment of H₂O₂ stimulated cardiac fibroblasts with 100 μM NS2 led to clear inhibition of α-SMA production (FIG. 9C). After treatment with 1 mM NS2, the cells (stimulated with H₂O₂, or not) showed a morphology of a non-activated fibroblast, although there was some α-SMA observed in these cells (FIG. 9D). While one possible reason for this result is that 1 mM NS2 leads to cellular injury that is unrelated to normal fibroblast activation, no experiments were done to test this hypothesis or to suggest another reason for this. As with the non-H₂O₂-stimulated cells, NS2 treatment limited morphological changes associated with activation (FIG. 9E —no NS2; FIG. 9F—10 μM NS2; FIG. 9G—100 μM NS2; and FIG. 9H—1 mM NS2).

Western Blot of α-SMA.

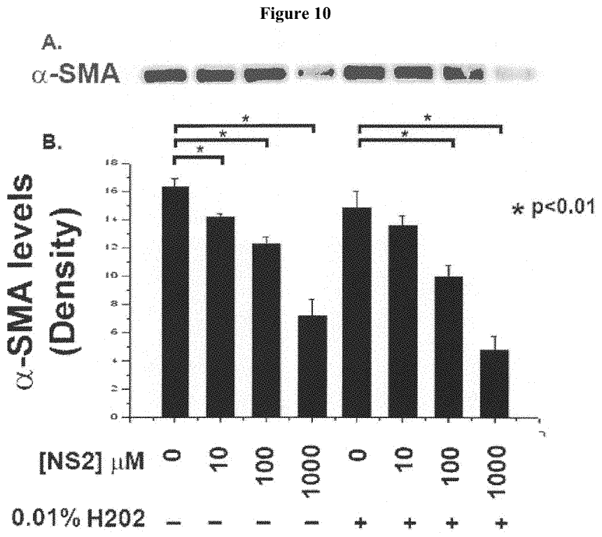

Cardiac fibroblasts were plated on 35 mm dishes for collection of cell lysates for Western Blot analysis. Plates were treated in a manner identical to the cells plated for immunostaining, that is to say cells were divided into two groups (unstimulated or H₂O₂ stimulated) then treated with either 10 μM, 100 μM or 1 mM NS2. Blots were stained for α-SMA (FIG. 10). Blots were also counterstained for GAPDH, vinculin or actinin in an attempt to find a housekeeping protein to insure normalization of the blots for analysis. Unfortunately, all the housekeeping proteins examined were altered either by the culture conditions, by the presence of NS2, or by both. All samples were assayed for protein levels and equal protein was loaded in 0, 10 μM, 100 μM NS2 while only half as much protein was loaded in the 1 mM NS2 samples, due to the low protein amounts found in these samples. The low amount of protein in the 1 mM treated cells makes the data suspect but it is included in the analysis for completeness. In non-stimulated cardiac fibroblasts, NS2 treatment led to significant decreases in α-SMA as compared to control (FIG. 10B). In H₂O₂ stimulated cardiac fibroblasts, there was no significant change with the 10 μM NS2 but increasing the dose of NS2 led to further α-SMA decreases, which were significant (p<0.01). Due to low levels of remaining cells in the 1 mM NS2 treated dishes, the data on α-SMA is not likely valid, although based on the appearance of the cells in immunostaining, it would likely be upheld if further Western Blots were done with more cells. The samples analyzed in the Western Blot analysis of FIG. 10A is as follows: Lane 1—Vehicle control; Lane 2—unstimulated treated with 10 μM NS2; Lane 3—unstimulated treated with 100 μM NS2; Lane 4—unstimulated treated with 1 mM NS2; Lane 5—H₂O₂ stimulated Vehicle control; Lane 6—H₂O₂ stimulated treated with 10 μM NS2; Lane 7—H₂O₂ stimulated treated with 100 μM NS2; Lane 8—H₂O₂ stimulated treated with 1 mM NS2.

Effect of NS2 on NFκB Translocation to the Nucleus.

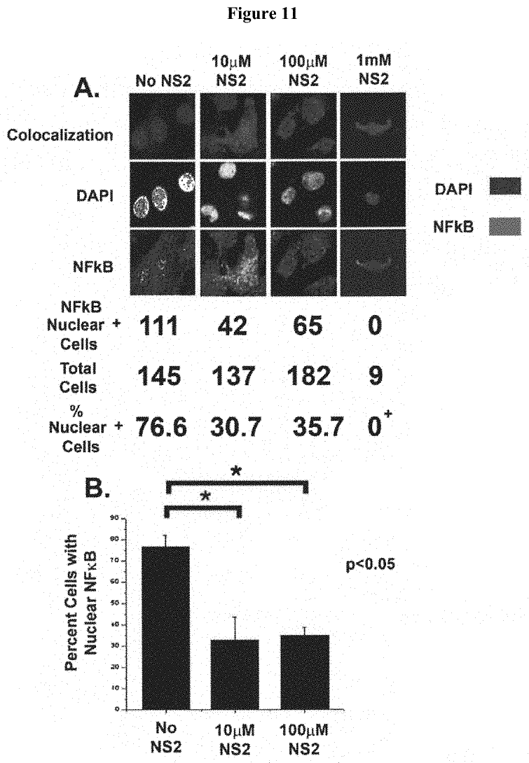

Activation of the inflammasome in cells is triggered by a number of stimuli, but all upstream pathways converge on NFκB, leading to translocation of NFκB to the cell nuclei where it is involved in activation of pro-inflammatory genes. To determine if NS2 blocked NFκB translocation, we examined cultured fibroblasts treated with NS2 and looked for localization of NFκB in the nuclei (FIG. 11A). Cell cultured for 24 hours (not stimulated with H₂O₂) showed high levels of NFκB in the nuclei of the majority of the cells (76.6%). Treatment with NS2 significantly decreased NFκB localization in cellular nuclei to 30.7% in 10 μM NS2-treated cells and 35.7% in 100 μM NS2-treated cells. There were no cells in the 1 mM NS2 treatment group that had nuclear NFκB but again, there were very few cells at all in these samples and therefore the results are not conclusive at this dose (FIG. 11B, p<0.05).

Effect of NS2 on NFκB Levels.

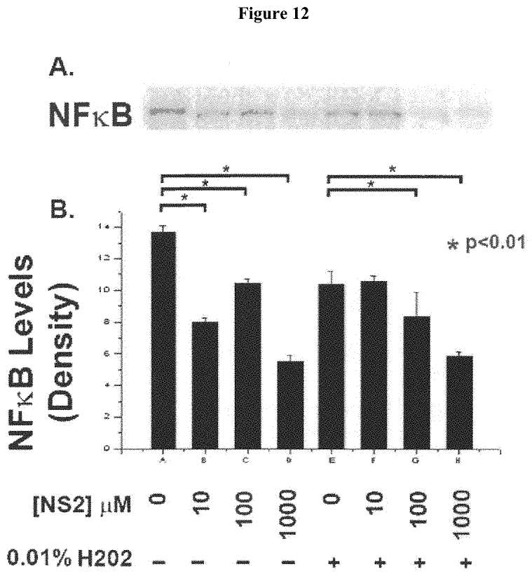

To determine if the loss of NFκB to the nuclei was due to loss of protein overall, the levels of NFκB were examined by Western Blot (FIG. 12A) Western blot analysis of whole cell lysate, which primarily detects cytoplasmic protein levels, showed that NS2 significantly decreased NFκB in non-stimulated cells (FIG. 12B). In the H₂O₂-stimulated cells, only 100 μM or 1 mM NS2 treatments showed significant decreases in NFκB, although as with other analyses here, the 1 mM dose is likely to not give reliable data (FIG. 12B). These data suggest that at least some of the loss of translocation of NFκB is due to loss of protein in the unstimulated cells. The samples analyzed in the Western Blot analysis of FIG. 12A are as follows: Lane 1—Vehicle control; Lane 2—unstimulated treated with 10 μM NS2; Lane 3—unstimulated treated with 100 μM NS2; Lane 4—unstimulated treated with 1 mM NS2; Lane 5—H₂O₂ stimulated Vehicle control; Lane 6—H₂O₂ stimulated treated with 10 μM NS2; Lane 7—H₂O₂ stimulated treated with 100 μM NS2; and Lane 8—H₂O₂ stimulated treated with 1 mM NS2.

Interleukin 1-β Expression is Inhibited by NS2 Treatment of Cardiac Fibroblasts.

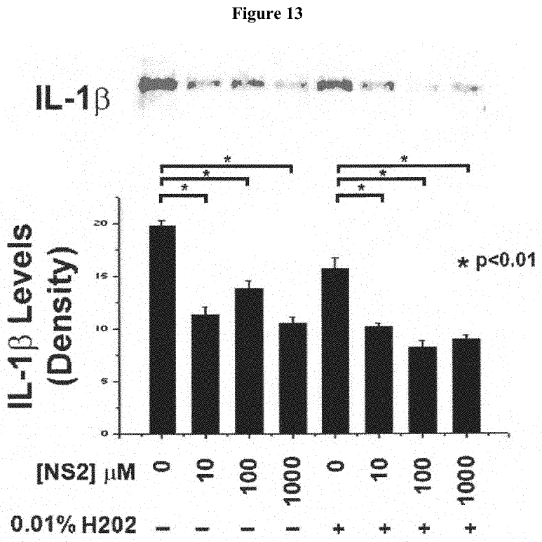

Translocation of NFκB to the nucleus leads to upregulation of a number of pro-inflammatory cytokines, including Interleukin-1β (IL-1β), which can stimulate fibroblasts to transform into myofibroblasts (Baum et al, 2012, Front. Physiol. 3:272 (e journal). To determine if the blockade of NFκB translocation by NS2 had a functional impact on this pathway, the effect of NS2 treatment on IL-1β levels was examined in both unstimulated and H₂O₂ stimulated cardiac fibroblasts. It was found that both unstimulated and H₂O₂ stimulated cells showed high expression of IL-1β by 24 hours after plating (FIG. 13). Unstimulated and H₂O₂-stimulated cells showed significant decreases in IL-1β levels following NS2 treatment (FIG. 13B) (p<0.01). These data suggest that NS2 alters the inflammatory pathway by blocking NFκB translocation and subsequent upregulation of IL-1β. The shut-down of this pro-inflammatory pathway likely plays a role in the inhibition of activation of fibroblasts myofibroblast phenotype. The samples analyzed in the Western Blot analysis of FIG. 13A are as follows: Lane 1—Vehicle control; Lane 2—unstimulated treated with 10 uM NS2; Lane 3—unstimulated treated with 100 uM NS2; Lane 4—unstimulated treated with 1 mM NS2; Lane 5—H₂O₂ stimulated Vehicle control; Lane 6—H₂O₂ stimulated treated with 10 uM NS2; Lane 7—H₂O₂ stimulated treated with 100 uM NS2; and Lane 8—H₂O₂ stimulated treated with 1 mM NS2.

Effect of NS2 on Activation of the MAPK Signaling Pathways in Cardiac Fibroblasts.

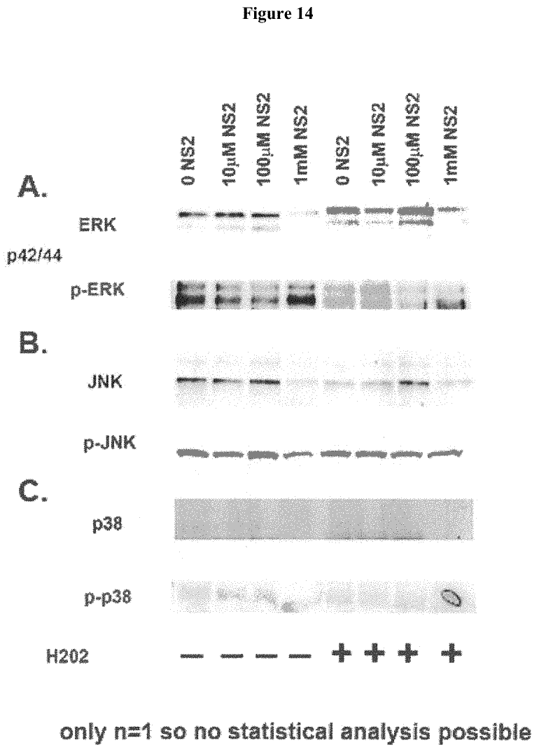

Activation of MAPK pathways has previously been implicated in myofibroblast activation (Dolmatova et al, 2012, Am. J. Physiol Heart Circ Physiol 303(10):H1208-1218.). To determine if these pathways were involved in these specific cells, i.e., the cardiac fibroblasts, the levels and phosphorylation status of ERK, JNK and p38 were examined (FIG. 14). Because only one Western Blot was successful, a reliable analysis was not possible. The antibody for p38 worked very poorly, and thus no information could be ascertained from the Western Blot for p38. The Western Blot for JNK-pJNK showed no changes with NS2 treatment at any level. The ERK/pERK did show changes in the level of ERK phosphorylation although with only a single Western Blot, no clear conclusions could be made. However, because phosphatase inhibitors, which preserve the phosphorylation state of the enzymes during cell lysis, were not present in the cell lysis buffers, no conclusions about changes in phosphorylation state of MAP kinase isoforms can be drawn. The samples analyzed in the Western Blot analysis of FIG. 14A-C are as follows: Lane 1—Vehicle control; Lane 2—unstimulated treated with 10 uM NS2; Lane 3—unstimulated treated with 100 uM NS2; Lane 4—unstimulated treated with 1 mM NS2; Lane 5—H₂O₂ stimulated Vehicle control; Lane 6—H₂O₂ stimulated treated with 10 uM NS2; Lane 7—H₂O₂ stimulated treated with 100 uM NS2; Lane 8—H₂O₂ stimulated treated with 1 mM NS2.

### Discussion:

Studies using small molecule aldehyde trap, NS2, has shown that scavenging of the aldehydes by the trap leads to a decrease in the activation of pro-inflammatory cytokines. In an LPS model of inflammation in the mouse, treatment with NS2 significantly decreased activation of IL-1β, IL-17 and TGF-β, with significantly increasing levels of the anti-inflammatory cytokine Interleukin 10 (IL-10). More importantly, in an oral mucositis model of inflammation caused by radiation of the hamster cheek pouch, treatment with NS2 led to a significant increase in the rate of recovery from injury and a decrease in overall fibrosis at the site of injury over time. Based on previous studies which showed that NS2 limited the activation of IL-1β in a murine LPS model, the prediction has been that NS2 works via limiting the translocation of NFκB to the nucleus of cells. It is shown herein that this mechanism is at play in NS2's ability to limit activation of fibroblasts to the myofibroblast phenotype. This activation model may be ideal for further testing of NS2 analog activity.

Cultured fibroblasts exhibit auto-transformation to the activated myofibroblast phenotype. This transformation is thought to be due to the interaction of the focal adhesion sites to the plastic of the cell culture dishes or cover slips which they are traditionally plated on. The cells “see” the contact with plastic as an injury and upregulate injury pathways such as inflammatory pathways and the MAPK signaling pathway. This leads to changes in cell shape, increases in motility, increased presence of focal adhesions and the presence of α-SMA. α-SMA is a marker for activated myofibroblast phenotype. In fact, it is considered the “gold-standard” marker for fibroblast activation. Western blot analysis of α-SMA protein levels in cells after treatment with 10 μM NS2 showed a significant decrease in the α-SMA protein levels. Overall cell protein levels were not reduced after treatment with 10 μM NS2. The Western Blot data are more indicative of what is occurring over a larger sample size. Overall these data show clearly that NS2 inhibits α-SMA, indicating that it has a role in blocking activation of fibroblasts into myofibroblasts.

Examination of the effects of NS2 on inflammasome activation showed that NS2 significantly decreases NFκB translocation to the nucleus of affected cells, an early event of the pro-inflammatory cytokine IL-1β, which has previously been shown to cause fibroblast activation. This loss of translocation led to a significant downregulation of the pro-inflammatory cytokine IL-1b which has previously been shown to cause fibroblast activation. Taken together it appears that the ability of NS2 to limit fibroblast activation is by blocking the inflammasome activation at the level of NFκB. Analysis of the phosphorylation state of MAPK isoforms was hampered by technical difficulties. However, future studies should investigate the phosphorylation of MAPK isoforms at much earlier time points as well, as these enzymes are often activated early in the fibrotic process.

Stimulation with Hydrogen Peroxide.

In most of the studies done here, cells were tested under two conditions. The first was non-stimulated cells, which cells will auto-activate over time in culture. Addition of H₂O₂ causes more rapid and increased activation of cells. In the studies with H₂O₂ stimulation of the cardiac fibroblasts, treatment with NS2 did not consistently limit changes in α-SMA and NFκB levels. This is likely due to the ongoing presence of H₂O₂ in the media, which gives a continual activation. Non-stimulated cells activate more slowly and to a lesser degree, giving time for the NS2 to block translocation and subsequent activation of the inflammasome. In future studies using fibroblasts, more consistent and physiologically relevant data are likely to be achieved by using unstimulated cells rather than by over stimulating the pathways by exposing the cells to H₂O₂.

Fibroblast Activation as a Model for Studying Drug Analogs.

Cultured fibroblasts are easy to obtain, easy to culture and easy to treat. Cells can be obtained by the method described herein, which gives relatively fewer cells to work with, or by extracting them from neonatal “red tissues” taken together (heart, lung, liver). Additionally, fibroblasts can be purchased from ATCC, a central source for in vitro cells (www.atcc.org). The ATCC can be a source of fibroblasts from epidermis, bladder, uterus and other sources, both murine and human. While the initial tests with NS2 would need to be repeated to confirm activity in the new cell type, based on studies in the literature on α-SMA in a number of fibroblasts of various origins, there is little doubt that it would work in a similar manner as seen in this study. Determining activation is easy in these cells making it simple to determine if NS2 or any analog of NS2 is working. A simple colormetric assay could be developed based on cultured fibroblasts and antibodies directed against α-SMA. This assay could be miniaturized and automated, making this a simple and inexpensive model for testing activity of any compound which is thought to limit fibroblast activation. Confirming the results of the automated assay is also simple, requiring only a few Western Blots and/or microscopic analysis for NFκB translocation. Additionally, nuclear extract studies could also be done to determine if a compound limits NFκB translocation.

### Conclusion:

These forgoing studies show that NS2 limits activation of fibroblasts to the myofibroblast phenotype by blocking NFκB translocation to the nucleus, thus limiting activation of the pro-inflammatory pathways and subsequent fibrosis. Additionally, these studies give a simple model for identification of other compounds which may have activity similar to NS2. Further studies on animal models can be used to confirm if NS2 can limit fibroblast activation in vivo and limit injury based fibrosis.

### Example 15: Assay Results for Aldehyde Adduct Formation, 4HNE Consumption, and Equilibration Over Time

Five compounds were examined:
2-(3-aminoquinolin-2-yl)propan-2-ol
2-(3-amino-5-chloroquinolin-2yl)propan-2-ol
2-(3-amino-7-chloroquinolin-2-yl)propan-2-ol
2-(3-amino-8-chloroquinolin-2-yl)propan-2-ol
2-(3-amino-6-bromoquinolin-2-yl)propan-2-ol

NS2 was also examined for comparison.

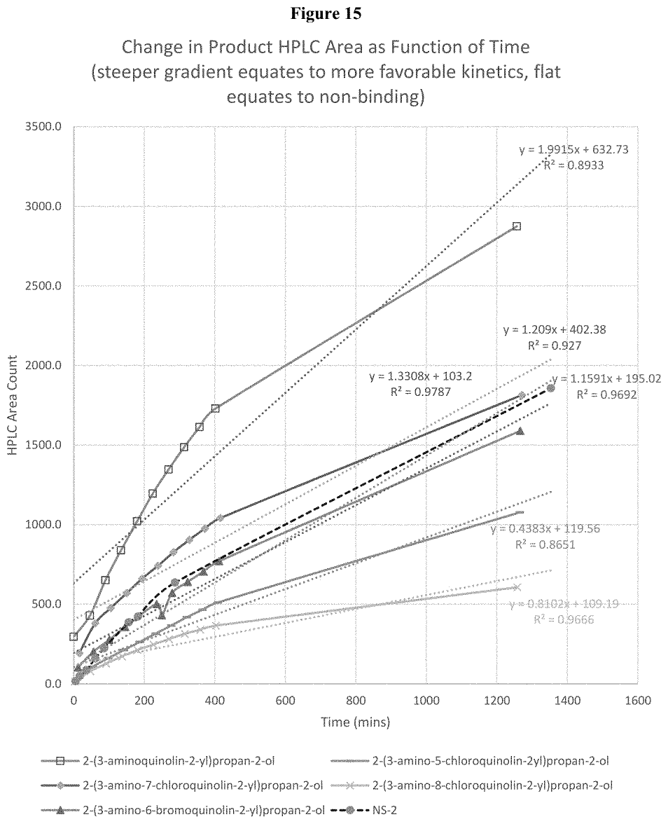

FIG. 15 shows rates of formation of aldehyde adducts over a 23 h time period for NS2 and the exemplary compounds. It was found that all samples bind (+ve increase in product HPLC peak over time), although one binds less well than the others. It is not possible to conclude if this is the result of poor dissociation (from cyclodextrin) or poor interaction with the aldehyde. Best fit lines over this period give excellent fit to data. Rate of product peak increase can be used as an approximation of binding kinetics; however, it does not provide any way to separate kinetics of dissociation (from cyclodextrin) and kinetics of binding. It can be used to relatively rank each of the samples examined, including NS2. The data were first evaluated over a 7 h time window. This resulted in the following rankings from most effective to least:

| | |
|---|---|
| 1. 2-(3-aminoquinolin-2-yl)propan-2-ol | (Gradient 3.68, |
| | R. Sq. 0.993) |
| 2. NS-2 | (Gradient 2.22, |
| | R. Sq. 0.996) |
| 3. 2-(3-amino-7-chloroquinolin-2-yl)propan-2-ol | (Gradient 2.02, |
| | R. Sq. 0.984) |
| 4. 2-(3-amino-6-bromoquinolin-2-yl)propan-2-ol | (Gradient 1.63, |
| | R. Sq. 0.983) |
| 5. 2-(3-amino-8-chloroquinolin-2-yl)propan-2-ol | (Gradient 1.18, |
| | R. Sq. 0.997) |
| 6. 2-(3-amino-5-chloroquinolin-2yl)propan-2-ol | (Gradient 0.86, |
| | R. Sq. 0.983) |

Similar results were obtained when the window was extended to 23 h. However, two of the compounds yielded lower R. Sq. values in this context.

| | |
|---|---|
| 1. 2-(3-aminoquinolin-2-yl)propan-2-ol | (Gradient 1.99, |
| | R. Sq. 0.893) |
| 2. NS-2 | (Gradient 1.33, |
| | R. Sq. 0.979) |
| 3. 2-(3-amino-7-chloroquinolin-2-yl)propan-2-ol | (Gradient 1.21, |
| | R. Sq. 0.927) |
| 4. 2-(3-amino-6-bromoquinolin-2-yl)propan-2-ol | (Gradient 1.16, |
| | R. Sq. 0.969) |
| 5. 2-(3-amino-8-chloroquinolin-2-yl)propan-2-ol | (Gradient 0.81, |
| | R. Sq. 0.967) |
| 6. 2-(3-amino-5-chloroquinolin-2yl)propan-2-ol | (Gradient 0.44, |
| | R. Sq. 0.967) |

One possible explanation is that the two kinetic components (dissociation and binding) are no longer balanced and one is the determining factor. A follow-up experiment would be to closely track one sample over 60-70 injections to establish where the slope change occurs (this would potentially give access point to separate dissociation and binding kinetic components).

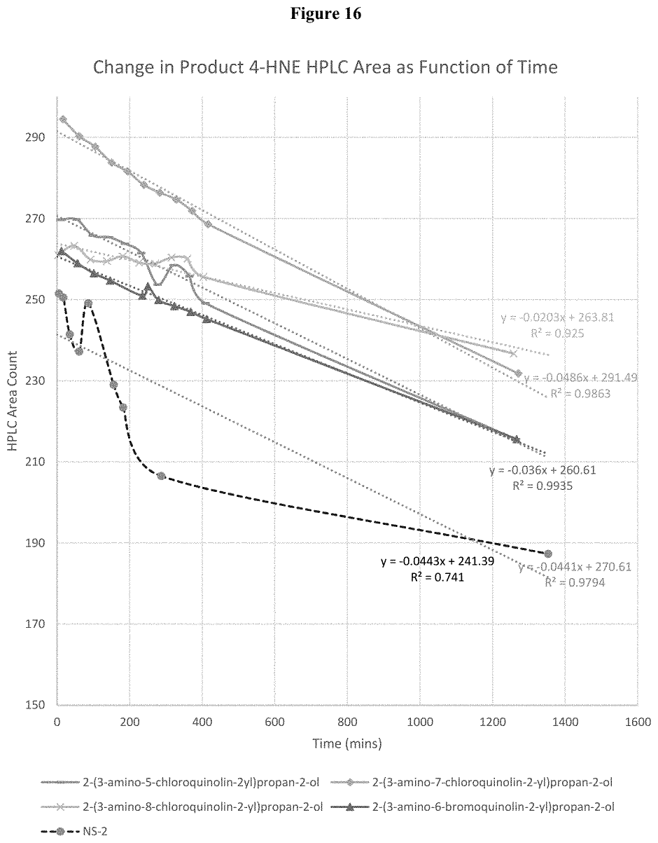

FIG. 16 shows consumption of 4HNE over time (23-hour formation period) for NS2 and other exemplary compounds. 5 of 6 samples show consumption of 4HNE. One sample (2-(3-aminoquinolin-2-yl)propan-2-ol) overlaps 4HNE HPLC peak using current method. Best fit lines over this period give poorer fit to data than product formation data. Rate of 4HNE consumption can be used as an approximation of binding kinetics. As before, the data do not provide any way to separate kinetics of dissociation (from cyclodextrin) and kinetics of binding. The data were used to rank relatively each of the samples examined, including NS-2 but excluding 2-(3-aminoquinolin-2-yl)propan-2-ol. During the first 7 h, the data yielded the following rankings from most effective to least (analysis at 254 nm):

| | |
|---|---|
| 1. NS-2 | (Gradient −0.15, |
| | R. Sq. 0.903) |
| 2. 2-(3-amino-7-chloroquinolin-2-yl)propan-2-ol | (Gradient −0.06, |
| | R. Sq. 0.991) |
| 3. 2-(3-amino-5-chloroquinolin-2yl)propan-2-ol | (Gradient −0.05, |
| | R. Sq. 0.898) |
| 4. 2-(3-amino-6-bromoquinolin-2-yl)propan-2-ol | (Gradient −0.04, |
| | R. Sq. 0.971) |
| 5. 2-(3-amino-8-chloroquinolin-2-yl)propan-2-ol | (Gradient −0.01, |
| | R. Sq. 0.461) |

Analysis at 23 h provided the following
rankings from most effective to least:

| | |
|---|---|
| 1. 2-(3-amino-7-chloroquinolin-2-yl)propan-2-ol | (Gradient −0.05, |
| | R. Sq. 0.986) |
| 2. 2-(3-amino-5-chloroquinolin-2yl)propan-2-ol | (Gradient −0.04, |
| | R. Sq. 0.979) |
| 3. NS-2 | (Gradient −0.04, |
| | R. Sq. 0.741) |
| 4. 2-(3-amino-6-bromoquinolin-2-yl)propan-2-ol | (Gradient −0.04, |
| | R. Sq. 0.994) |
| 5. 2-(3-amino-8-chloroquinolin-2-yl)propan-2-ol | (Gradient −0.02, |
| | R. Sq. 0.925) |

Note, differences between bold numbers are very small (Gradient numbers rounded to value shown).

The following table summarizes the above data:

TABLE 2

| | Formation | | Consumption | |
|---|---|---|---|---|
| | of Product | | of 4HNE | |
| | 7 | 23 | 7 | 23 |
| Compound | Hours | Hours | Hours | Hours^{¶} |
| 2-(3-aminoquinolin-2-yl)propan- | 1 | 1 | n/a | n/a |
| 2-ol | | | | |
| NS-2 | 2 | 2 | 1 | 3 |
| 2-(3-amino-7-chloroquinolin-2- | 3 | 3 | 2 | 1 |
| yl)propan-2-ol | | | | |
| 2-(3-amino-6-bromoquinolin-2- | 4 | 4 | 4 | 4 |
| yl)propan-2-ol | | | | |
| 2-(3-amino-8-chloroquinolin-2- | 5 | 5 | 5 | 5 |
| yl)propan-2-ol | | | | |
| 2-(3-amino-5-chloroquinolin- | 6 | 6 | 3 | 2 |
| 2yl)propan-2-ol | | | | |

^{¶}Small differences between samples ranking 1-4, essentially identical

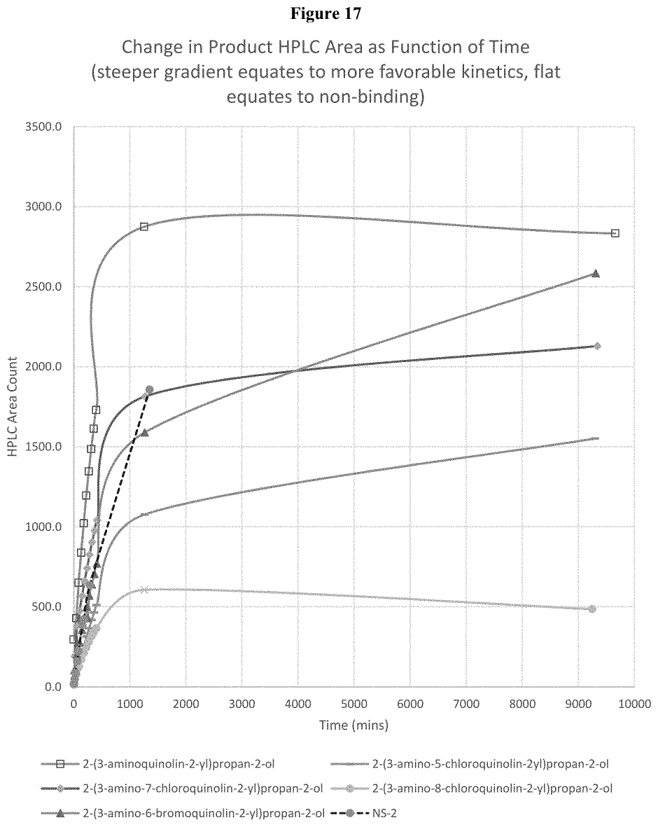

FIG. 17 shows rates of formation of aldehyde adducts over a 1 week time period for NS2 and exemplary compounds of the present invention to measure whether compounds reached equilibrium. During this time period 3 of the 5 samples reached equilibrium.

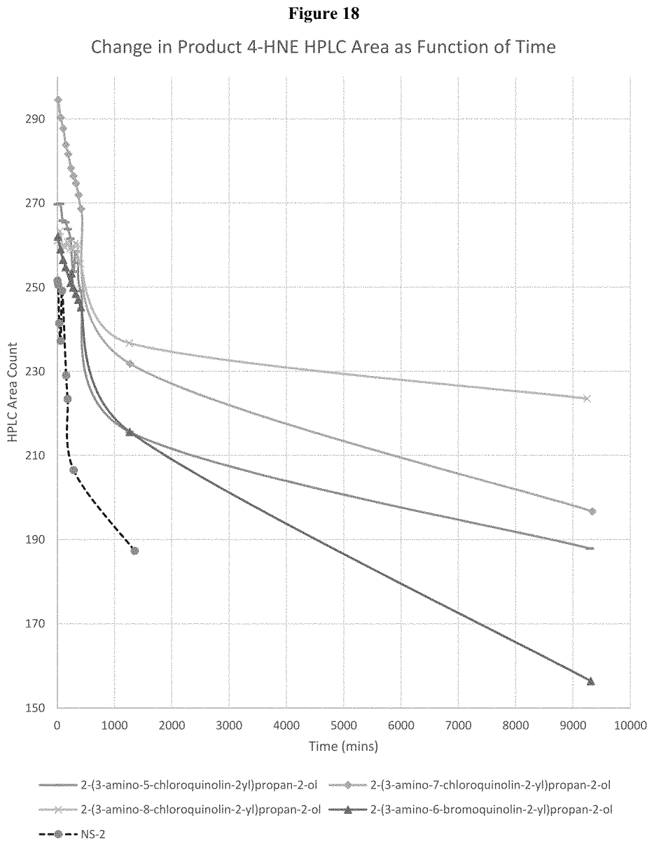

FIG. 18 shows consumption of 4HNE over a 1 week time period for NS2 and exemplary compounds of the present invention to measure whether compounds reached equilibrium during this time period. The samples appeared to reach equilibrium, with the ongoing decrease in HNE amounts possibly due to another degradative pathway. This is because the decrease in HNE is greater than the corresponding increase in adduct (shown in FIG. 17) for at least 2-(3-amino-8 chloroquinolin-2-yl)propan-2-ol and 2-(3-amino-7-chloroquinolin-2-yl)propan-2-ol.

All publications, patents, patent applications and other documents cited in this application are hereby incorporated by reference in their entireties for all purposes to the same extent as if each individual publication, patent, patent application or other document were individually indicated to be incorporated by reference for all purposes.

We claim:

1. A method of preparing a conjugate of formula I: 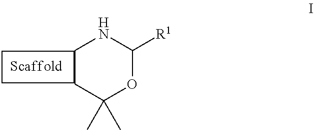
or a pharmaceutically acceptable salt thereof, wherein:
Scaffold is 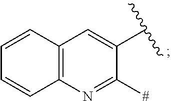 [symbol]
is the point of attachment to the amino group;
# is the point of attachment to the carbinol group; and
R¹ is the side-chain of a biologically relevant aldehyde;
comprising the steps of:
(a) providing a compound of formula A: 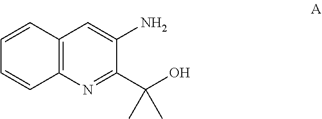
or a pharmaceutically acceptable salt thereof; and
(b) contacting the compound of formula A with the biologically relevant aldehyde to form the conjugate of formula I;
wherein the biologically relevant aldehyde is succinic semi-aldehyde.

2-17. (canceled)

18. A conjugate of formula I: 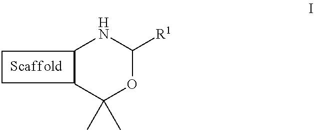
wherein Scaffold is: 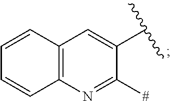 [symbol]
is the point of attachment to the amino group;
# is the point of attachment to the carbinol group; and
R¹ is the side-chain of succinic semi-aldehyde.

19-48. (canceled)

49. A method of treating succinic semi-aldehyde dehydrogenase deficiency (SSADHD) in a subject in need thereof, comprising the step of administering to the subject a compound of formula A: 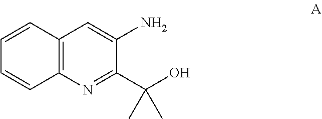
or a pharmaceutically acceptable salt thereof;
wherein the compound of formula A reacts with succinic semi-aldehyde to form a conjugate of formula I: 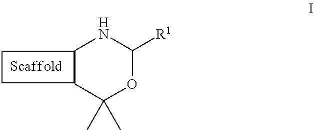
wherein Scaffold is: 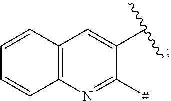 [symbol]
is the point of attachment to the amino group;
# is the point of attachment to the carbinol group; and
R¹ is the side-chain of succinic semi-aldehyde.

50. The method of claim 49, wherein the method treats developmental delay, hypotonia, severe expressive language impairment, obsessive-compulsive disorder, epilepsy, ADHD, or aggression associated with SSADHD.

51. The method of claim 49, wherein the method reduces accumulation of GABA in the subject caused by SSADHD.

52. The method of claim 49, wherein the method reduces accumulation of GHB in the subject caused by SSADHD.

53. The method of claim 49, wherein the method reduces accumulation of GABA and GHB in the subject caused by SSADHD.

54. The method of claim 49, wherein the subject is a human.

55. A method of reducing accumulation of GABA and/or GHB ex vivo, comprising contacting succinic semi-aldehyde in a biological sample with a compound of formula A: 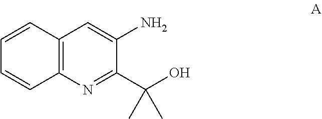
or a pharmaceutically acceptable salt thereof.

56. The method of claim 55, wherein the method comprises contacting brain slices of B6.129-Aldh5a1^{tm1Kmg/}J (SSADH null) mice with the compound of formula A or a pharmaceutically acceptable salt thereof.
